(19) United States
(12) Patent Application Publication
(10) Pub. No.: US 2024/0402880 A1
(43) Pub. Date: Dec. 5, 2024

(54) METHODS AND USER INTERFACES FOR MANAGING AND ACCESSING WORKOUT CONTENT

(71) Applicant: Apple Inc., Cupertino, CA (US)

(72) Inventors: Brett L. LAREAU, San Jose, CA (US); Julie A. ARNEY, Los Gatos, CA (US); Chandler S. BACHMAN, Cupertino, CA (US); Margaret A. BIGNELL, San Francisco, CA (US); Jay K. BLAHNIK, Venice, CA (US); Gary I. BUTCHER, Los Gatos, CA (US); Andrew K. PARR, San Jose, CA (US); Matthew M. SUN, Cupertino, CA (US); Eduardo VALENCIA PAZ, San Francisco, CA (US); Lessa WAKUTSU, San Francisco, CA (US)

(21) Appl. No.: 18/608,832

(22) Filed: Mar. 18, 2024

Related U.S. Application Data
(60) Provisional application No. 63/470,962, filed on Jun. 4, 2023.

(51) Int. Cl. G06F 3/0482; A63B 24/00; A63B 71/06

(52) U.S. Cl. CPC G06F 3/0482; A63B 24/0075; A63B 71/0622; A63B 2024/0078; A63B 2071/0625

(57) ABSTRACT

The present disclosure generally relates to providing content including fitness-related content.

### CROSS-REFERENCE TO RELATED APPLICATIONS

This application claims priority to U.S. Provisional Application No. 63/470,962, filed Jun. 4, 2023 and entitled “METHODS AND USER INTERFACES FOR MANAGING AND ACCESSING WORKOUT CONTENT,” the entire disclosure of which is hereby incorporated by reference.

### FIELD

The present disclosure relates generally to computer user interfaces, and more specifically to techniques for providing and accessing workout content.

### BACKGROUND

Electronic devices can be used for accessing workout content (e.g., multimedia workout content), including content that is local storied or delivered via a service.

### BRIEF SUMMARY

Some techniques for providing and accessing workout content using electronic devices, however, are generally cumbersome and inefficient. For example, some existing techniques use a complex and time-consuming user interface, which may include multiple key presses or keystrokes. Existing techniques require more time than necessary, wasting user time and device energy. This latter consideration is particularly important in battery-operated devices.

Accordingly, the present technique provides electronic devices with faster, more efficient methods and interfaces for providing and accessing workout content. Such methods and interfaces optionally complement or replace other methods for providing and accessing workout content. Such methods and interfaces reduce the cognitive burden on a user and produce a more efficient human-machine interface. For battery-operated computing devices, such methods and interfaces conserve power and increase the time between battery charges.

In accordance with some embodiments, a method performed at a computer system that is in communication with one or more display generation components and one or more input devices, is described. The method comprising: receiving, via the one or more input devices, a first set of user inputs, including: one or more user inputs corresponding to user selection of one or more workout days; and one or more user inputs corresponding to user selection of one or more workout types; in response to receiving the first set of user inputs, displaying, via the one or more display generation components, a workout plan user interface, including: a representation of a first day that is selected based on the one or more user inputs corresponding to user selection of one or more workout days; a representation of a second day different from the first day that is selected based on the one or more user inputs corresponding to user selection of one or more workout days; an indication of a first workout corresponding to the first day and that is selected based on the one or more user inputs corresponding to user selection of one or more workout types; and an indication of a second workout corresponding to the second day and that is selected based on the one or more user inputs corresponding to user selection of one or more workout types.

In accordance with some embodiments, a non-transitory computer-readable storage medium storing one or more programs configured to be executed by one or more processors of a computer system that is in communication with one or more display generation components and one or more input devices, is described. The one or more programs including instructions for: receiving, via the one or more input devices, a first set of user inputs, including: one or more user inputs corresponding to user selection of one or more workout days; and one or more user inputs corresponding to user selection of one or more workout types; in response to receiving the first set of user inputs, displaying, via the one or more display generation components, a workout plan user interface, including: a representation of a first day that is selected based on the one or more user inputs corresponding to user selection of one or more workout days; a representation of a second day different from the first day that is selected based on the one or more user inputs corresponding to user selection of one or more workout days; an indication of a first workout corresponding to the first day and that is selected based on the one or more user inputs corresponding to user selection of one or more workout types; and an indication of a second workout corresponding to the second day and that is selected based on the one or more user inputs corresponding to user selection of one or more workout types.

In accordance with some embodiments, a transitory computer-readable storage medium storing one or more programs configured to be executed by one or more processors of a computer system that is in communication with one or more display generation components and one or more input devices, is described. The one or more programs including instructions for: receiving, via the one or more input devices, a first set of user inputs, including: one or more user inputs corresponding to user selection of one or more workout days; and one or more user inputs corresponding to user selection of one or more workout types; in response to receiving the first set of user inputs, displaying, via the one or more display generation components, a workout plan user interface, including: a representation of a first day that is selected based on the one or more user inputs corresponding to user selection of one or more workout days; a representation of a second day different from the first day that is selected based on the one or more user inputs corresponding to user selection of one or more workout days; an indication of a first workout corresponding to the first day and that is selected based on the one or more user inputs corresponding to user selection of one or more workout types; and an indication of a second workout corresponding to the second day and that is selected based on the one or more user inputs corresponding to user selection of one or more workout types.

In accordance with some embodiments, a computer system configured to communicate with one or more display generation components and one or more input devices, is described. The computer system comprising: one or more processors; and memory storing one or more programs configured to be executed by the one or more processors. The one or more programs including instructions for: receiving, via the one or more input devices, a first set of user inputs, including: one or more user inputs corresponding to user selection of one or more workout days; and one or more user inputs corresponding to user selection of one or more workout types; in response to receiving the first set of user inputs, displaying, via the one or more display generation components, a workout plan user interface, including: a representation of a first day that is selected based on the one or more user inputs corresponding to user selection of one or more workout days; a representation of a second day different from the first day that is selected based on the one or more user inputs corresponding to user selection of one or more workout days; an indication of a first workout corresponding to the first day and that is selected based on the one or more user inputs corresponding to user selection of one or more workout types; and an indication of a second workout corresponding to the second day and that is selected based on the one or more user inputs corresponding to user selection of one or more workout types.

In accordance with some embodiments, a computer system configured to communicate with one or more display generation components and one or more input devices, is described. The computer system comprising: means for receiving, via the one or more input devices, a first set of user inputs, including: one or more user inputs corresponding to user selection of one or more workout days; and one or more user inputs corresponding to user selection of one or more workout types; means, in response to receiving the first set of user inputs, for displaying, via the one or more display generation components, a workout plan user interface, including: a representation of a first day that is selected based on the one or more user inputs corresponding to user selection of one or more workout days; a representation of a second day different from the first day that is selected based on the one or more user inputs corresponding to user selection of one or more workout days; an indication of a first workout corresponding to the first day and that is selected based on the one or more user inputs corresponding to user selection of one or more workout types; and an indication of a second workout corresponding to the second day and that is selected based on the one or more user inputs corresponding to user selection of one or more workout types.

In accordance with some embodiments, a computer program product, comprising one or more programs configured to be executed by one or more processors of a computer system that is in communication with one or more display generation components and one or more input devices, is described. The one or more programs including instructions for: receiving, via the one or more input devices, a first set of user inputs, including: one or more user inputs corresponding to user selection of one or more workout days; and one or more user inputs corresponding to user selection of one or more workout types; in response to receiving the first set of user inputs, displaying, via the one or more display generation components, a workout plan user interface, including: a representation of a first day that is selected based on the one or more user inputs corresponding to user selection of one or more workout days; a representation of a second day different from the first day that is selected based on the one or more user inputs corresponding to user selection of one or more workout days; an indication of a first workout corresponding to the first day and that is selected based on the one or more user inputs corresponding to user selection of one or more workout types; and an indication of a second workout corresponding to the second day and that is selected based on the one or more user inputs corresponding to user selection of one or more workout types.

In accordance with some embodiments, a method performed at a computer system that is in communication with one or more display generation components and one or more input devices, is described. The method comprising: receiving, via the one or more input devices, a first set of user inputs; and in response to receiving the first set of user inputs, displaying, via the one or more display generation components, a workout selection user interface, including: displaying, in a first region of the workout selection user interface, a first workout platter associated with a first respective workout; and displaying, in a second region of the workout selection user interface different from the first region, a second workout platter different from the first workout platter and associated with a second respective workout different from the first respective workout, wherein: in accordance with a determination that a first set of criteria is satisfied, including a first criterion that is satisfied when a user-defined workout plan is active, the first workout platter corresponds to a first workout that is selected based on the user-defined workout plan; and in accordance with a determination that a user-defined workout plan is not active, the first workout platter corresponds to a second workout different from the first workout.

In accordance with some embodiments, a non-transitory computer-readable storage medium storing one or more programs configured to be executed by one or more processors of a computer system that is in communication with one or more display generation components and one or more input devices, is described. The one or more programs including instructions for: receiving, via the one or more input devices, a first set of user inputs; and in response to receiving the first set of user inputs, displaying, via the one or more display generation components, a workout selection user interface, including: displaying, in a first region of the workout selection user interface, a first workout platter associated with a first respective workout; and displaying, in a second region of the workout selection user interface different from the first region, a second workout platter different from the first workout platter and associated with a second respective workout different from the first respective workout, wherein: in accordance with a determination that a first set of criteria is satisfied, including a first criterion that is satisfied when a user-defined workout plan is active, the first workout platter corresponds to a first workout that is selected based on the user-defined workout plan; and in accordance with a determination that a user-defined workout plan is not active, the first workout platter corresponds to a second workout different from the first workout.

In accordance with some embodiments, a transitory computer-readable storage medium storing one or more programs configured to be executed by one or more processors of a computer system that is in communication with one or more display generation components and one or more input devices, is described. The one or more programs including instructions for: receiving, via the one or more input devices, a first set of user inputs; and in response to receiving the first set of user inputs, displaying, via the one or more display generation components, a workout selection user interface, including: displaying, in a first region of the workout selection user interface, a first workout platter associated with a first respective workout; and displaying, in a second region of the workout selection user interface different from the first region, a second workout platter different from the first workout platter and associated with a second respective workout different from the first respective workout, wherein: in accordance with a determination that a first set of criteria is satisfied, including a first criterion that is satisfied when a user-defined workout plan is active, the first workout platter corresponds to a first workout that is selected based on the user-defined workout plan; and in accordance with a determination that a user-defined workout plan is not active, the first workout platter corresponds to a second workout different from the first workout.

In accordance with some embodiments, a computer system configured to communicate with one or more display generation components and one or more input devices, is described. The computer system comprising: one or more processors; and memory storing one or more programs configured to be executed by the one or more processors. The one or more programs including instructions for: receiving, via the one or more input devices, a first set of user inputs; and in response to receiving the first set of user inputs, displaying, via the one or more display generation components, a workout selection user interface, including: displaying, in a first region of the workout selection user interface, a first workout platter associated with a first respective workout; and displaying, in a second region of the workout selection user interface different from the first region, a second workout platter different from the first workout platter and associated with a second respective workout different from the first respective workout, wherein: in accordance with a determination that a first set of criteria is satisfied, including a first criterion that is satisfied when a user-defined workout plan is active, the first workout platter corresponds to a first workout that is selected based on the user-defined workout plan; and in accordance with a determination that a user-defined workout plan is not active, the first workout platter corresponds to a second workout different from the first workout.

In accordance with some embodiments, a computer system configured to communicate with one or more display generation components and one or more input devices, is described. The computer system comprising: means for receiving, via the one or more input devices, a first set of user inputs; and means, in response to receiving the first set of user inputs, for displaying, via the one or more display generation components, a workout selection user interface, including: displaying, in a first region of the workout selection user interface, a first workout platter associated with a first respective workout; and displaying, in a second region of the workout selection user interface different from the first region, a second workout platter different from the first workout platter and associated with a second respective workout different from the first respective workout, wherein: in accordance with a determination that a first set of criteria is satisfied, including a first criterion that is satisfied when a user-defined workout plan is active, the first workout platter corresponds to a first workout that is selected based on the user-defined workout plan; and in accordance with a determination that a user-defined workout plan is not active, the first workout platter corresponds to a second workout different from the first workout.

In accordance with some embodiments, a computer program product, comprising one or more programs configured to be executed by one or more processors of a computer system that is in communication with one or more display generation components and one or more input devices, is described. The one or more programs including instructions for: receiving, via the one or more input devices, a first set of user inputs; and in response to receiving the first set of user inputs, displaying, via the one or more display generation components, a workout selection user interface, including: displaying, in a first region of the workout selection user interface, a first workout platter associated with a first respective workout; and displaying, in a second region of the workout selection user interface different from the first region, a second workout platter different from the first workout platter and associated with a second respective workout different from the first respective workout, wherein: in accordance with a determination that a first set of criteria is satisfied, including a first criterion that is satisfied when a user-defined workout plan is active, the first workout platter corresponds to a first workout that is selected based on the user-defined workout plan; and in accordance with a determination that a user-defined workout plan is not active, the first workout platter corresponds to a second workout different from the first workout.

In accordance with some embodiments, a method performed at a computer system that is in communication with one or more display generation components and one or more input devices: receiving, via the one or more input devices, a first set of user inputs; and in response to receiving the first set of user inputs, displaying, via the one or more display generation components, a workout selection user interface, including: displaying, in a first region of the workout selection user interface, a first workout platter associated with a first respective workout; and displaying, in a second region of the workout selection user interface different from the first region, a second workout platter different from the first workout platter and associated with a second respective workout different from the first respective workout, wherein: in accordance with a determination that a first set of criteria is satisfied, including a first criterion that is satisfied when a user-defined workout plan is active for a user of the computer system wherein the user-defined workout plan is generated based on one or more user inputs received at a first external device separate from the computer system, the first workout platter corresponds to a first workout that is selected based on the user-defined workout plan; and in accordance with a determination that a user-defined workout plan is not active, the first workout platter corresponds to a second workout different from the first workout.

In accordance with some embodiments, a non-transitory computer-readable storage medium storing one or more programs configured to be executed by one or more processors of a computer system that is in communication with one or more display generation components and one or more input devices, is described. The one or more programs including instructions for: receiving, via the one or more input devices, a first set of user inputs; and in response to receiving the first set of user inputs, displaying, via the one or more display generation components, a workout selection user interface, including: displaying, in a first region of the workout selection user interface, a first workout platter associated with a first respective workout; and displaying, in a second region of the workout selection user interface different from the first region, a second workout platter different from the first workout platter and associated with a second respective workout different from the first respective workout, wherein: in accordance with a determination that a first set of criteria is satisfied, including a first criterion that is satisfied when a user-defined workout plan is active for a user of the computer system wherein the user-defined workout plan is generated based on one or more user inputs received at a first external device separate from the computer system, the first workout platter corresponds to a first workout that is selected based on the user-defined workout plan; and in accordance with a determination that a user-defined workout plan is not active, the first workout platter corresponds to a second workout different from the first workout.

In accordance with some embodiments, a transitory computer-readable storage medium storing one or more programs configured to be executed by one or more processors of a computer system that is in communication with one or more display generation components and one or more input devices, is described. The one or more programs including instructions for: receiving, via the one or more input devices, a first set of user inputs; and in response to receiving the first set of user inputs, displaying, via the one or more display generation components, a workout selection user interface, including: displaying, in a first region of the workout selection user interface, a first workout platter associated with a first respective workout; and displaying, in a second region of the workout selection user interface different from the first region, a second workout platter different from the first workout platter and associated with a second respective workout different from the first respective workout, wherein: in accordance with a determination that a first set of criteria is satisfied, including a first criterion that is satisfied when a user-defined workout plan is active for a user of the computer system wherein the user-defined workout plan is generated based on one or more user inputs received at a first external device separate from the computer system, the first workout platter corresponds to a first workout that is selected based on the user-defined workout plan; and in accordance with a determination that a user-defined workout plan is not active, the first workout platter corresponds to a second workout different from the first workout.

In accordance with some embodiments, a computer system configured to communicate with one or more display generation components and one or more input devices, is described. The computer system comprising: one or more processors; and memory storing one or more programs configured to be executed by the one or more processors. The one or more programs including instructions for: receiving, via the one or more input devices, a first set of user inputs; and in response to receiving the first set of user inputs, displaying, via the one or more display generation components, a workout selection user interface, including: displaying, in a first region of the workout selection user interface, a first workout platter associated with a first respective workout; and displaying, in a second region of the workout selection user interface different from the first region, a second workout platter different from the first workout platter and associated with a second respective workout different from the first respective workout, wherein: in accordance with a determination that a first set of criteria is satisfied, including a first criterion that is satisfied when a user-defined workout plan is active for a user of the computer system wherein the user-defined workout plan is generated based on one or more user inputs received at a first external device separate from the computer system, the first workout platter corresponds to a first workout that is selected based on the user-defined workout plan; and in accordance with a determination that a user-defined workout plan is not active, the first workout platter corresponds to a second workout different from the first workout.

In accordance with some embodiments, a computer system configured to communicate with one or more display generation components and one or more input devices, is described. The computer system comprising: means for receiving, via the one or more input devices, a first set of user inputs; and means, in response to receiving the first set of user inputs, for displaying, via the one or more display generation components, a workout selection user interface, including: displaying, in a first region of the workout selection user interface, a first workout platter associated with a first respective workout; and displaying, in a second region of the workout selection user interface different from the first region, a second workout platter different from the first workout platter and associated with a second respective workout different from the first respective workout, wherein: in accordance with a determination that a first set of criteria is satisfied, including a first criterion that is satisfied when a user-defined workout plan is active for a user of the computer system wherein the user-defined workout plan is generated based on one or more user inputs received at a first external device separate from the computer system, the first workout platter corresponds to a first workout that is selected based on the user-defined workout plan; and in accordance with a determination that a user-defined workout plan is not active, the first workout platter corresponds to a second workout different from the first workout.

In accordance with some embodiments, a computer program product, comprising one or more programs configured to be executed by one or more processors of a computer system that is in communication with one or more display generation components and one or more input devices, is described. The one or more programs including instructions for: receiving, via the one or more input devices, a first set of user inputs; and in response to receiving the first set of user inputs, displaying, via the one or more display generation components, a workout selection user interface, including: displaying, in a first region of the workout selection user interface, a first workout platter associated with a first respective workout; and displaying, in a second region of the workout selection user interface different from the first region, a second workout platter different from the first workout platter and associated with a second respective workout different from the first respective workout, wherein: in accordance with a determination that a first set of criteria is satisfied, including a first criterion that is satisfied when a user-defined workout plan is active for a user of the computer system wherein the user-defined workout plan is generated based on one or more user inputs received at a first external device separate from the computer system, the first workout platter corresponds to a first workout that is selected based on the user-defined workout plan; and in accordance with a determination that a user-defined workout plan is not active, the first workout platter corresponds to a second workout different from the first workout.

In accordance with some embodiments, a method performed at a computer system that is in communication with one or more display generation components and one or more input devices, is described. The method comprising: receiving, via the one or more input devices, a first user input corresponding to a request to initiate a workout session corresponding to a first collection of workouts, wherein the first collection of workouts includes a plurality of workouts arranged in an ordered sequence, including a first workout and a second workout different from the first workout; in response to receiving the first user input, displaying, via the one or more display generation components, visual content corresponding to a first respective workout of the plurality of workouts; and subsequent to displaying the visual content corresponding to the first respective workout: in accordance with a determination that the first collection of workouts includes a subsequent workout that follows the first respective workout in the ordered sequence, displaying, via the one or more display generation components, a first selectable object that is selectable to initiate the subsequent workout in the first collection of workouts; and in accordance with a determination that the first respective workout is a final workout in the first collection of workouts, displaying, via the one or more display generation components, a workout summary user interface, including concurrently displaying: a first workout metric that corresponds to the first respective workout but does not correspond to at least some of the other workouts in the first collection of workouts; and a second workout metric that is cumulative for the first collection of workouts.

In accordance with some embodiments, a non-transitory computer-readable storage medium storing one or more programs configured to be executed by one or more processors of a computer system that is in communication with one or more display generation components and one or more input devices, is described. The one or more programs including instructions for: receiving, via the one or more input devices, a first user input corresponding to a request to initiate a workout session corresponding to a first collection of workouts, wherein the first collection of workouts includes a plurality of workouts arranged in an ordered sequence, including a first workout and a second workout different from the first workout; in response to receiving the first user input, displaying, via the one or more display generation components, visual content corresponding to a first respective workout of the plurality of workouts; and subsequent to displaying the visual content corresponding to the first respective workout: in accordance with a determination that the first collection of workouts includes a subsequent workout that follows the first respective workout in the ordered sequence, displaying, via the one or more display generation components, a first selectable object that is selectable to initiate the subsequent workout in the first collection of workouts; and in accordance with a determination that the first respective workout is a final workout in the first collection of workouts, displaying, via the one or more display generation components, a workout summary user interface, including concurrently displaying: a first workout metric that corresponds to the first respective workout but does not correspond to at least some of the other workouts in the first collection of workouts; and a second workout metric that is cumulative for the first collection of workouts.

In accordance with some embodiments, a transitory computer-readable storage medium storing one or more programs configured to be executed by one or more processors of a computer system that is in communication with one or more display generation components and one or more input devices, is described. The one or more programs including instructions for: receiving, via the one or more input devices, a first user input corresponding to a request to initiate a workout session corresponding to a first collection of workouts, wherein the first collection of workouts includes a plurality of workouts arranged in an ordered sequence, including a first workout and a second workout different from the first workout; in response to receiving the first user input, displaying, via the one or more display generation components, visual content corresponding to a first respective workout of the plurality of workouts; and subsequent to displaying the visual content corresponding to the first respective workout: in accordance with a determination that the first collection of workouts includes a subsequent workout that follows the first respective workout in the ordered sequence, displaying, via the one or more display generation components, a first selectable object that is selectable to initiate the subsequent workout in the first collection of workouts; and in accordance with a determination that the first respective workout is a final workout in the first collection of workouts, displaying, via the one or more display generation components, a workout summary user interface, including concurrently displaying: a first workout metric that corresponds to the first respective workout but does not correspond to at least some of the other workouts in the first collection of workouts; and a second workout metric that is cumulative for the first collection of workouts.

In accordance with some embodiments, a computer system configured to communicate with one or more display generation components and one or more input devices, is described. The computer system comprising: one or more processors; and memory storing one or more programs configured to be executed by the one or more processors. The one or more programs including instructions for: receiving, via the one or more input devices, a first user input corresponding to a request to initiate a workout session corresponding to a first collection of workouts, wherein the first collection of workouts includes a plurality of workouts arranged in an ordered sequence, including a first workout and a second workout different from the first workout; in response to receiving the first user input, displaying, via the one or more display generation components, visual content corresponding to a first respective workout of the plurality of workouts; and subsequent to displaying the visual content corresponding to the first respective workout: in accordance with a determination that the first collection of workouts includes a subsequent workout that follows the first respective workout in the ordered sequence, displaying, via the one or more display generation components, a first selectable object that is selectable to initiate the subsequent workout in the first collection of workouts; and in accordance with a determination that the first respective workout is a final workout in the first collection of workouts, displaying, via the one or more display generation components, a workout summary user interface, including concurrently displaying: a first workout metric that corresponds to the first respective workout but does not correspond to at least some of the other workouts in the first collection of workouts; and a second workout metric that is cumulative for the first collection of workouts.

In accordance with some embodiments, a computer system configured to communicate with one or more display generation components and one or more input devices, is described. The computer system comprising: means for receiving, via the one or more input devices, a first user input corresponding to a request to initiate a workout session corresponding to a first collection of workouts, wherein the first collection of workouts includes a plurality of workouts arranged in an ordered sequence, including a first workout and a second workout different from the first workout; means, in response to receiving the first user input, for displaying, via the one or more display generation components, visual content corresponding to a first respective workout of the plurality of workouts; and means, subsequent to displaying the visual content corresponding to the first respective workout, for: in accordance with a determination that the first collection of workouts includes a subsequent workout that follows the first respective workout in the ordered sequence, displaying, via the one or more display generation components, a first selectable object that is selectable to initiate the subsequent workout in the first collection of workouts; and in accordance with a determination that the first respective workout is a final workout in the first collection of workouts, displaying, via the one or more display generation components, a workout summary user interface, including concurrently displaying: a first workout metric that corresponds to the first respective workout but does not correspond to at least some of the other workouts in the first collection of workouts; and a second workout metric that is cumulative for the first collection of workouts.

In accordance with some embodiments, a computer program product, comprising one or more programs configured to be executed by one or more processors of a computer system that is in communication with one or more display generation components and one or more input devices, is described. The one or more programs including instructions for: receiving, via the one or more input devices, a first user input corresponding to a request to initiate a workout session corresponding to a first collection of workouts, wherein the first collection of workouts includes a plurality of workouts arranged in an ordered sequence, including a first workout and a second workout different from the first workout; in response to receiving the first user input, displaying, via the one or more display generation components, visual content corresponding to a first respective workout of the plurality of workouts; and subsequent to displaying the visual content corresponding to the first respective workout: in accordance with a determination that the first collection of workouts includes a subsequent workout that follows the first respective workout in the ordered sequence, displaying, via the one or more display generation components, a first selectable object that is selectable to initiate the subsequent workout in the first collection of workouts; and in accordance with a determination that the first respective workout is a final workout in the first collection of workouts, displaying, via the one or more display generation components, a workout summary user interface, including concurrently displaying: a first workout metric that corresponds to the first respective workout but does not correspond to at least some of the other workouts in the first collection of workouts; and a second workout metric that is cumulative for the first collection of workouts.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for providing and accessing workout content, thereby increasing the effectiveness, efficiency, and user satisfaction with such devices. Such methods and interfaces may complement or replace other methods for providing and accessing workout content.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3 is a block diagram of an exemplary multifunction device with a display and a touch-sensitive surface in accordance with some embodiments.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6GG illustrate exemplary user interfaces for providing and accessing workout content including workout plan content, in accordance with some embodiments.

FIG. 7 illustrates a flow diagram depicting a method for providing and accessing workout content, in accordance with some embodiments.

FIG. 8 illustrates a flow diagram depicting a method for providing and accessing workout content, in accordance with some embodiments.

FIG. 9 illustrates a flow diagram depicting a method for providing and accessing workout content, in accordance with some embodiments.

FIGS. 10A-10V illustrate exemplary user interfaces for providing and accessing workout content, in accordance with some embodiments.

FIG. 11 illustrates a flow diagram depicting a method for providing and accessing workout content, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for electronic devices that provide efficient methods and interfaces for providing and accessing workout content. Such techniques can reduce the cognitive burden on a user who provide and access workout content, thereby enhancing productivity. Further, such techniques can reduce processor and battery power otherwise wasted on redundant user inputs.

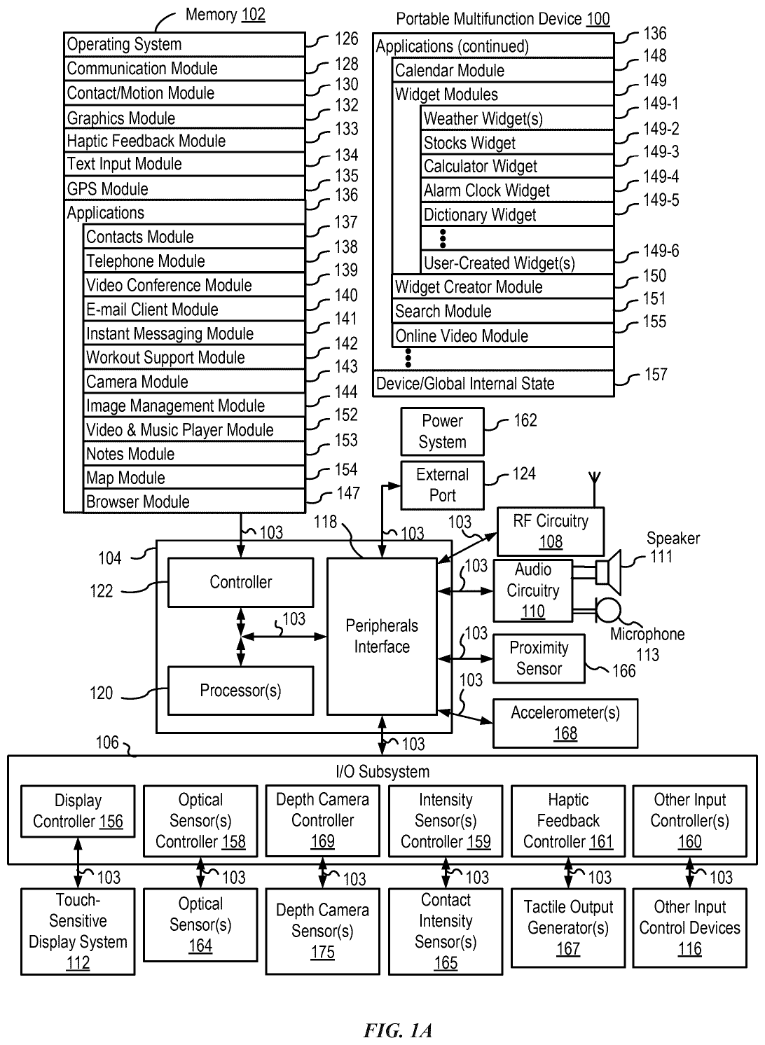

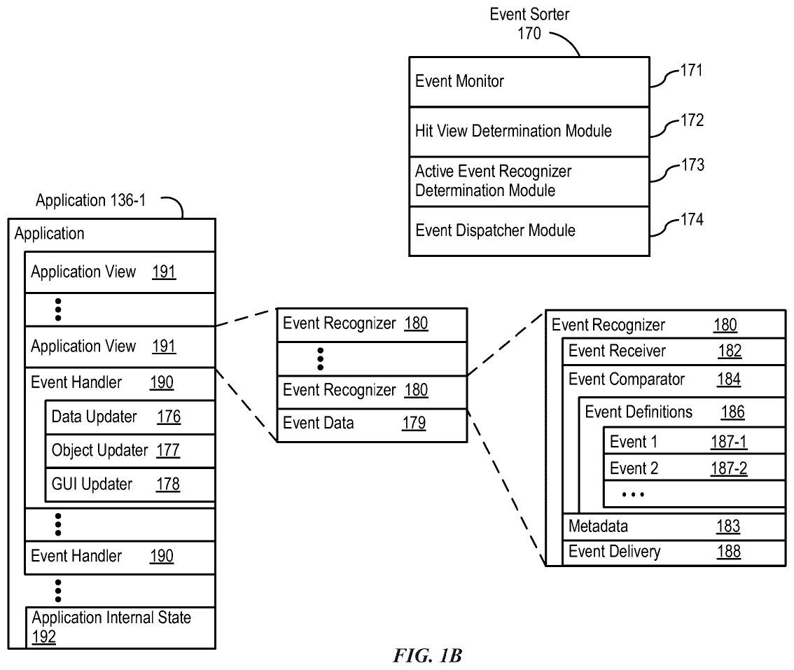

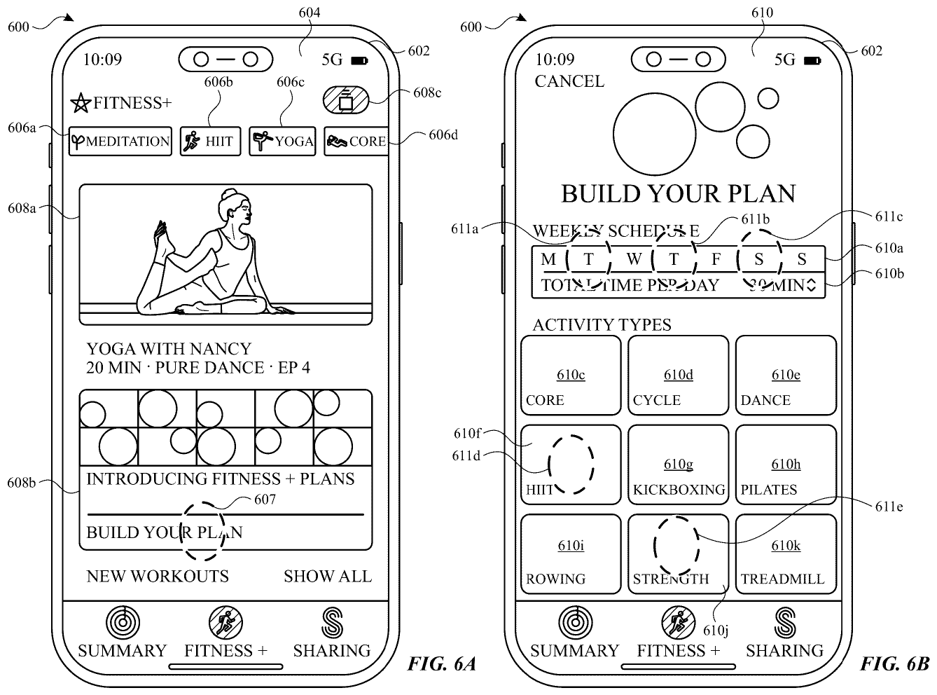

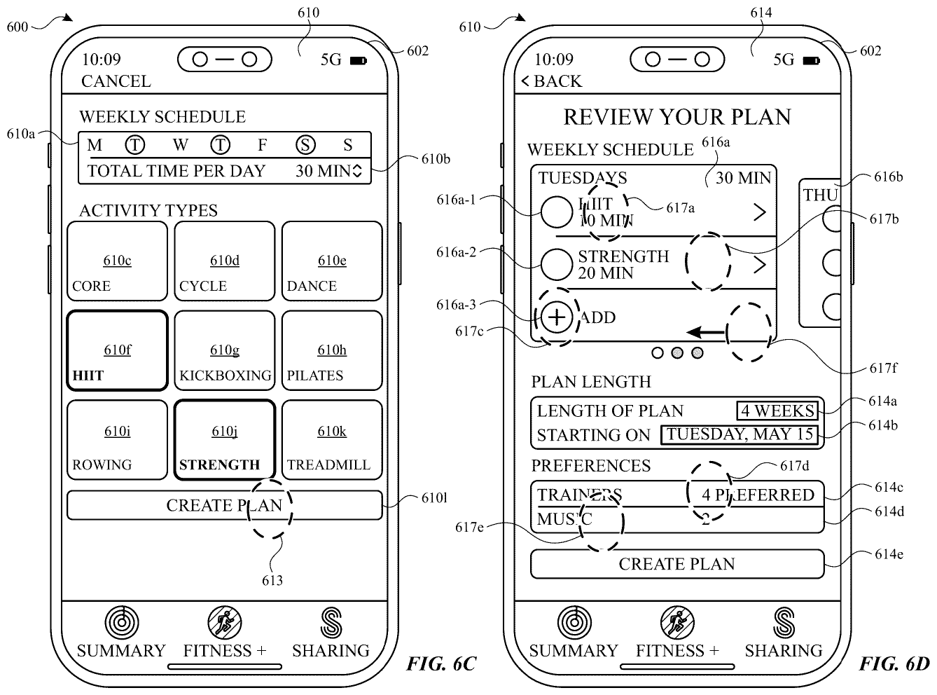

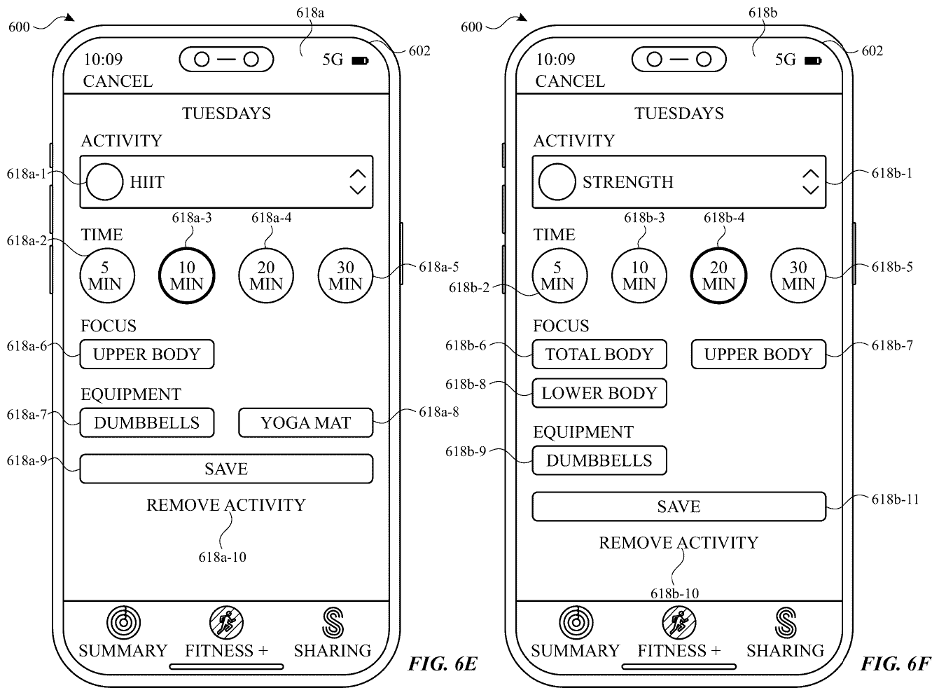

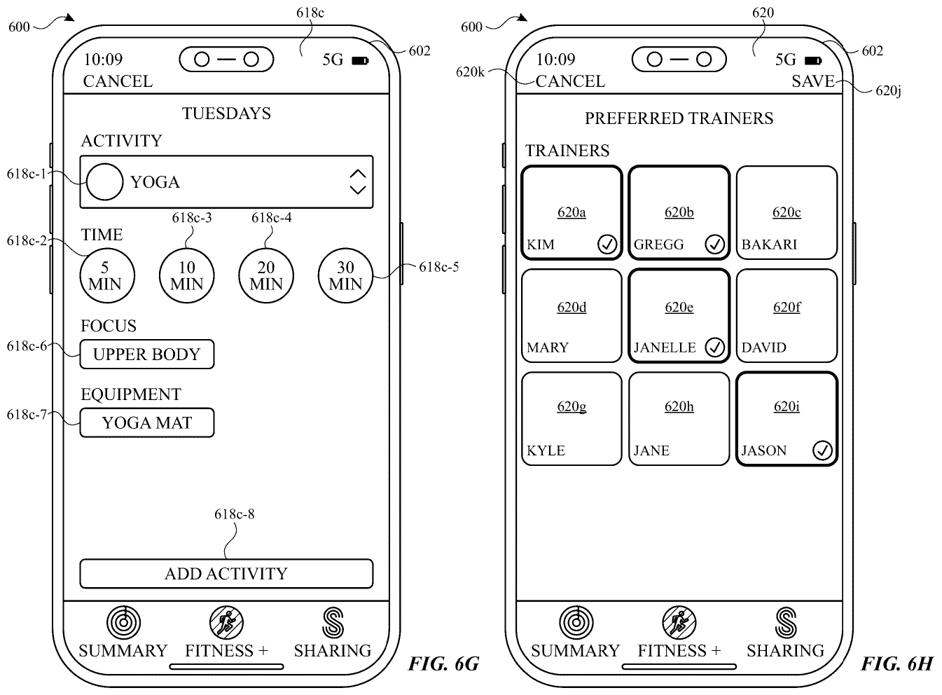

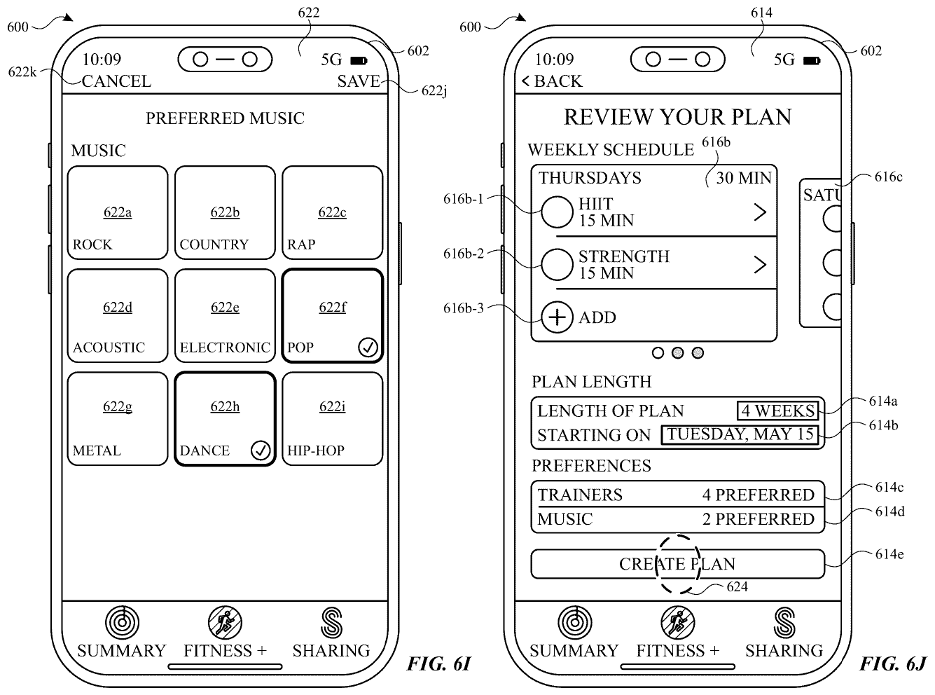

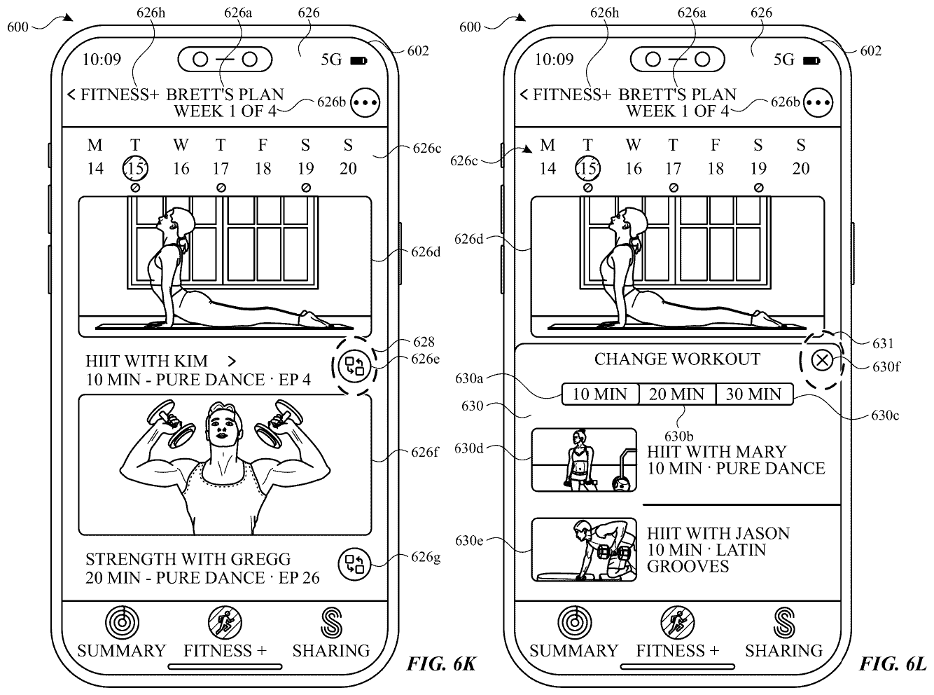

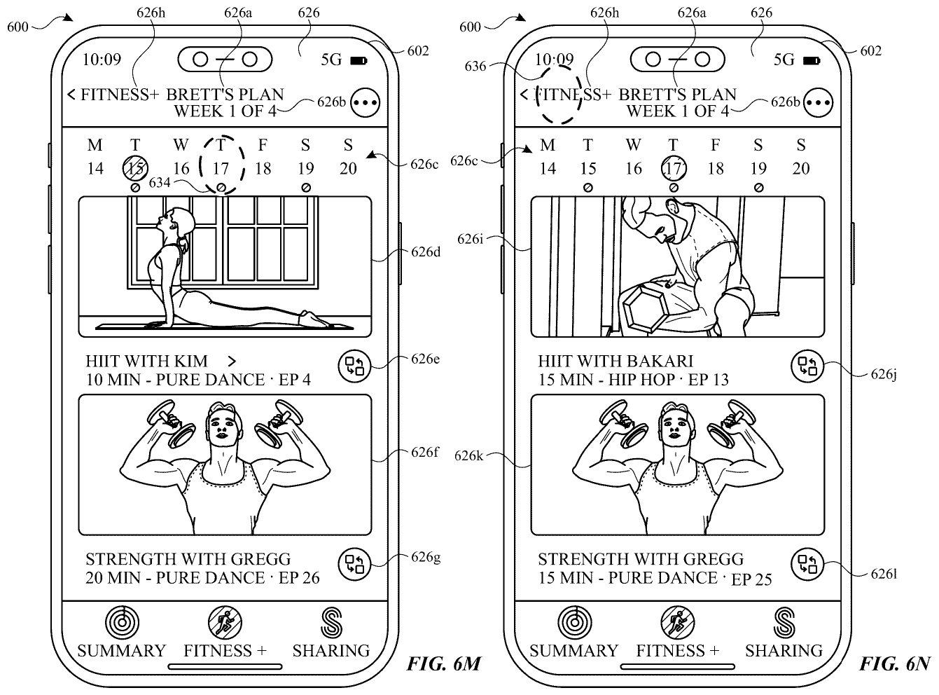

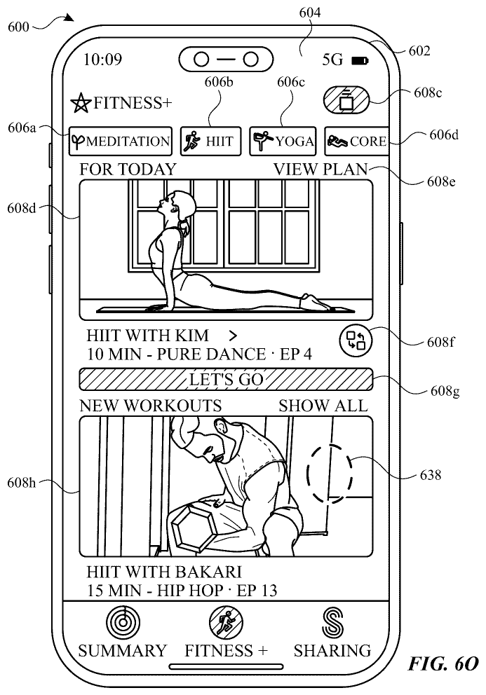

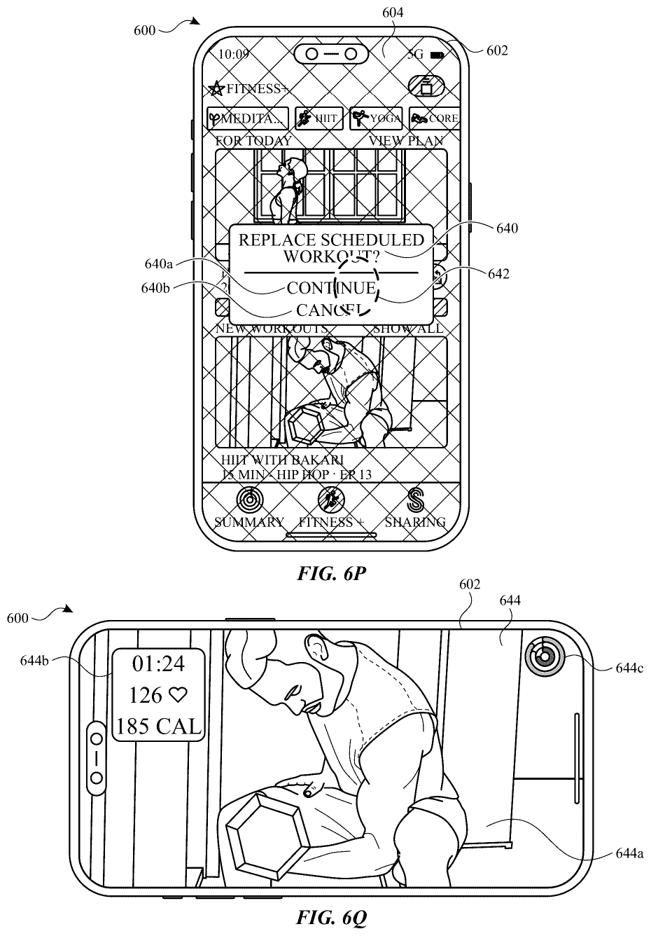

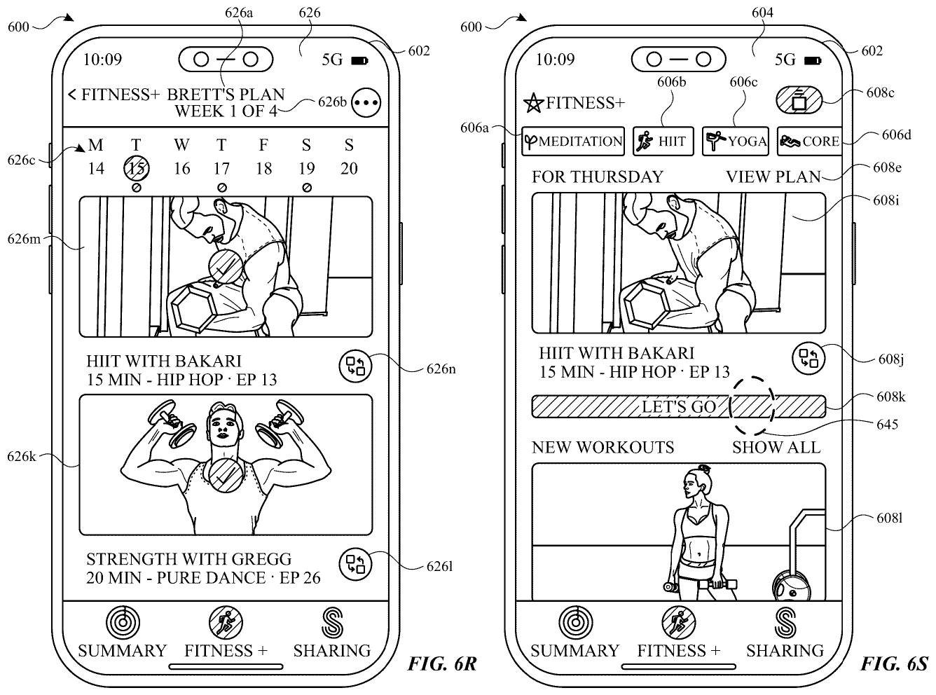

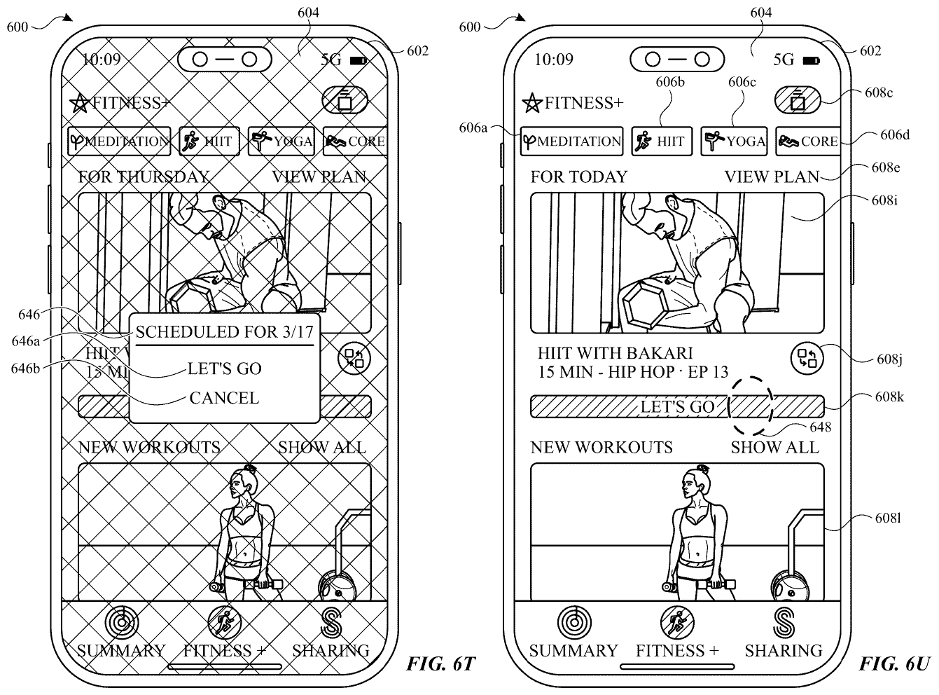

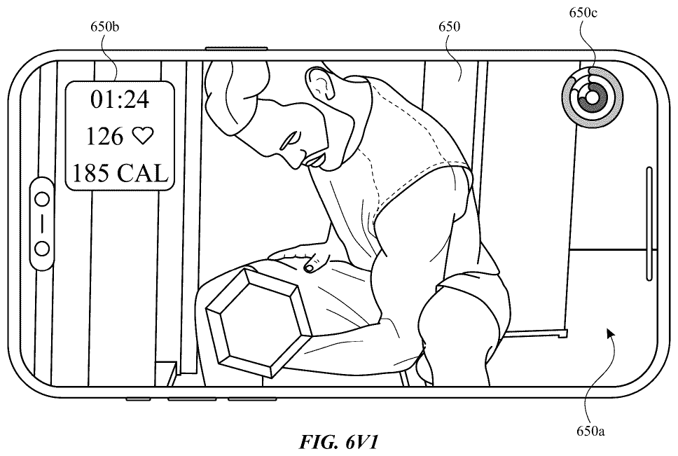

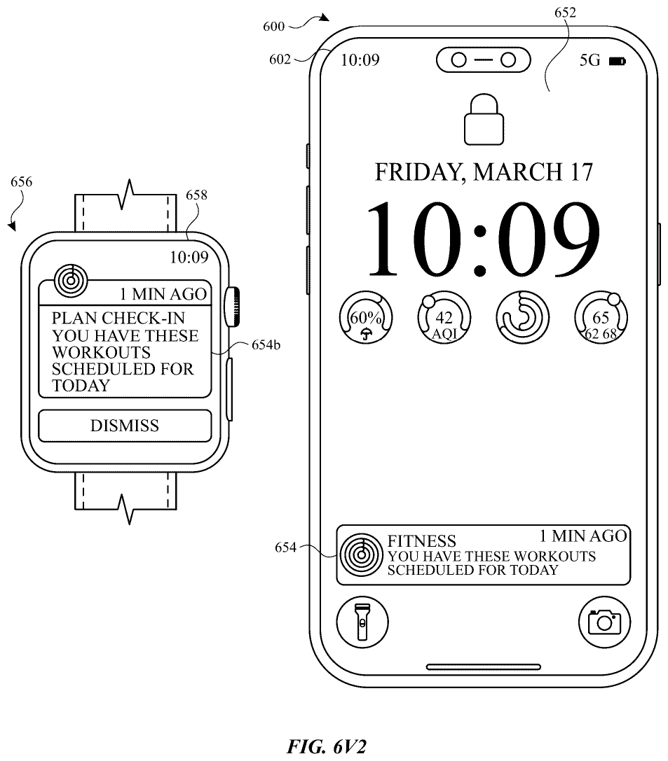

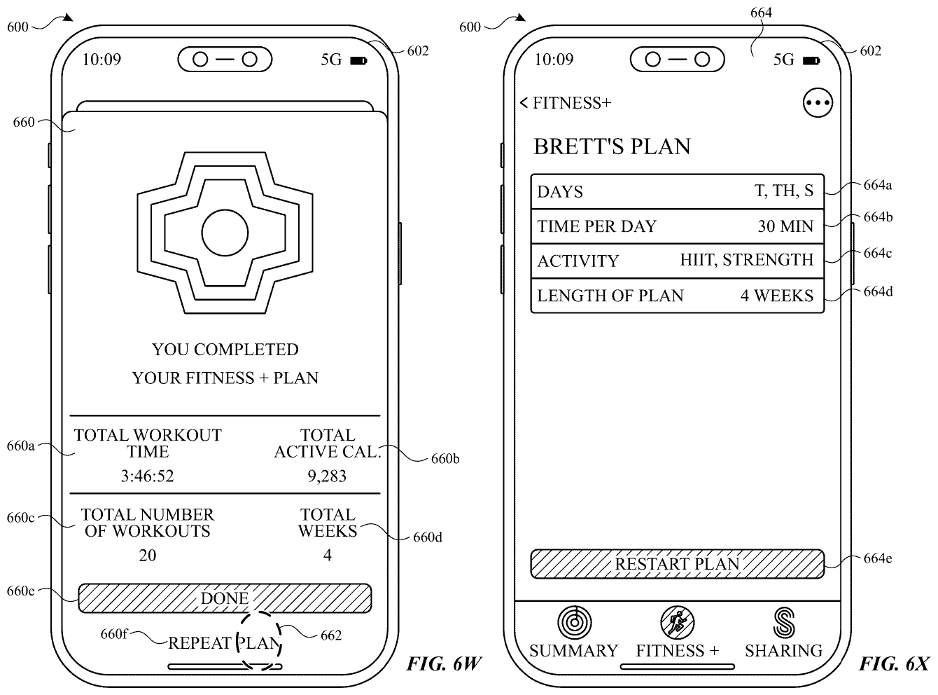

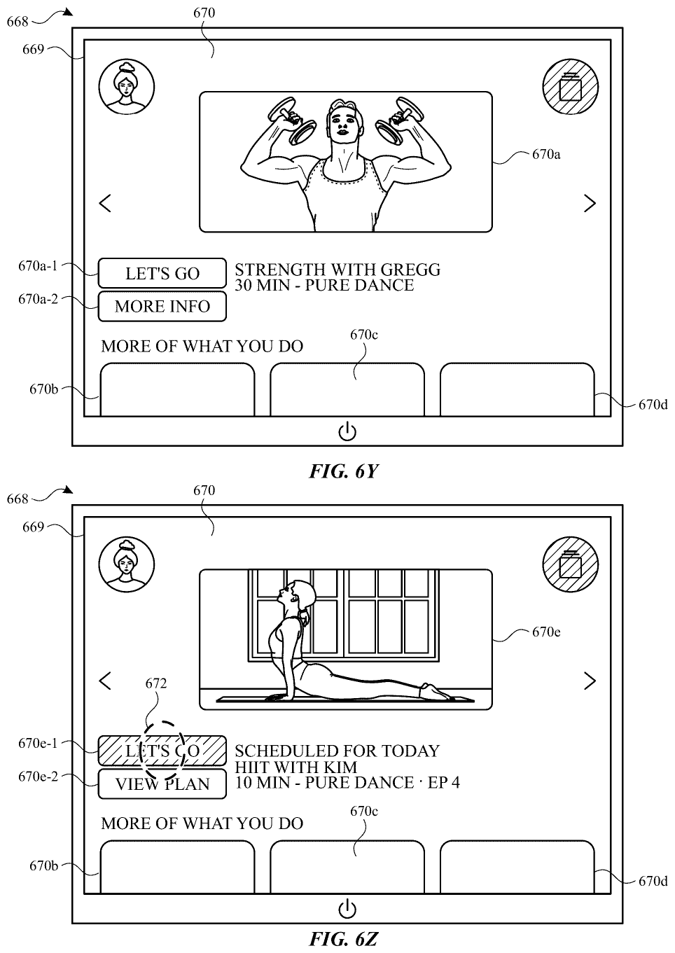

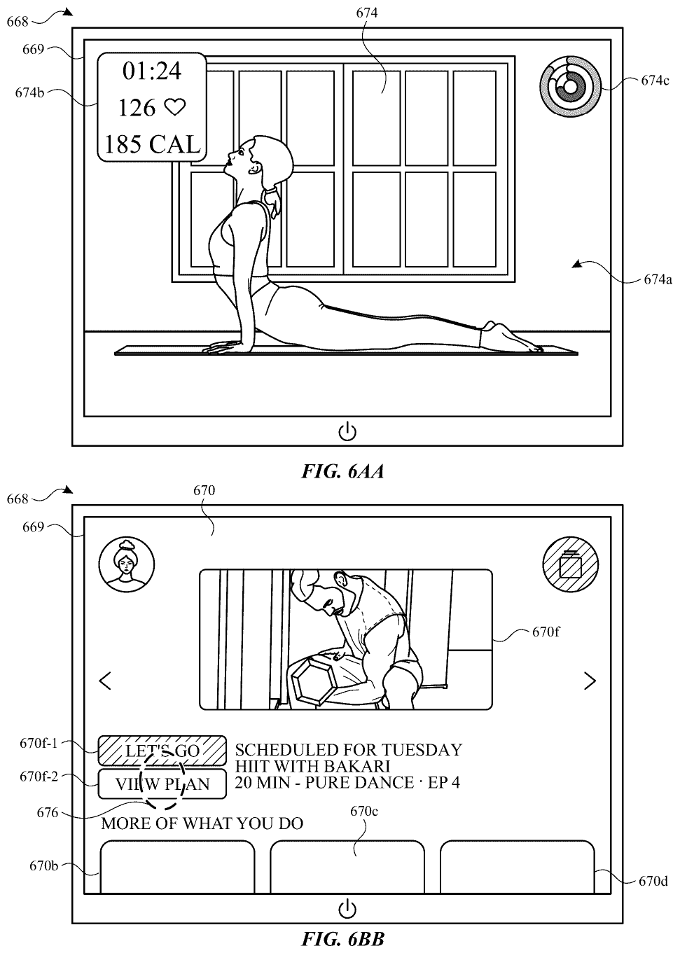

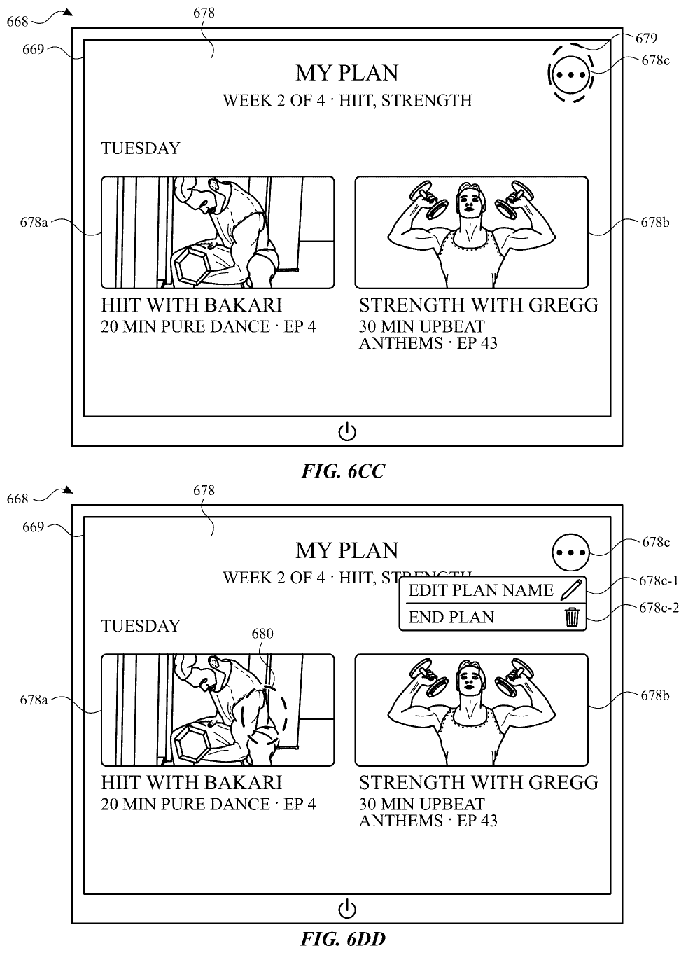

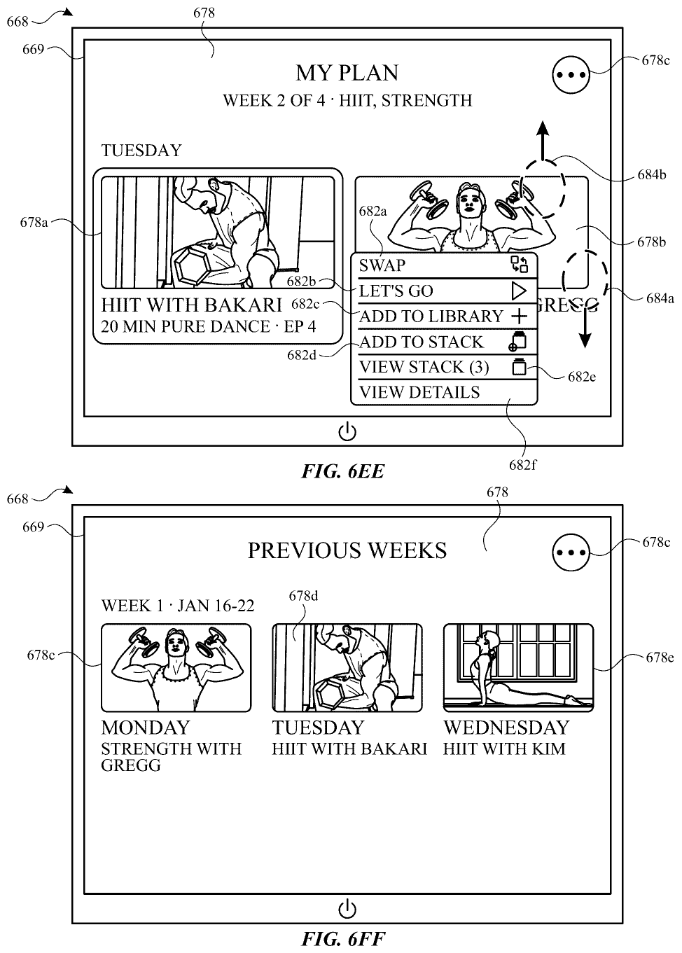

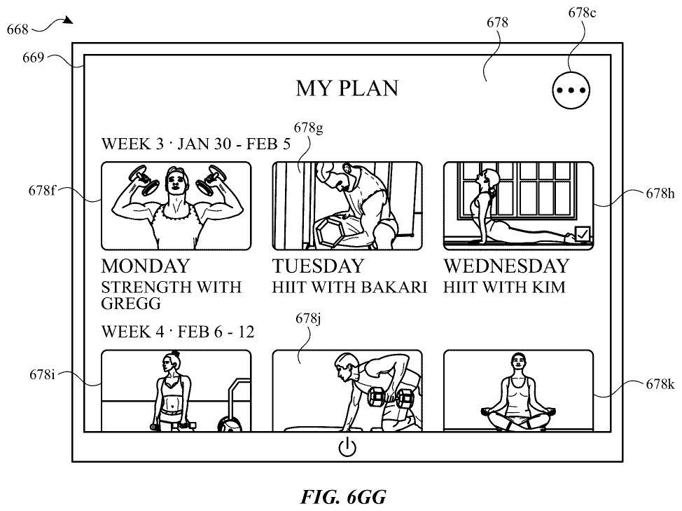

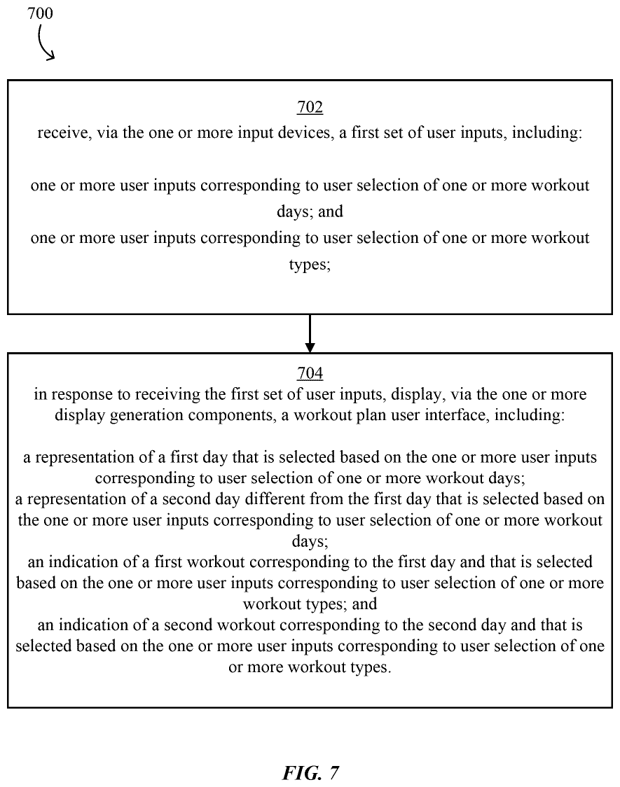

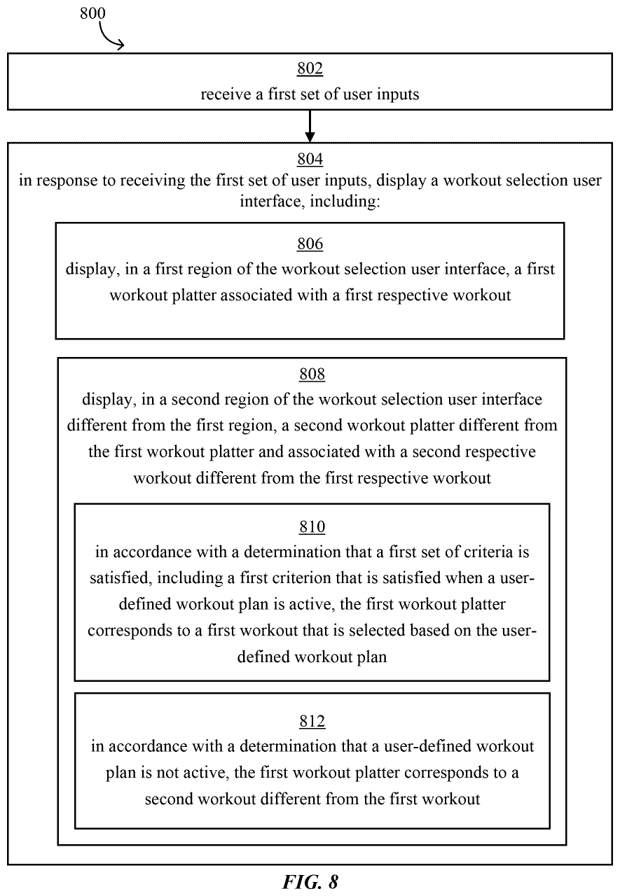

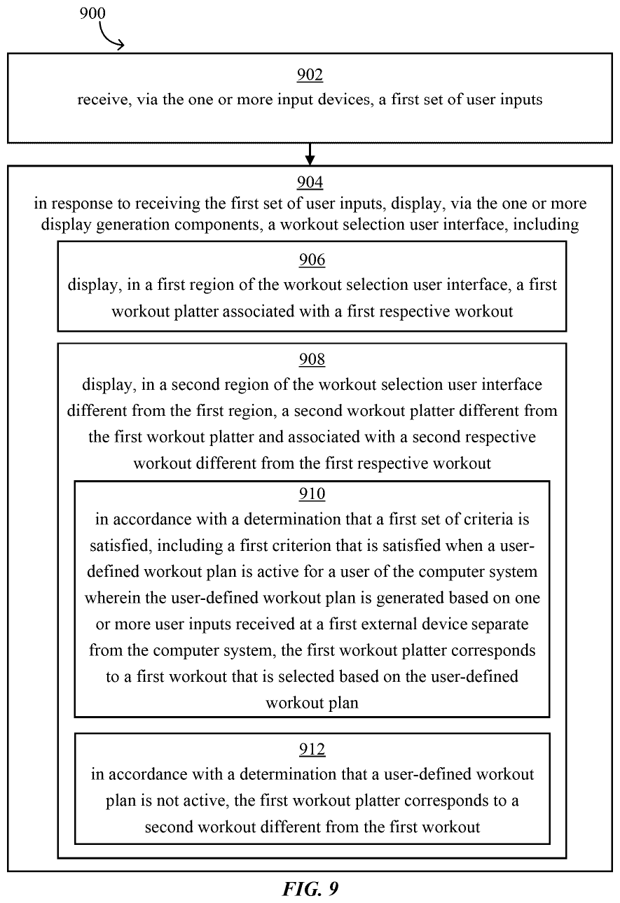

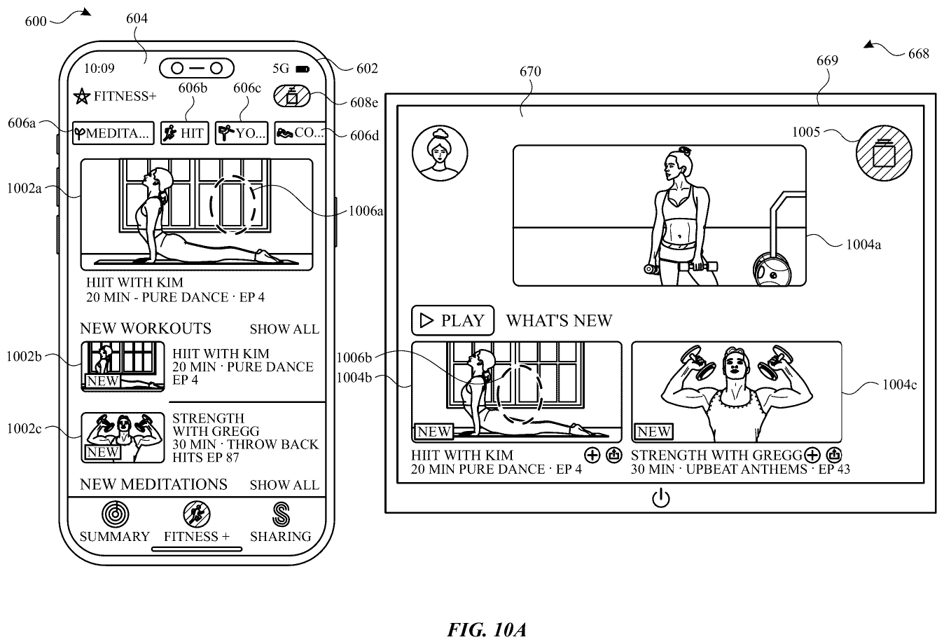

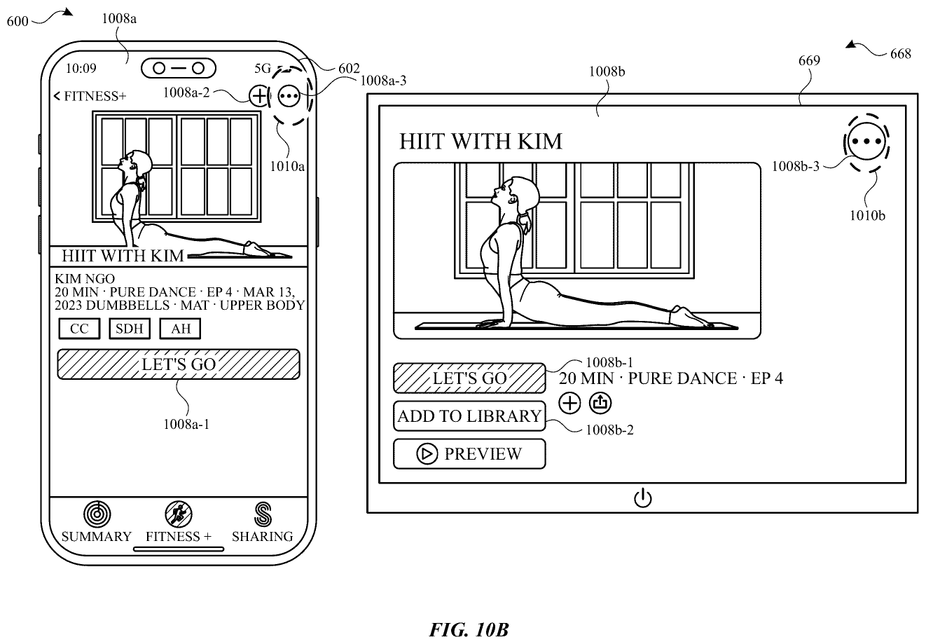

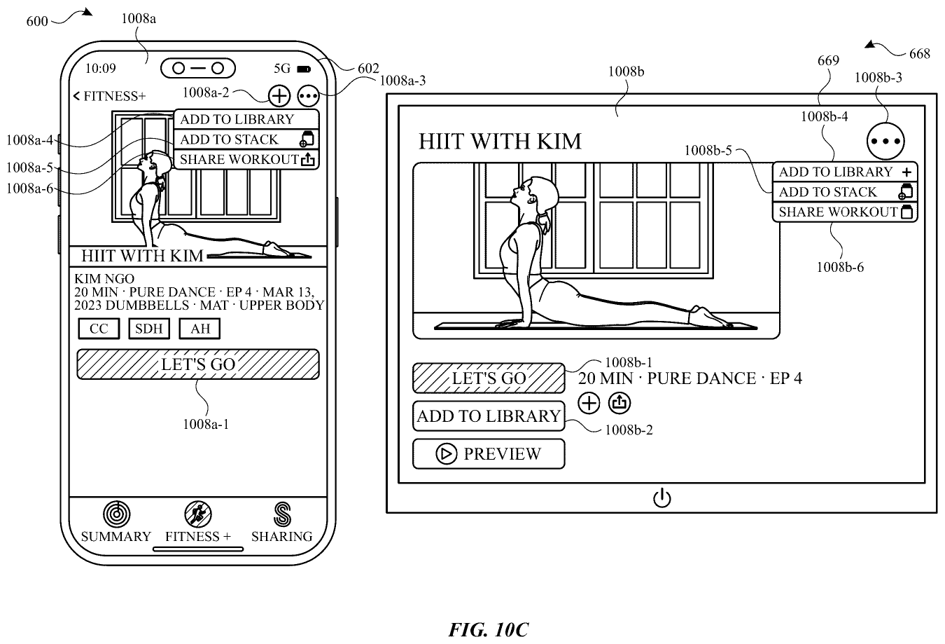

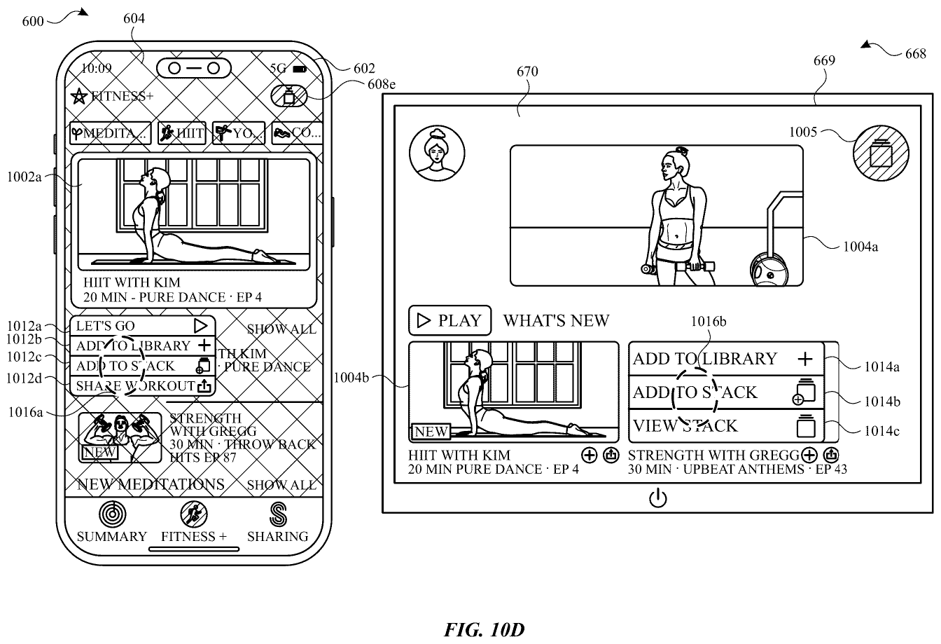

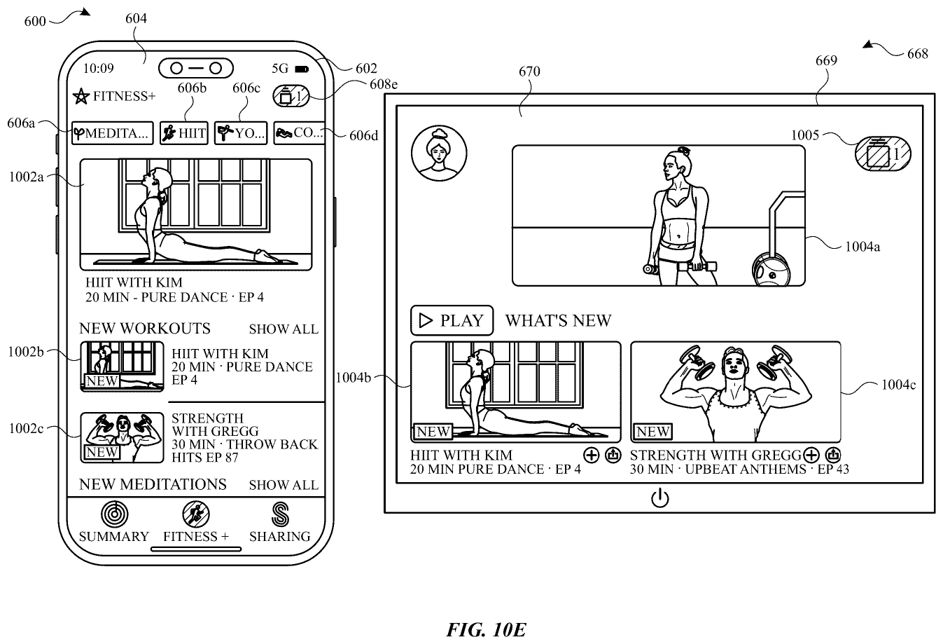

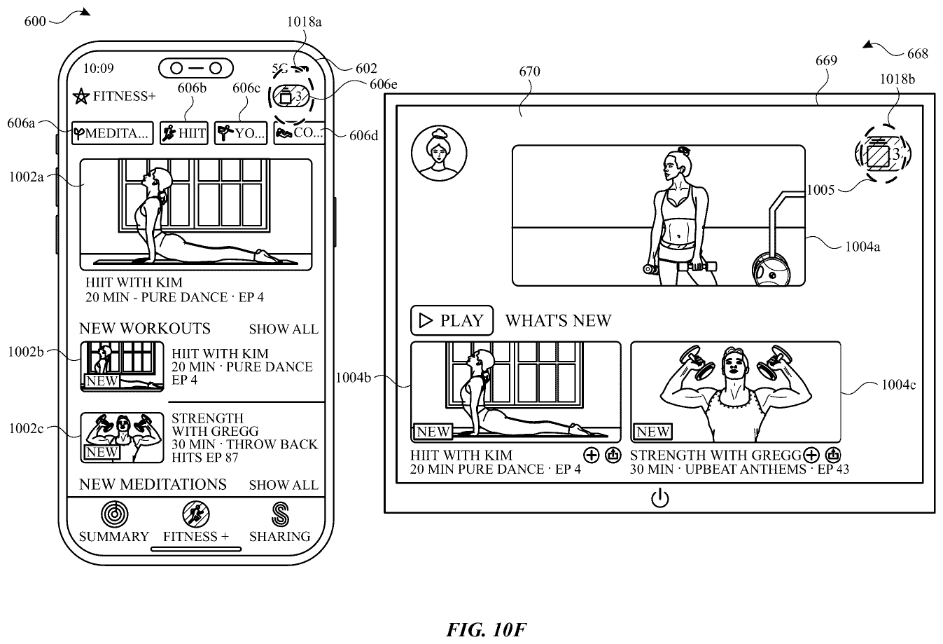

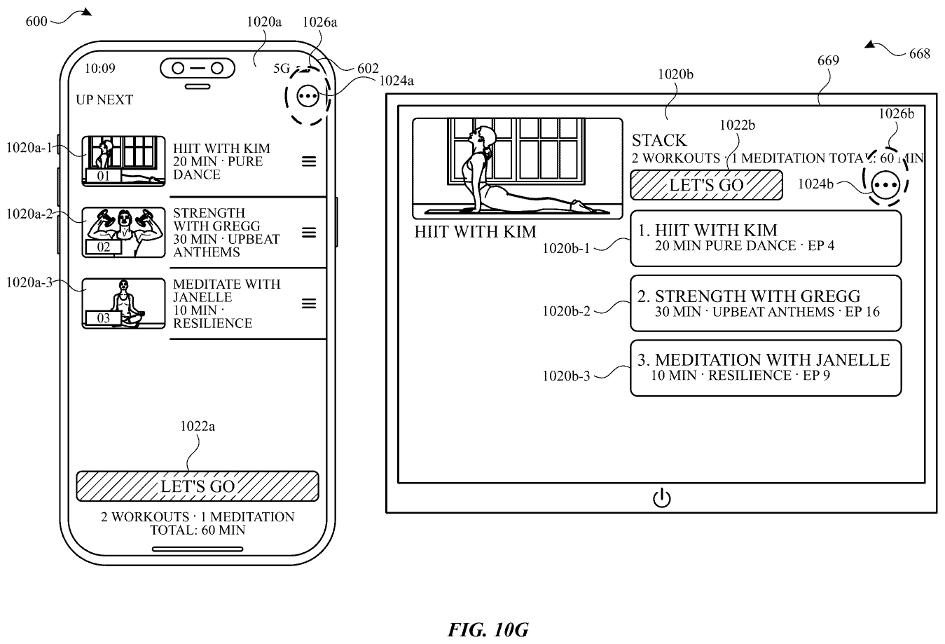

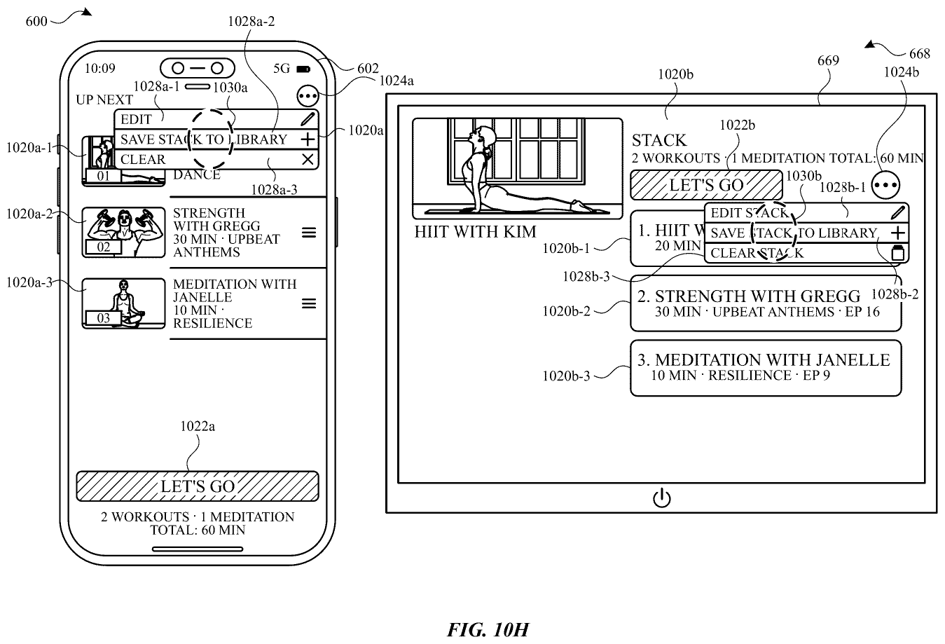

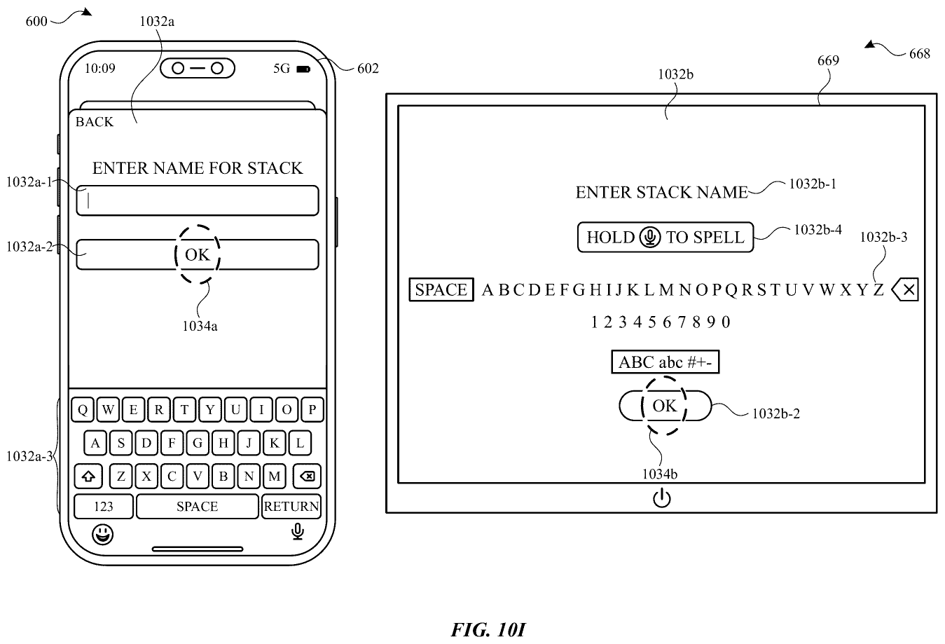

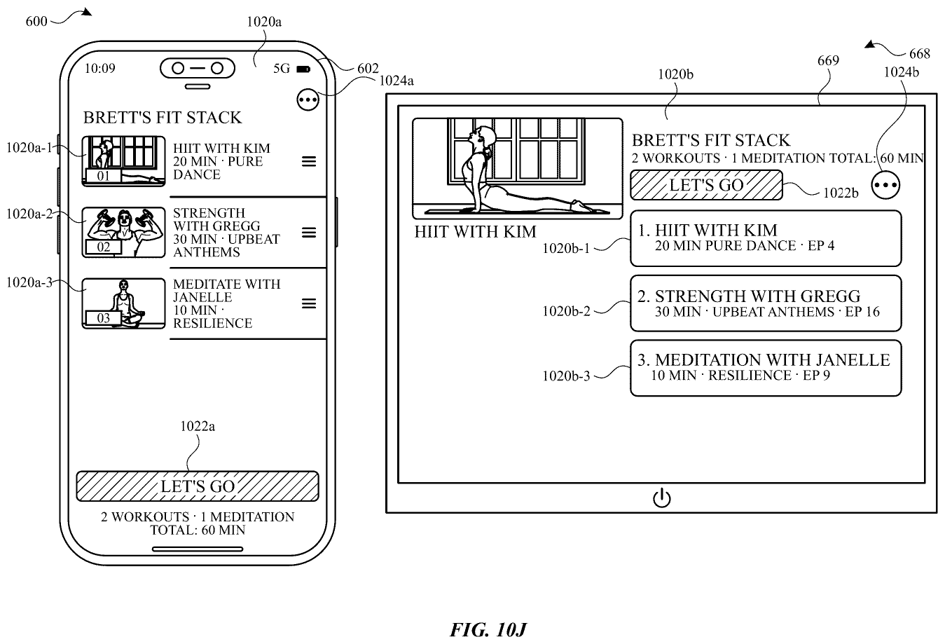

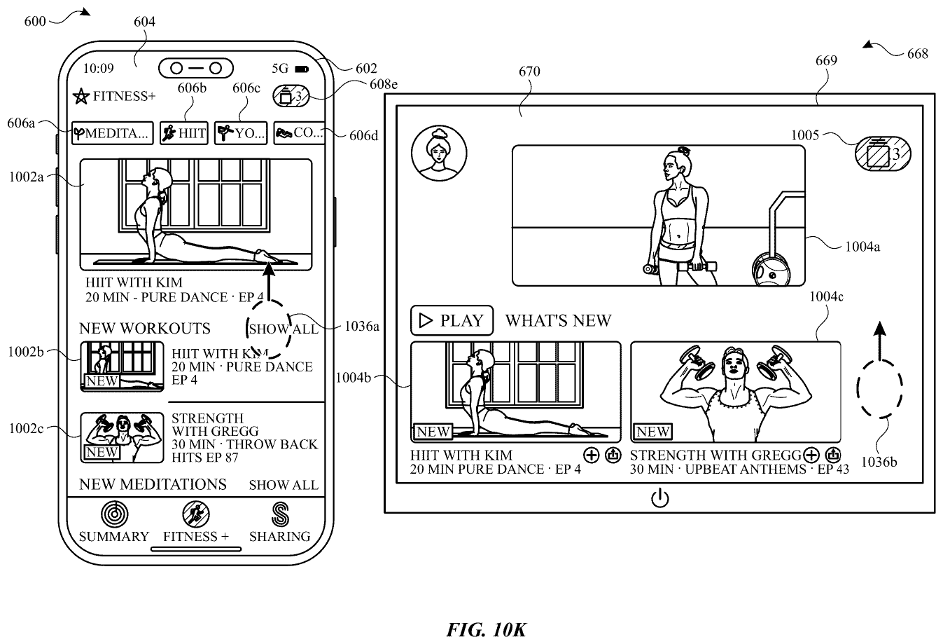

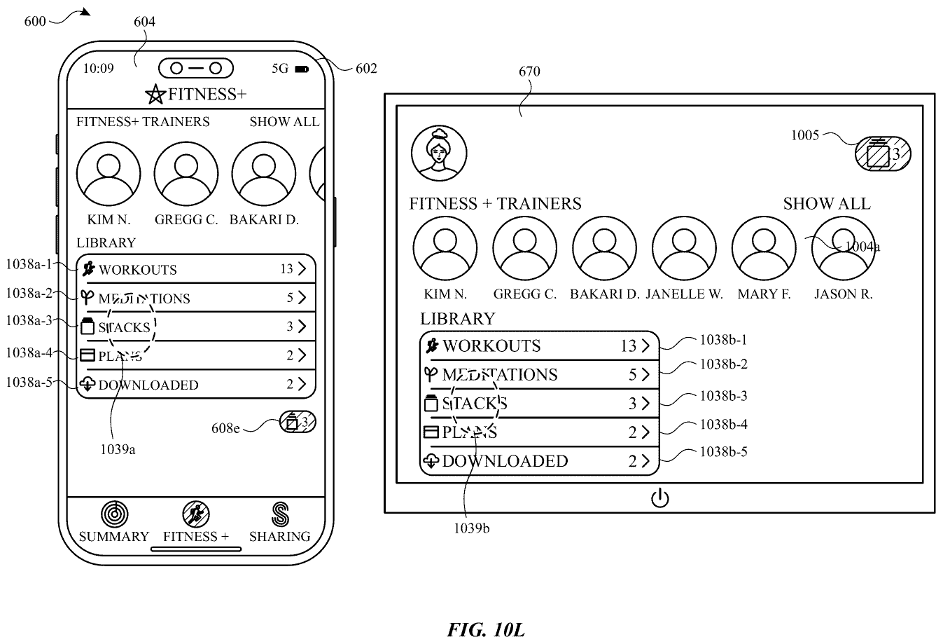

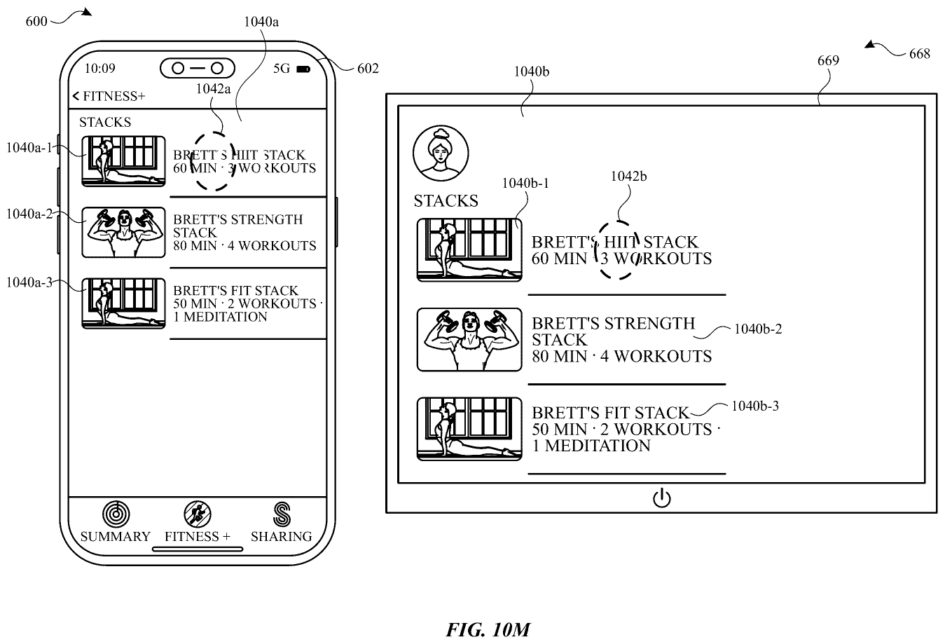

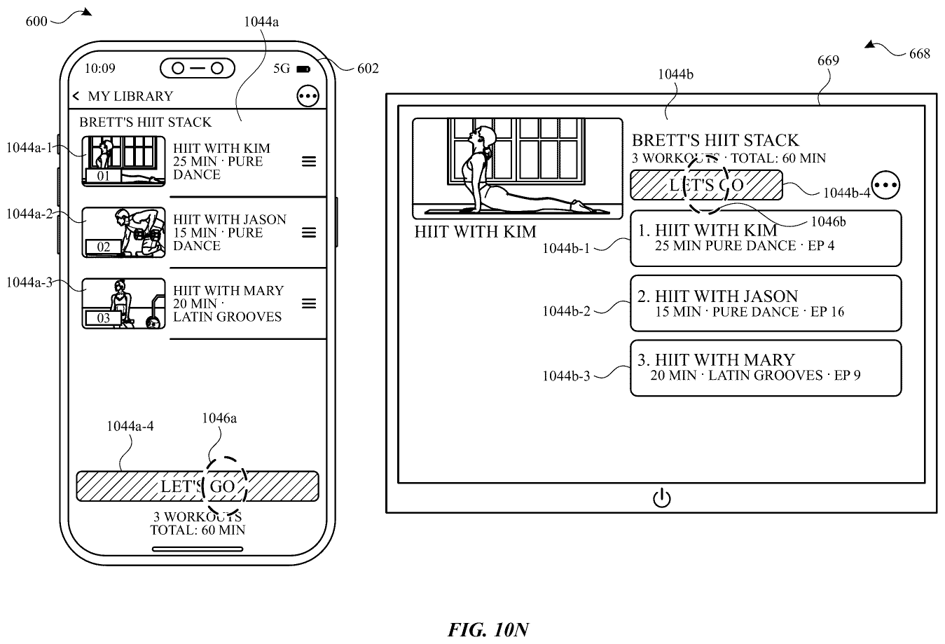

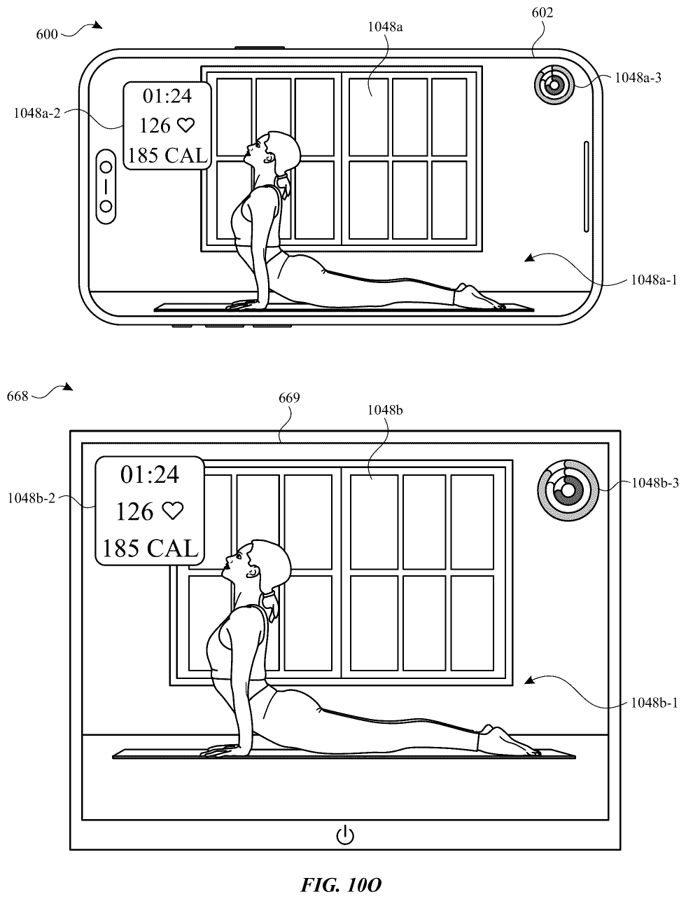

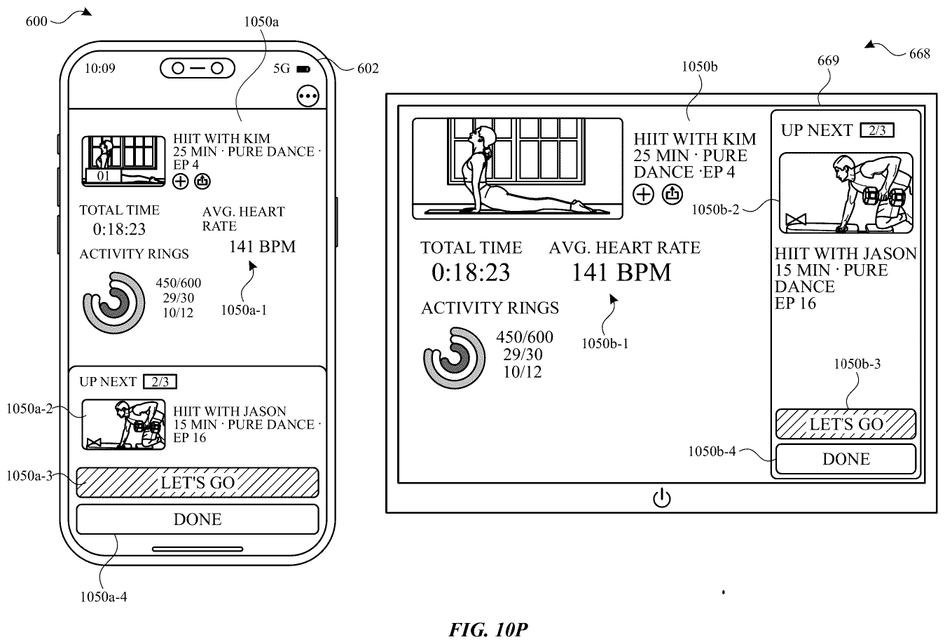

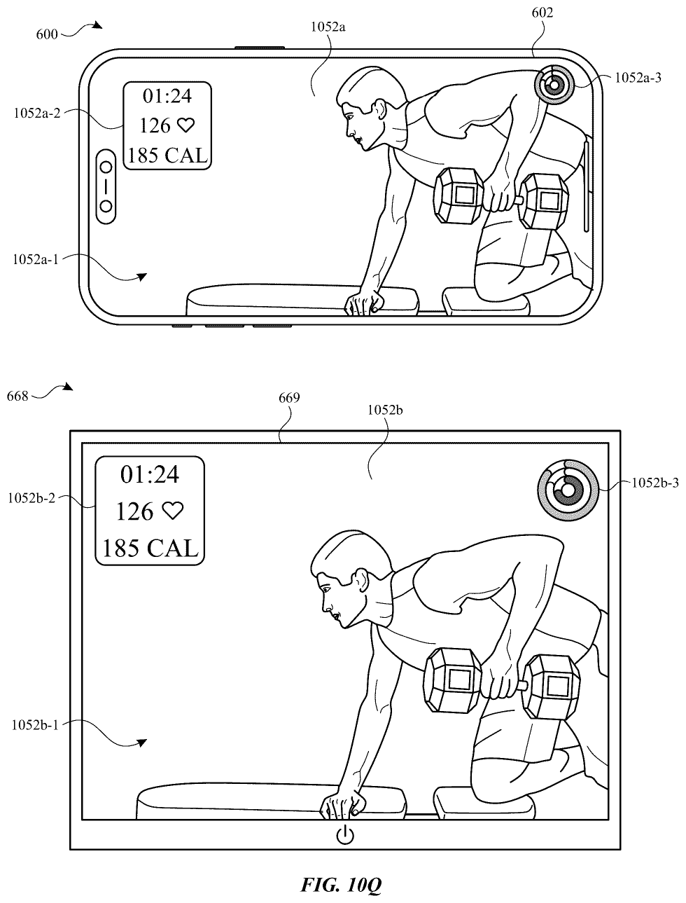

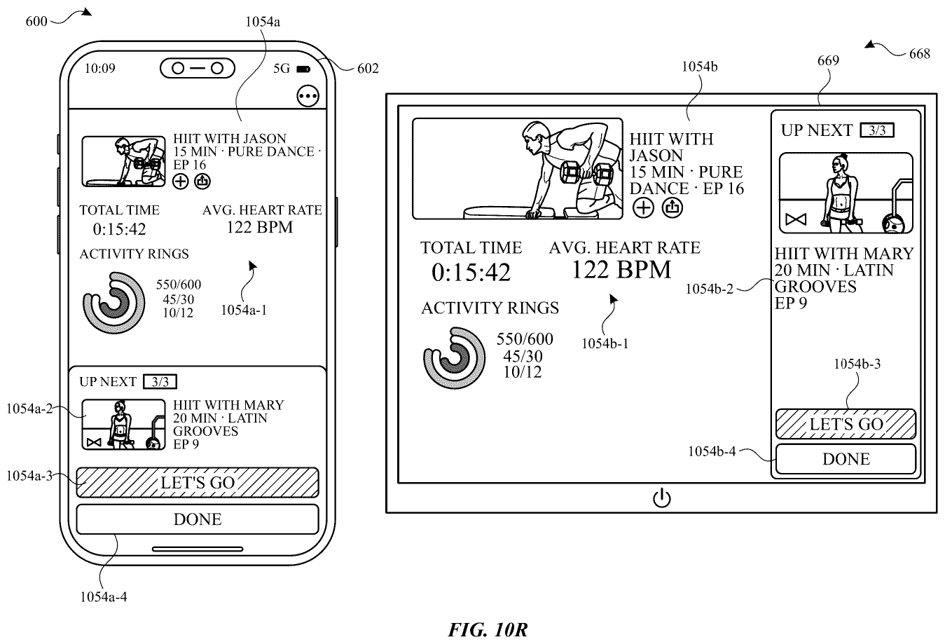

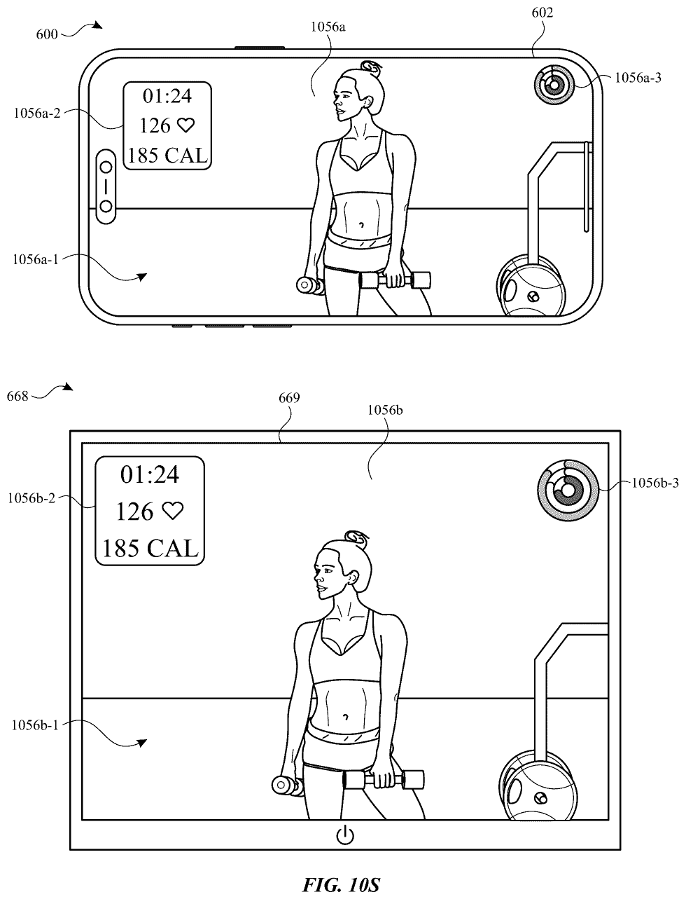

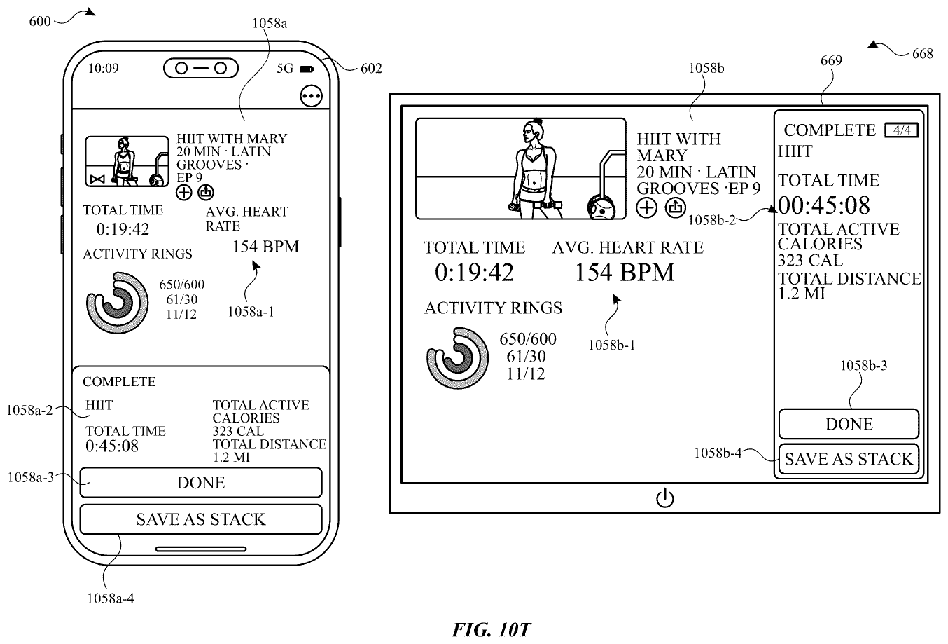

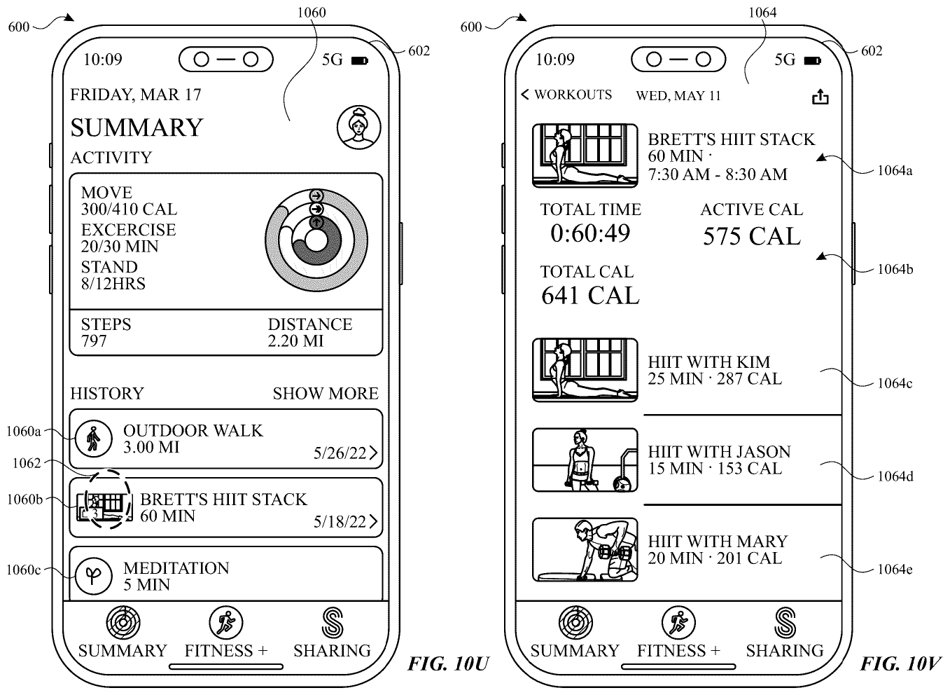

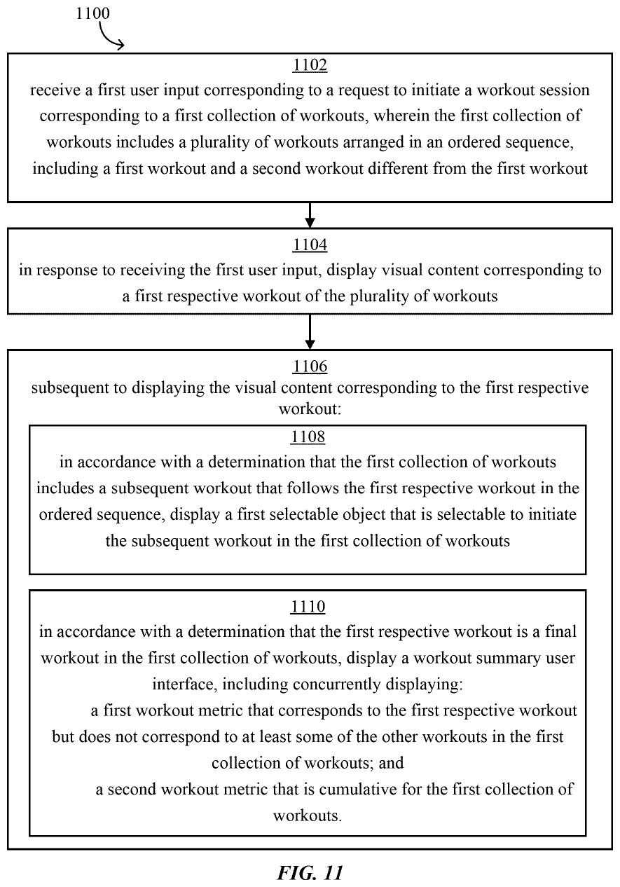

Below, FIGS. 1A-1B, 2, 3, 4A-4B, and 5A-5B provide a description of exemplary devices for performing the techniques for managing event notifications. FIGS. 6A-6GG illustrate exemplary user interfaces for providing and accessing workout content, in accordance with some embodiments. FIG. 7 is a flow diagram illustrating methods of providing and accessing workout content, in accordance with some embodiments. FIG. 8 is a flow diagram illustrating methods of providing and accessing workout content, in accordance with some embodiments. FIG. 9 is a flow diagram illustrating methods of providing and accessing workout content, in accordance with some embodiments. The user interfaces in FIGS. 6A-6GG are used to illustrate the processes described below, including the processes in FIGS. 7-9. FIGS. 10A-10V illustrate exemplary user interfaces for providing and accessing workout content, in accordance with some embodiments. FIG. 11 is a flow diagram illustrating methods of providing and accessing workout content, in accordance with some embodiments. The user interfaces in FIGS. 10A-10V are used to illustrate the processes described below, including the processes in FIG. 11.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component. The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage mediums), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

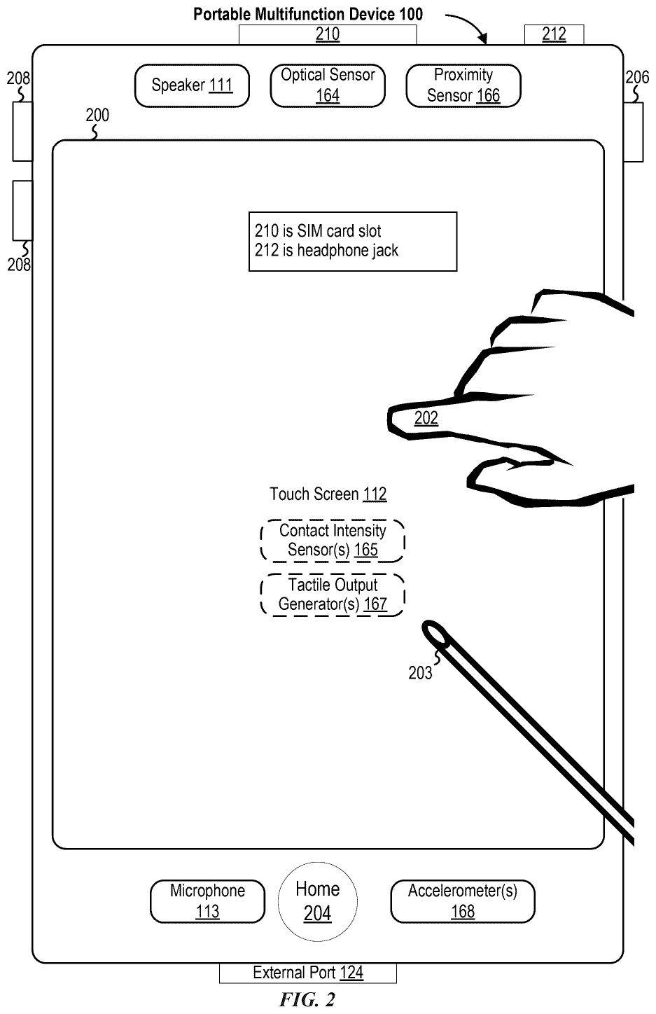

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both cars) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is a part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CMOS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

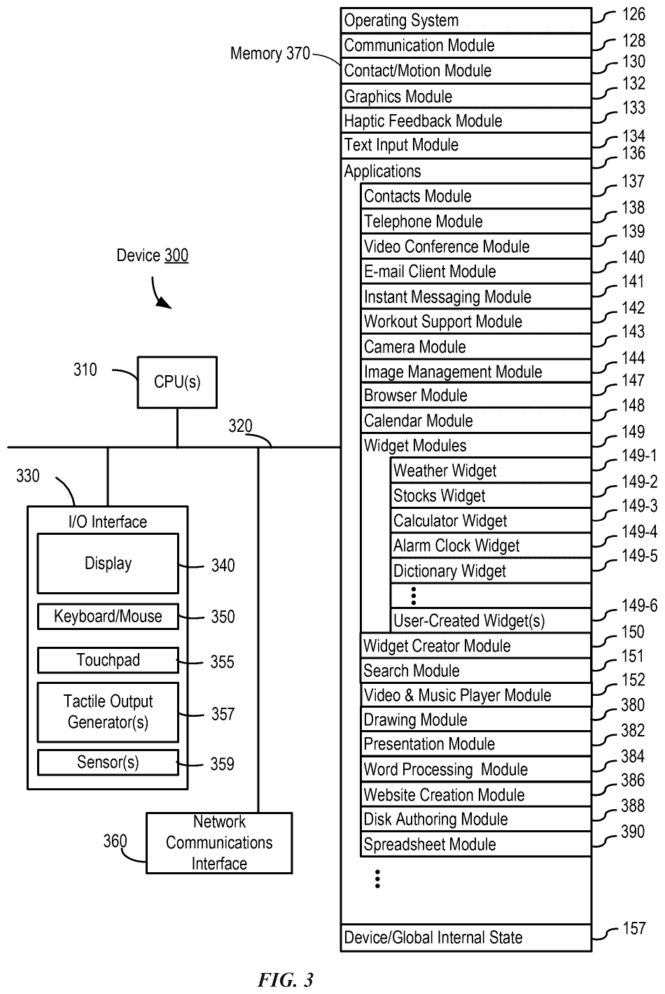

In some embodiments, the software components stored in memory 102 include operating system 126, communication module (or set of instructions) 128, contact/motion module (or set of instructions) 130, graphics module (or set of instructions) 132, text input module (or set of instructions) 134, Global Positioning System (GPS) module (or set of instructions) 135, and applications (or sets of instructions) 136. Furthermore, in some embodiments, memory 102 (FIG. 1A) or 370 (FIG. 3) stores device/global internal state 157, as shown in FIGS. 1A and 3. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, IOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts 137, e-mail 140, IM 141, browser 147, and any other application that needs text input).

GPS module 135 determines the location of the device and provides this information for use in various applications (e.g., to telephone 138 for use in location-based dialing; to camera 143 as picture/video metadata; and to applications that provide location-based services such as weather widgets, local yellow page widgets, and map/navigation widgets).

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone 138, video conference module 139, e-mail 140, or IM 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript file. In some embodiments, a widget includes an XML (Extensible Markup Language) file and a JavaScript file (e.g., Yahoo! Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3 is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3 is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Attention is now directed towards embodiments of user interfaces that are, optionally, implemented on, for example, portable multifunction device 100.

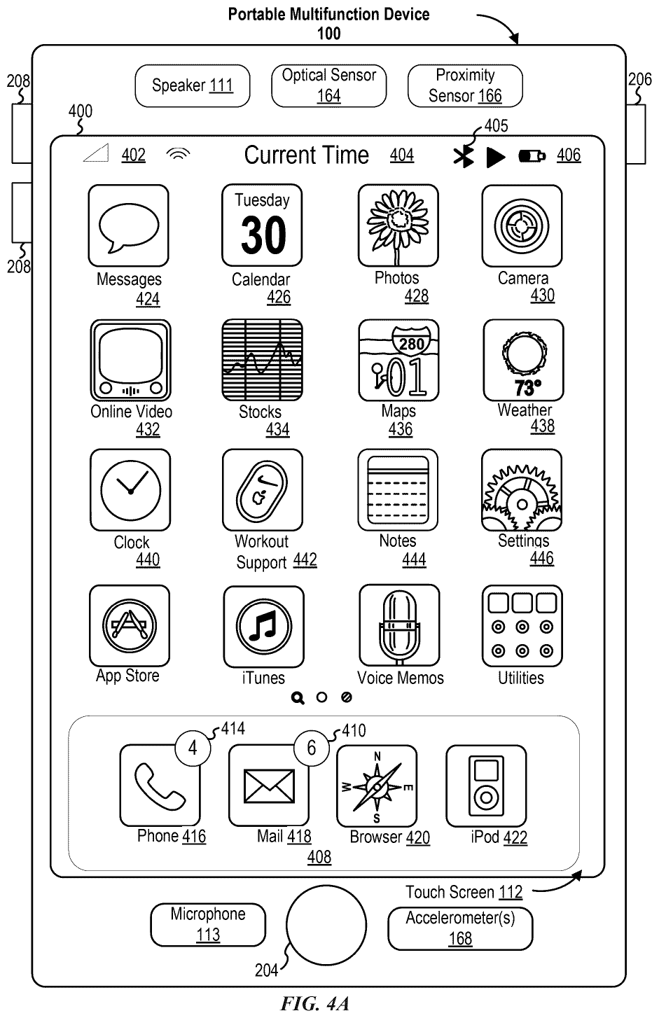

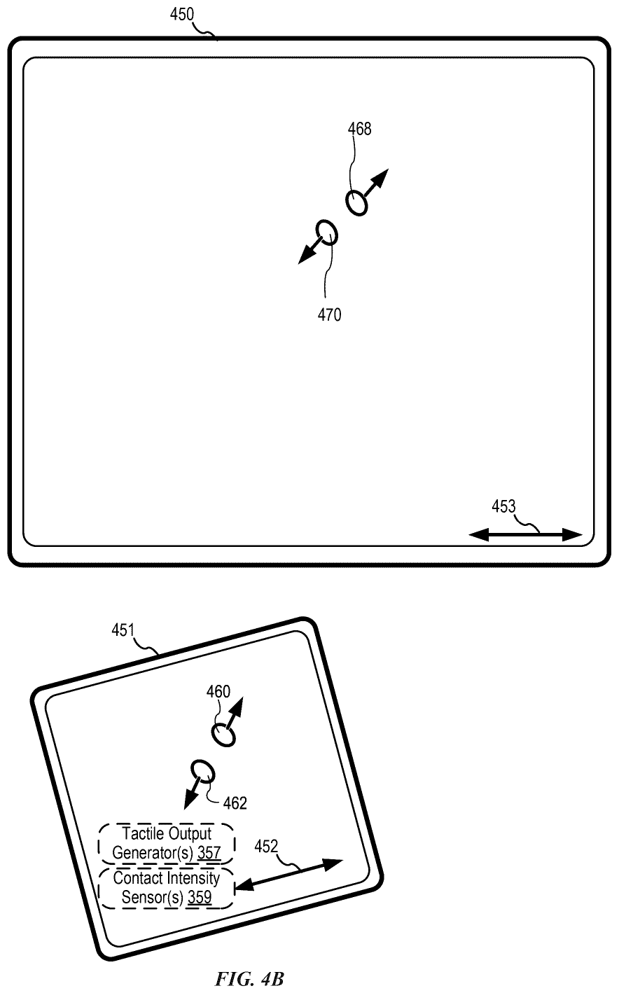

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3) with a touch-sensitive surface 451 (e.g., a tablet or touchpad 355, FIG. 3) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., 451 in FIG. 4B) has a primary axis (e.g., 452 in FIG. 4B) that corresponds to a primary axis (e.g., 453 in FIG. 4B) on the display (e.g., 450). In accordance with these embodiments, the device detects contacts (e.g., 460 and 462 in FIG. 4B) with the touch-sensitive surface 451 at locations that correspond to respective locations on the display (e.g., in FIG. 4B, 460 corresponds to 468 and 462 corresponds to 470). In this way, user inputs (e.g., contacts 460 and 462, and movements thereof) detected by the device on the touch-sensitive surface (e.g., 451 in FIG. 4B) are used by the device to manipulate the user interface on the display (e.g., 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

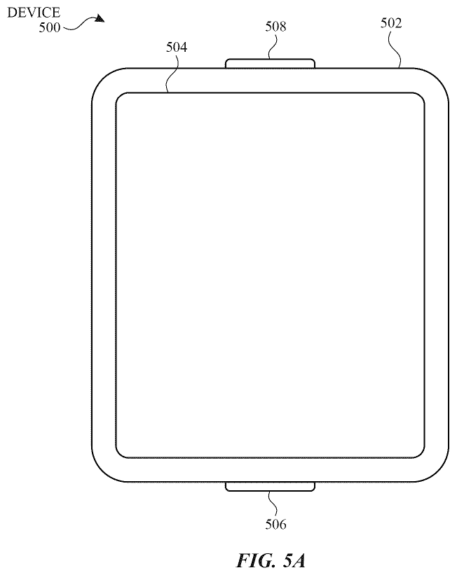

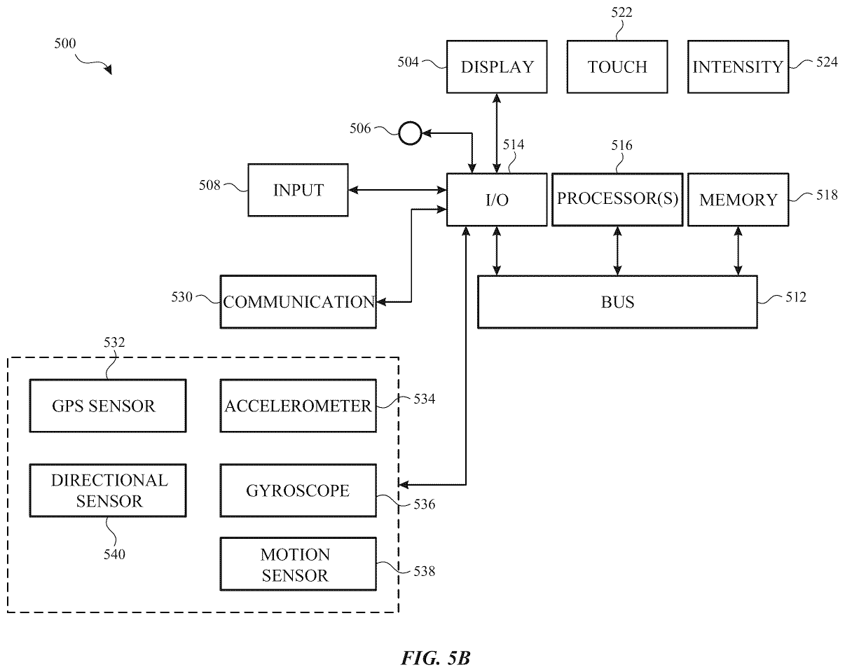

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3. Device 500 has bus 512 that operatively couples I/O section 514 with one or more computer processors 516 and memory 518. I/O section 514 can be connected to display 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage mediums, for storing computer-executable instructions, which, when executed by one or more computer processors 516, for example, can cause the computer processors to perform the techniques described below, including processes 700, 800, 900, and 1100 (FIGS. 7-9 and 10). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3 or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

As used herein, an “installed application” refers to a software application that has been downloaded onto an electronic device (e.g., devices 100, 300, and/or 500) and is ready to be launched (e.g., become opened) on the device. In some embodiments, a downloaded application becomes an installed application by way of an installation program that extracts program portions from a downloaded package and integrates the extracted portions with the operating system of the computer system.

As used herein, the terms “open application” or “executing application” refer to a software application with retained state information (e.g., as part of device/global internal state 157 and/or application internal state 192). An open or executing application is, optionally, any one of the following types of applications:
an active application, which is currently displayed on a display screen of the device that the application is being used on;
a background application (or background processes), which is not currently displayed, but one or more processes for the application are being processed by one or more processors; and
a suspended or hibernated application, which is not running, but has state information that is stored in memory (volatile and non-volatile, respectively) and that can be used to resume execution of the application.

As used herein, the term “closed application” refers to software applications without retained state information (e.g., state information for closed applications is not stored in a memory of the device). Accordingly, closing an application includes stopping and/or removing application processes for the application and removing state information for the application from the memory of the device. Generally, opening a second application while in a first application does not close the first application. When the second application is displayed and the first application ceases to be displayed, the first application becomes a background application.

In some embodiments, the computer system is in a locked state or an unlocked state. In the locked state, the computer system is powered on and operational but is prevented from performing a predefined set of operations in response to user input. The predefined set of operations optionally includes navigation between user interfaces, activation or deactivation of a predefined set of functions, and activation or deactivation of certain applications. The locked state can be used to prevent unintentional or unauthorized use of some functionality of the computer system or activation or deactivation of some functions on the computer system. In some embodiments, in the unlocked state, the computer system is powered on and operational and is not prevented from performing at least a portion of the predefined set of operations that cannot be performed while in the locked state. When the computer system is in the locked state, the computer system is said to be locked. When the computer system is in the unlocked state, the computer is said to be unlocked. In some embodiments, the computer system in the locked state optionally responds to a limited set of user inputs, including input that corresponds to an attempt to transition the computer system to the unlocked state or input that corresponds to powering the computer system off.

Attention is now directed towards embodiments of user interfaces (“UI”) and associated processes that are implemented on an electronic device, such as portable multifunction device 100, device 300, or device 500.

FIGS. 6A-6GG illustrate exemplary user interfaces for providing and accessing workout content including workout plan content, in accordance with some embodiments. The user interfaces in these figures are used to illustrate the processes described below, including the processes in FIGS. 7, 8, and 9.

FIG. 6A illustrates electronic device 600, which is a smart phone with touch-sensitive display 602. At FIG. 6A, electronic device 600 displays workout selection user interface 604. Workout selection user interface 604 includes filter options 606a-606d that are selectable to filter available workout options based on workout type. Workout selection user interface 604 also includes workout representation 608a that is selectable to initiate a first workout. Workout selection user interface 604 also includes option 608b, that is selectable to initiate a process for building a user-defined workout plan. At FIG. 6A, electronic device 600 detects user input 607 (e.g., a tap input) corresponding to selection of option 608b.

At FIG. 6B, in response to user input 607, electronic device 600 displays user interface 610. User interface 610 includes a plurality of options for building a user-defined workout plan. User interface 610 includes day options 610a that allows a user to define one or more days of the week on which the user wishes to schedule a workout. User interface 610 also includes time option 610b that allows a user to specify how many minutes and/or hours per workout day the user would like to work out. User interface 610 also includes workout type options 610c-610k that allow a user to define which types of workouts the user would like to include in his or her workout plan. At FIG. 6B, electronic device 600 detects user inputs 611a-611c.

At FIG. 6C, in response to user inputs 611a-611e, electronic device 600 modifies display of user interface 610. In FIG. 6C, user interface 610 has been modified to indicate that the user has selected Tuesdays, Thursdays, and Saturdays as workout days for the user-defined workout plan, and the user has selected HIIT (option 610f) and Strength (option 610j) as workout types for the user-defined workout plan. At FIG. 6C, electronic device 600 detects user input 613 corresponding to selection of option 610l.

At FIG. 6D, in response to use input 613, electronic device 600 displays user interface 614. User interface 614 displays a proposed workout plan based on the user selections in user interface 610. User interface 614 displays a proposed weekly schedule that includes day representation 616a, representative of Tuesdays, and day representation 616b, representative of Thursdays (e.g., based on user selection of Tuesdays and Thursdays as workout days in user interface 610). Day representation 616a includes first object 616a-1, which indicates that a 10 minute HIIT workout will be scheduled every Tuesday, and object 616a-2, which indicates that a 20-minute strength workout will be scheduled every Tuesday (e.g., based on user selection of HIIT and strength as workout types in user interface 610, and user specification of 30 workout minutes per workout day in user interface 610). Day representation 616a also includes object 616a-3 that is selectable to indicate a user request to add one or more workouts to Tuesdays in the user-defined workout plan. User interface 614 also includes length of plan information 614a that indicates a proposed duration for the user-defined workout plan and is selectable to modify a total duration of the user-defined workout plan; start date information 614b that indicates a proposed start date for the user-defined workout plan and is selectable to modify the start date; trainer preference object 614c that is selectable to identify one or more preferred trainers for the user-defined workout plan; and object 614d that is selectable to identify one or more preferred music types for the user-defined workout plan. At FIG. 6D, electronic device 600 detects user inputs 617a, 617b, 617c, 617d, 617e, and 617f. Each of these user inputs will be described below.

At FIG. 6E, in response to detecting user input 617a corresponding to selection of option 616a-1 in FIG. 6D, electronic device 600 displays user interface 618a, which provides one or more options for modifying the proposed HIIT workout currently proposed for Tuesdays. User interface 618a includes option 618a-1, which is selectable to change a workout type (e.g., workout modality) for the proposed Tuesday workout. User interface 618a also includes duration options 618a-2, 618a-3, 618a-4, 618a-5 that are selectable to modify a duration for the proposed Tuesday workout. User interface 618a also includes option 618a-6 that is selectable to specify a focus area for the proposed Tuesday workout. User interface 618a also includes equipment preferences 618a-7, 618a-8 that are selectable to indicate that the user would like to schedule workouts for Tuesdays that involve certain types of workout equipment. User interface 618a also includes option 618a-9 that is selectable to save user preferences for the proposed Tuesday HIIT workout, and option 618a-10 that is selectable to delete the proposed Tuesday HIIT workout.

At FIG. 6F, in response to detecting user input 617b corresponding to selection of option 616a-2 in FIG. 6D, electronic device 600 displays user interface 618b, which provides one or more options for modifying the proposed strength workout currently proposed for Tuesdays. User interface 618b includes option 618b-1, which is selectable to change a workout type (e.g., workout modality) for the proposed Tuesday workout. User interface 618b also includes duration options 618b-2, 618b-3, 618b-4, 618b-5 that are selectable to modify a duration for the proposed Tuesday workout. User interface 618b also includes options 618b-6, 618b-7, 618b-8 that are selectable to specify different focus areas for the proposed Tuesday workout (e.g., to select workouts that focus on the selected focus area). User interface 618b also includes equipment preference 618b-9, that is selectable to indicate that the user would like to schedule workouts for Tuesdays that involve certain types of workout equipment. User interface 618b also includes option 618b-11 that is selectable to save user preferences for the proposed Tuesday strength workout, and option 618a-10 that is selectable to delete the proposed Tuesday strength workout.

At FIG. 6G, in response to detecting user input 617c corresponding to selection of option 616a-33 in FIG. 6D, electronic device 600 displays user interface 618c, which provides one or more options for adding a new workout for Tuesdays in the user-defined workout plan. User interface 618c includes option 618c-1 which is selectable to define a workout type (e.g., workout modality) for the new Tuesday workout. User interface 618c also includes duration options 618c-2, 618c-3, 618c-4, 618c-5 that are selectable to define a duration for the proposed Tuesday workout. User interface 618c also includes option 618c-6 that is selectable to specify a focus area for the proposed Tuesday workout. User interface 618c also includes equipment preference 618c-7 that is selectable to indicate that the user would like to schedule workouts for Tuesdays that involve certain types of workout equipment. In some embodiments equipment preferences displayed in user interface 618c change based on the workout modality selected by the user in option 618c-1. User interface 618c also includes option 618c-8 that is selectable to add a new workout for Tuesdays in the user-defined workout plan.

At FIG. 6H, in response to detecting user input 617d corresponding to selection of option 614c in FIG. 6D, electronic device 600 displays user interface 620, which includes a plurality of trainer options 620a-620i for selection by the user. In user interface 620, a user can specify certain trainers that should be given higher priority and/or higher preference when the user-defined workout plan is generated. In some embodiments, workouts selected for the user-defined workout plan only include the selected trainers. In some embodiments, workouts selected for the user-defined workout plan give higher weight and/or preference to the selected trainers, but do not include exclusively workouts by the selected trainers. At FIG. 6H, the user has specified four trainers (Kim, Gregg, Janelle, and Jason) that the user prefers.

At FIG. 6I, in response to detecting user input 617e corresponding to selection of option 614d in FIG. 6D, electronic device 600 displays user interface 622, which includes a plurality of music options 622-622i for selection by the user. In user interface 622, a user can specify certain types of music that should be given higher priority and/or higher preference when the user-defined workout plan is generated. In some embodiments, workouts selected for the user-defined workout plan only include workouts that correspond to the selected music types. In some embodiments, workouts selected for the user-defined workout plan give higher weight and/or preference to the selected music types, but do not include exclusively workouts that correspond to the selected workout types. At FIG. 6I, the user has specified two types of music (Dance and Pop) that the user prefers.

At FIG. 6J, in response to detecting user input 617f (e.g., a swipe right input) on day representation 616a in FIG. 6D, electronic device 600 displays day representation 616a and day representation 616b translate to the left such that day representation 616b is now visible within user interface 614, and day representation 616c (corresponding to Saturdays) is also visible within user interface 614. Day representation 616b corresponds to Thursdays, and includes object 616b-1, which indicates that the proposed workout plan includes a 15-minute HIIT workout each Thursday, and object 616b-2, which indicates that the proposed workout plan includes 15-minute strength workout each Thursday. A user can select objects 616b-1, 616b-2, 616b-3 to open user interfaces similar to those shown in FIGS. 6E-6G above to modify workouts scheduled for Thursdays in the user-defined workout plan. At FIG. 6J, electronic device 600 detects user input 624 corresponding to selection of option 614c.

At FIG. 6K, in response to user input 624, electronic device 600 generates a workout plan that includes a plurality of scheduled workouts, and displays user interface 626 corresponding to the workout plan. User interface 626 includes plan name information 626a, and plan duration information 626b, which indicates that the workout plan will go for four weeks, and the current week is the first week of the workout plan. User interface 626 also includes weekly schedule indication 626c, which identifies which days in the next seven days have scheduled workouts (e.g., Tuesday, Thursday, and Saturday), and which days in the next seven days do not have scheduled workouts. In FIG. 6K, Tuesday the 15^{th} is selected in weekly schedule indication 626c (e.g., as indicated by the circle around Tuesday the 15^{th}), and user interface displays workouts scheduled for Tuesday the 15^{th}. User interface 626 includes workout representation 626d, which indicates that a 10-minute HIIT workout entitled “HIIT with Kim” is scheduled for Tuesday the 15^{th}, and workout representation 626f, which indicates that a 20-minute strength workout entitled “Strength with Gregg” is also scheduled for Tuesday the 15^{th}. In some embodiments, these workouts are automatically selected (e.g., by electronic device 600) based on the information selected and/or provided by the user in FIGS. 6B-6J. Workout representation 626d is displayed with a swap workout option 626e to swap out the “HIIT with Kim” workout scheduled for Tuesday, and workout representation 626f is displays with a swap workout option 626g that is selected to swap out the “Strength with Gregg” workout scheduled for Tuesday. At FIG. 6K, electronic device 600 detects user input 628 corresponding to selection of option 626c.

At FIG. 6L, in response to user input 628, electronic device 600 displays user interface 630. User interface 630 displays various workout options 630d, 630e that the user can select to replace the “HIIT with Kim” workout scheduled for Tuesday the 15^{th}. In some embodiments, the workout options displayed in user interface 630 are selected based on the workouts having the same workout type as the HIIT with Kim workout (e.g., HIIT), and having the same duration as the HIIT with Kim workout (e.g., 10 minutes). In the depicted embodiment, user interface 630 also includes duration options 630a, 630b, 630c that a user can select to view workout options having different durations, but the same workout type as the HIIT with Kim workout (e.g., option 630b that is selectable to view 20-min HIIT workouts, and option 630c that is selectable to view 30-min HIIT workouts). At FIG. 6L, electronic device 600 detects user input 631 corresponding to selection of close option 630f.

At FIG. 6M, in response to user input 631, electronic device ceases display of user interface 630, and displays user interface 626. At FIG. 6M, electronic device 600 detects user input 634 corresponding to selection of Thursday the 17^{th} within weekly schedule indication 626c.

At FIG. 6N, in response to user input 634, electronic device 600 ceases display of workout representations 626d, 626f in user interface 626 (e.g., workouts that are scheduled for Tuesday the 15^{th}), and displays workout representations 626i, 626k, which are representative of workouts scheduled for Thursday the 17^{th}. As shown in FIG. 6N, a 15-minute HIIT workout and a 15-minute strength workout are scheduled for Thursday the 17^{th}. As discussed above, workouts corresponding to workout representations 626i, 626k (e.g., HIIT with Bakari and Strength with Gregg) are automatically selected (e.g., by electronic device 600) based on information provided and/or selected by the user in the user interfaces shown in FIGS. 6B-6J. At FIG. 6N, electronic device 600 detects user input 636 corresponding to selection of option 626h.

At FIG. 6N, in response to user input 636, electronic device 600 displays workout selection user interface 604, which was described above. However, in FIG. 6O, based on the user creating a new workout plan, workout selection user interface 604 now displays workout representation 608d that is representative of a workout scheduled for the current day (e.g., a 10-minute HIIT with Kim workout). Workout representation 608d is displayed with swap workout option 608f and start workout option 608g that is selected to initiate the workout. In FIG. 6O, workout selection user interface 604 also includes workout representation 608h, which corresponds to a workout that is not a part of the user's workout plan. At FIG. 6O, electronic device 600 detects user input 638 corresponding to selection of workout representation 608h.

It can be seen in FIG. 6N that workout representation 608h corresponds to a workout that is of the same workout type as scheduled workout 608d (e.g., HIIT), and has a duration that is longer than scheduled workout 608d. At FIG. 6P, in response to user input 638, and based on a determination that the selected workout has the same workout type as today's scheduled workout and a duration that is at least as long as today's scheduled workout, electronic device 600 displays user interface 640 which asks the user if the user wishes to replace today's scheduled workout (HIIT with Kim) with the selected workout (HIIT with Bakari). User interface 640 includes option 640a that is selectable to replace today's scheduled workout with the selected workout and initiate the selected workout, and option 640b that is selectable to forgo replacing today's scheduled workout with the selected workout. In some embodiments, selecting option 640b results in initiating the selected workout (e.g., similar to what is shown in FIG. 6Q), but while still maintaining HIIT with Kim as the scheduled workout for today that the user must complete in order to meet plan requirements for the day. At FIG. 6P, electronic device 600 detects user input 642 corresponding to selection of option 640a.

At FIG. 6Q, in response to user input 642, electronic device initiates a workout session corresponding to the selected workout, including displaying user interface 644. User interface 644 includes video content 644a corresponding to the selected workout, workout metrics 644b which display workout metrics for the workout session, and daily activity metrics 644c.

At FIG. 6R, electronic device 600 displays user interface 626 corresponding to the user-defined workout plan. In FIG. 6R, user interface 626 is displaying workouts scheduled for today, Tuesday the 15^{th}. Previously, in FIG. 6M, user interface 626 displayed a 10-minute HIIT with Kim workout as being scheduled for Tuesday the 15^{th}. However, based on user selection of option 640a in FIG. 6P, user interface 626 now displays the 15-minute HIIT with Bakari workout as being the scheduled workout for today rather than the 10-minute HIIT with Kim workout.

FIG. 6S depicts a scenario in which it is the next day, and it is now Wednesday the 16^{th}, and electronic device 600 displays workout selection user interface 604. There is no scheduled workout for Wednesday the 16^{th} in the user-defined workout plan. Accordingly, user interface 604 displays workout representation 608i along with an indication that workout representation 608i is scheduled for Thursday. Workout representation 608i is presented with swap workout option 608j, start workout option 608k, and view plan option 608e. User interface 604 also includes workout representation 608l, which is representative of a workout that is not scheduled in and/or is not part of the user-defined workout plan. At FIG. 6S, electronic device 600 detects user input 645 corresponding to selection of option 608k.

At FIG. 6T, in response to user input 645, and based on a determination that workout 608l is scheduled for a future date according to the user-defined workout plan, electronic device 600 displays user interface 646, which indicates that the selected workout is scheduled for a future date in the workout plan. User interface 646 includes option 646a that is selectable to initiate a workout session corresponding to the selected workout, and option 646b that is selectable to forgo performing the workout. In some embodiments, performing the future-scheduled workout on a day in which no workouts are planned according to the workout plan does not count as completion of a workout plan for record-keeping purposes.

FIG. 6U depicts a scenario in which it is the next day, and it is now Thursday the 17^{th}, and electronic device 600 displays workout selection user interface 604. User interface 604 now indicates that the HIIT with Bakari workout represented by workout representation 608i is scheduled for today. At FIG. 6U, electronic device 600 detects user input 648 corresponding to selection of option 608k. At FIG. 6V1, in response to user input 648, electronic device 600 initiates a workout session corresponding to the selected workout, including displaying user interface 650, which includes video content 650a corresponding to the selected workout, workout session metrics 650b, and daily activity metrics 650c. It can be seen that selection of workout representation 608i did not result in display of user interface 646, because in FIG. 6U, the workout corresponding to workout representation 608i is scheduled for the current day (and not a future date).

FIG. 6V2 depicts electronic device 600, as well as electronic device 656, which is a smart watch with touch-sensitive display 658. In some embodiments, electronic device 656 and electronic device 600 correspond to the same user. For example, in some embodiments, electronic device 656 and electronic device 600 are logged into the same user account. At FIG. 6V2, based on a determination that the user has one or more workouts scheduled for the current day according to the user-defined workout plan, electronic device 600 displays notification 654, and electronic device 656 displays notification 654b.

FIG. 6W depicts an example scenario in which the user-defined workout plan has completed (e.g., the duration of the user-defined workout plan has elapsed). In response to a determination that the user-defined workout plan has completed, electronic device 600 displays workout plan summary user interface 660. User interface 660 includes total workout duration information 660a (e.g., the total duration of all plan workouts performed by the user during the duration of the user-defined workout plan), total calorie information 660b (e.g., the total number of calories burned by the user in plan workouts during the duration of the user-defined workout plan), indication 660c that indicates the total number of plan workouts the user performed during the duration of the user-defined workout plan, and the total duration 660d of the user-defined workout plan. User interface 660 also includes option 660e that is selectable to cease display of user interface 660, and option 660f that is selectable to indicate a user request to initiate a new workout plan with the same parameters as the completed workout plan. At FIG. 6W, electronic device 600 detects user input 662 corresponding to selection of option 660f.

At FIG. 6X, in response to user input 662, electronic device 600 displays user interface 664. User interface 664 displays plan parameters for the just-completed workout plan (and for the new workout plan the user has requested to initiate), including workout days 664a, daily workout duration 664b, workout types 664c, and plan duration 664d. User interface 664 includes option 664e that is selectable to initiate a new workout plan using the workout parameters shown in user interface 664.

FIGS. 6Y-6GG illustrate example user interfaces pertaining to a user-defined workout plan, but on a different type of device. FIG. 6Y depicts electronic device 668, which is an entertainment center that includes television 669. In some embodiments, electronic device 668 corresponds to the same user as electronic device 600. For example, in some embodiments, electronic device 668 is logged into the same user account as electronic device 600. At FIG. 6Y, electronic device 668 displays workout selection user interface 670, which includes workout representations 670a-670d that are selectable by a user to initiate a workout session and display corresponding workout content. In FIG. 6Y, there is no user-defined workout plan that is active for the user. Accordingly, workout representations 670a-670d are not selected based on any workout plan parameters.

At FIG. 6Z, the user has initiated a user-defined workout plan (e.g., using electronic device 600). In some embodiments, a user is not able to define and/or generate a workout plan on electronic device 668. In some embodiments, the user must use a different type of device (e.g., electronic device 600) to create a user-defined workout plan. At FIG. 6Z, the user has used electronic device 600 to initiate a user-defined workout plan. At FIG. 6Z, based on a determination that the user has initiated a user-defined workout plan on another device, user interface 670 now displays workout representation 670e corresponding to the user-defined workout plan. Workout representation 670e is displayed with option 670e-1 that is selectable to initiate a workout session corresponding to the selected workout, and option 670e-2 that is selectable to view workout plan information. At FIG. 6Z, electronic device 668 detects user input 672 (e.g., a remote control input) corresponding to selection of option 670c-1.

At FIG. 6AA, in response to user input 672, electronic device 688 initiate a workout session corresponding to the selected workout, and displays user interface 674. User interface 674 includes video content 674a corresponding to the selected workout, workout metrics 674b, and daily physical activity metrics 674c.

In FIG. 6BB, it is the next day, and there are not scheduled workouts for the current day according to the user-defined workout plan. Accordingly, user interface 670 displays workout representation 670f that corresponds to a workout that is scheduled for a future day. User interface 670 also displays workout representations 670b-670d that correspond to non-plan workouts. Workout representation 670f is presented with options 670f-1, 670f-2. At FIG. 6BB, electronic device 688 detects user input 676 (e.g., a remote control input) corresponding to selection of option 670f-2.

At FIG. 6CC, in response to user input 676, electronic device 668 displays user interface 678 which displays information about the active user-defined workout plan. User interface 678 displays workout representation 678a, 678b that correspond to workouts scheduled for the next scheduled workout day. User interface 678 also includes option 678c. At FIG. 6CC, electronic device 668 detects user input 679 (e.g., a remote control input) corresponding to selection of option 678c.

At FIG. 6DD, in response to user input 679, electronic device 600 displays options 678c-1, 678c-2, which are selectable to edit the name of the workout plan and to prematurely end the workout plan, respectively. At FIG. 6DD, electronic device 668 detects user input 680 (e.g., a remote control input (e.g., a press and hold input)).

At FIG. 6EE, in response to user input 680, in response to user input 680, electronic device 668 displays options 682a-682f. Option 682a is selectable to swap the workout HIIT with Bakari with another workout that matches plan parameters. Option 682b is selectable to initiate a workout session corresponding to the selected workout. Option 682c is selectable to save the workout to a library corresponding to the user. Option 682d is selectable to save the workout to a collection of workouts, as will be described in greater detail below with reference to FIGS. 8A-8V. Option 682e is selectable to view the collection of workouts, as will be described in greater detail below with reference to FIGS. 8A-8V. Option 628f is selectable to view more information about the selected workout. At FIG. 6EE, electronic device 668 detects user input 684b (e.g., an up navigation input) and user input 684a (e.g., a down navigation input).

At FIG. 6FF, in response to user input 684a, electronic device 668 displays scrolling of user interface 678 to reveal workout representations 678c-678e corresponding to past workouts that were scheduled in the workout plan. In some embodiments, workout representations 678c-678e are selectable to initiate a workout session corresponding to the selected workout. At FIG. 6GG, in response to user input 684b, electronic device 668 displays workout representations 678f-678k corresponding to future scheduled workouts according to the workout plan. In some embodiments, workout representations 678f-678k are selectable to initiate a process for initiating a workout session corresponding to the selected workout.

FIG. 7 is a flow diagram illustrating a method for providing and accessing workout content using a computer system in accordance with some embodiments. Method 700 is performed at a computer system (e.g., 100, 300, 500, 600, 656, and/or 668) that is in communication with one or more display generation components (e.g., a display, a touch-sensitive display, a television, a monitor, a visual output device, a 3D display, a display having at least a portion that is transparent or translucent on which images can be projected (e.g., a see-through display), a projector, a heads-up display, and/or a display controller) and one or more input devices (e.g., a touch-sensitive surface (e.g., a touch-sensitive display); a mouse; a keyboard; a remote control; a visual input device (e.g., one or more cameras (e.g., an infrared camera, a depth camera, a visible light camera, and/or a gaze tracking camera)); an audio input device; a biometric sensor (e.g., a fingerprint sensor, a face identification sensor, a gaze tracking sensor, and/or an iris identification sensor) and/or one or more mechanical input devices (e.g., a depressible input mechanism; a button; a rotatable input mechanism; a crown; and/or a dial)). Some operations in method 700 are, optionally, combined, the orders of some operations are, optionally, changed, and some operations are, optionally, omitted.

In some embodiments, the electronic device (e.g., 600, 656, 668) is a computer system. The computer system is optionally in communication (e.g., wired communication, wireless communication) with a display generation component and with one or more input devices. The display generation component is configured to provide visual output, such as display via a CRT display, display via an LED display, or display via image projection. In some embodiments, the display generation component is integrated with the computer system. In some embodiments, the display generation component is separate from the computer system. The one or more input devices are configured to receive input, such as a touch-sensitive surface receiving user input. In some embodiments, the one or more input devices are integrated with the computer system. In some embodiments, the one or more input devices are separate from the computer system. Thus, the computer system can transmit, via a wired or wireless connection, data (e.g., image data or video data) to an integrated or external display generation component to visually produce the content (e.g., using a display device) and can receive, a wired or wireless connection, input from the one or more input devices.

As described below, method 700 provides an intuitive way for providing and accessing workout content. The method reduces the cognitive burden on a user for providing and accessing workout content, thereby creating a more efficient human-machine interface. For battery-operated computing devices, enabling a user to access workout content faster and more efficiently conserves power and increases the time between battery charges.

The computer system receives (702), via the one or more input devices, a first set of user inputs (e.g., a first set of user inputs and/or one or more user inputs) (e.g., one or more touch inputs, one or more non-touch inputs, and/or one or more gestures) (in some embodiments, a first set of user inputs corresponding to a user request to generate a workout plan (e.g., a weekly workout plan and/or a monthly workout plan)), including: one or more user inputs (e.g., 611a, 611b, 611c) (e.g., one or more touch inputs, one or more non-touch inputs, and/or one or more gestures) corresponding to user selection of one or more workout days (e.g., 610a) (e.g., one or more days of the week (e.g., Monday, Tuesday, Wednesday, Thursday, Friday, Saturday, and/or Sunday) and/or one or more days in a calendar month (e.g., the first of the month, the second of the month, the third of the month, and so forth)); and one or more user inputs (e.g., 611d, 611c) (e.g., one or more touch inputs, one or more non-touch inputs, and/or one or more gestures) corresponding to user selection of one or more workout types (e.g., 610c-610k) (e.g., one or more workout modalities (e.g., running, cycling, swimming, HIIT, yoga, core, rowing, strength, and/or dance), one or more muscle groups and/or body parts (e.g., legs, arms, shoulders, chest, and/or back), indoor workout, outdoor workout, and/or track workout).

In response to receiving the first set of user inputs, the computer system (e.g., 600) displays (704), via the one or more display generation components, a workout plan user interface (e.g., 614 and/or 626), including: a representation of a first day (e.g., 616a) that is selected (e.g., from a plurality of days and/or a plurality of possible days) based on the one or more user inputs corresponding to user selection of one or more workout days; a representation of a second day (e.g., 616b) different from the first day that is selected (e.g., from a plurality of days and/or a plurality of possible days) based on the one or more user inputs corresponding to user selection of one or more workout days; an indication of a first workout (e.g., 616a-1, 616a-2) (e.g., a representation of the first workout, an object corresponding to the first workout, and/or text corresponding to the first workout) (e.g., a first workout that corresponds to a first workout type of a plurality of workout types and/or a first workout duration) corresponding to the first day (e.g., a first workout scheduled to be performed on the first day) and that is selected (e.g., from a plurality of workouts and/or from a plurality of workout options) based on the one or more user inputs corresponding to user selection of one or more workout types; and an indication of a second workout (e.g., 616a-2, 616b-1, 616b-2) (e.g., a representation of the second workout, an object corresponding to the second workout, and/or text corresponding to the second workout) (e.g., a second workout that corresponds to a second workout type of a plurality of workout types and/or a second workout duration) corresponding to the second day (e.g., a second workout scheduled to be performed on the second day) and that is selected (e.g., from a plurality of workouts and/or from a plurality of workout options) based on the one or more user inputs corresponding to user selection of one or more workout types. Enabling a user to provide user inputs selecting one or more workout days and one or more workout types to create a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently. Furthermore, displaying the workout plan user interface provides the user with feedback about a state of the device.

In some embodiments, the one or more user inputs (e.g., 611a, 611b, 611c) corresponding to user selection of one or more workout days specifies a number of workout days per week (e.g., the user selection of one or more workout days specifies how many days per week the user intends to perform a workout). Enabling a user to provide user inputs selecting one or more workout days to create a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the one or more user inputs (e.g., 611a, 611b, 611c) corresponding to user selection of one or more workout days specifies one or more days of the week (e.g., Monday, Tuesday, Wednesday, Thursday, Friday, Saturday, and/or Sunday). Enabling a user to provide user inputs selecting one or more workout days to create a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the first set of user inputs further include: one or more user inputs (e.g., one or more touch inputs, one or more non-touch inputs, and/or one or more gestures) corresponding to identification (e.g., selection and/or entry) of one or more workout durations (e.g., 610b) (e.g., time durations) (e.g., workout durations for one or more workout days (e.g., a first number of minutes per day; and/or a first number of minutes for a first day and a second number of minutes for a second day)). Enabling a user to provide user inputs to specify one or more workout durations for a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the one or more user inputs corresponding to identification of one or more workout durations comprises identification of a single workout duration value (e.g., 610b) (e.g., not more than one workout duration value) (e.g., specifying that the user would like to work out a first number of minutes and/or hours on each workout day). Enabling a user to provide user inputs to specify one or more workout durations for a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the one or more user inputs corresponding to identification of one or more workout durations comprises identification of a plurality of workout duration values (e.g., separate workout durations selected in user interfaces 618a, 618b for different days of the week) (e.g., specifying a first workout duration for a first workout, a first workout modality, a first type of workout, and/or a first workout day; and a second workout duration for a second workout, a second workout modality, a second type of workout, and/or a second workout day). Enabling a user to provide user inputs to specify one or more workout durations for a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the plurality of workout duration values includes: a first workout duration value (e.g., a first number of minutes and/or hours) corresponding to a first day (e.g., in FIG. 6E, selecting a workout duration 618a-2 through 618a-5 for a first day of the week) of the one or more workout days (e.g., a first date and/or a first day of the week); and a second workout duration value (e.g., the same as the first workout duration value or different from the first workout duration value) (e.g., a second number of minutes and/or hours) corresponding to a second day (e.g., in FIG. 6F, selecting a workout duration 618b-2 through 618b-5 for a second day) of the one or more workout days different from the first day (e.g., a second date and/or a second day of the week). Enabling a user to provide user inputs to specify one or more workout durations for a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the first set of user inputs further comprises: one or more user inputs (e.g., one or more touch inputs, one or more non-touch inputs, and/or one or more gestures) corresponding to user selection of one or more trainers (e.g., FIG. 6H) (e.g., people, personal trainers, and/or people leading workouts) of a plurality of trainers (e.g., user identification of one or more trainers that the user prefers). Enabling a user to provide user inputs to specify one or more trainers for a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the computer system displays, via the one or more display generation components, representations of a first subset (e.g., that includes some or all of the plurality of trainers) of the plurality of trainers, wherein the first subset is selected from the plurality of trainers based on the one or more user inputs corresponding to user selection of one or more workout types (e.g., the first subset of trainers is selected based on the first subset of trainers being associated with and/or corresponding to one or more workout types selected by the user). Enabling a user to provide user inputs to specify one or more trainers for a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the one or more user inputs corresponding to user selection of one or more trainers defines a first set of user-selected trainers; the first workout corresponds to a first trainer of the first set of user-selected trainers (e.g., the first workout is selected based on the first trainer being in the first set of user-selected trainers); and the second workout corresponds to a second trainer of the plurality of trainers that is not in the first set of user-selected trainers (e.g., is a trainer that was not selected and/or identified by the user). In some embodiments, the one or more trainers selected by the user are given greater weight and/or more heavily weighted and/or preferred than non-selected trainers, but workouts by non-selected trainers are not excluded from selection. Enabling a user to provide user inputs to specify one or more trainers for a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the first set of user inputs further comprises: one or more user inputs (e.g., one or more touch inputs, one or more non-touch inputs, and/or one or more gestures) corresponding to user selection of one or more music types (e.g., FIG. 6I) (e.g., types of music and/or music genres) of a plurality of types (e.g., user identification of one or more types of music (e.g., workout music) that the user prefers). Enabling a user to provide user inputs to specify one or more music types for a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the first set of user inputs further comprises: one or more user inputs (e.g., one or more touch inputs, one or more non-touch inputs, and/or one or more gestures) defining a first workout plan duration (e.g., 614a) (e.g., a duration for a workout plan (e.g., 1 week, 2 weeks, 3 weeks, 4 weeks, 5 weeks, 6 weeks, 7 weeks, 8 weeks, one month, and/or two months) and/or a duration for which the computer system will select and/or schedule workouts). Enabling a user to provide user inputs to specify a duration for a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the first set of user inputs correspond to a user request to generate a workout plan (e.g., user input 624) (e.g., a workout plan having a finite duration, a workout schedule, and/or a schedule of planned workouts); the workout plan user interface (e.g., 626) corresponds to the workout plan (e.g., displays one or more workouts selected (e.g., automatically selected) for the workout plan). Subsequent to displaying the workout plan user interface, the computer system displays, via the one or more display generation components, a workout selection user interface that comprises a plurality of workout platters corresponding to a plurality of workouts, including: displaying, in a first region of the workout selection user interface, a first workout platter (e.g., user interface object and/or affordance) associated with the first workout (e.g., in some embodiments, a first workout platter that is selectable to initiate the first workout and/or a initiate a workout session corresponding to the first workout; in some embodiments, the first workout platter is selectable to cause display of audio-visual content corresponding to the first workout (e.g., video content that depicts an instructor and/or trainer performing one or more actions and/or activities to be emulated and/or performed by a user of the computer system)) (in some embodiments, the first workout platter is selected for display in the first region of the workout selection user interface based on the first workout being part of the workout plan); and displaying, in a second region of the workout selection user interface, a second workout platter (e.g., user interface object and/or affordance) associated with a third workout (e.g., in some embodiments, a second workout platter that is selectable to initiate the third workout and/or a initiate a workout session corresponding to the third workout; in some embodiments, the second workout platter is selectable to cause display of audio-visual content corresponding to the third workout (e.g., video content that depicts an instructor and/or trainer performing one or more actions and/or activities to be emulated and/or performed by a user of the computer system)) different from the first workout, wherein the third workout is not part of the workout plan (e.g., is not scheduled in the workout plan). Automatically selecting workouts for a user based on a workout plan allows for this operation to be performed with additional user input.

In some embodiments, the computer system displays via the one or more display generation components, representations of a plurality of workout types (e.g., 610c-610k) (e.g., a plurality of workout modalities (e.g., running, cycling, swimming, HIIT, yoga, core, rowing, strength, and/or dance), a plurality of muscle groups and/or body parts (e.g., legs, arms, shoulders, chest, and/or back), indoor workout, outdoor workout, and/or track workout), wherein: the one or more user inputs corresponding to user selection of the one or more workout types comprises one or more user inputs interacting with the representations of the plurality of workout types (e.g., selecting one or more of the representations of the plurality of workouts); and the plurality of workout types are selected for display based on previous workouts completed by a user of the computer system (e.g., the plurality of workout types are selected form a larger set of workout types based on previous workouts completed by a user of the computer system). In some embodiments, the computer system displays representations of a plurality of trainers and/or music types for selection by a user (e.g., to be used in a user-defined workout plan), and the representations of the plurality of trainers and/or music types are determined based on previous workouts completed by the user of the computer system. Enabling a user to provide user inputs to specify one or more trainers for a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the first set of user inputs correspond to a user request to generate a first workout plan (e.g., user input 624) (e.g., a workout plan having a finite duration, a workout schedule, and/or a schedule of planned workouts); the workout plan user interface corresponds to the first workout plan (e.g., displays one or more workouts selected (e.g., automatically selected) for the workout plan). Subsequent to displaying the workout plan user interface, the computer system displays, via the one or more display generation components, a first workout selection user interface (e.g., 604) that comprises a first plurality of workout platters (e.g., 608d, 608h) corresponding to a first plurality of workouts, including: displaying, in a first display region of the first workout selection user interface, a first platter (e.g., 608d) (e.g., user interface object and/or affordance) associated with the first workout (e.g., in some embodiments, a first platter that is selectable to initiate the first workout and/or a initiate a workout session corresponding to the first workout; in some embodiments, the first platter is selectable to cause display of audio-visual content corresponding to the first workout (e.g., video content that depicts an instructor and/or trainer performing one or more actions and/or activities to be emulated and/or performed by a user of the computer system)), wherein the first workout platter is selected for display in the first region of the workout selection user interface based on the first workout being part of the first workout plan (e.g., based on the first workout being a next scheduled workout in the first workout plan and/or based on the first workout being scheduled for a current day according to the first workout plan). In some embodiments, displaying the first workout selection user interface further comprises, displaying, in a second display region of the first workout selection user interface, a second platter associated with a first non-plan workout different from the first workout, wherein the first non-plan workout is not part of the workout plan. Automatically selecting workouts for a user based on a workout plan allows for this operation to be performed with additional user input.

Note that details of the processes described above with respect to method 700 (e.g., FIG. 7) are also applicable in an analogous manner to the methods described below. For example, method 700 optionally includes one or more of the characteristics of the various methods described below with reference to methods 800, 900, and/or 1100. For example, in some embodiments, the workout plan recited in method 800 is the workout plan recited in method 700 and/or method 900. For brevity, these details are not repeated below.

FIG. 8 is a flow diagram illustrating a method for providing and accessing workout content using a computer system in accordance with some embodiments. Method 700 is performed at a computer system (e.g., 100, 300, 500) that is in communication with one or more display generation components (e.g., a display, a touch-sensitive display, a television, a monitor, a visual output device, a 3D display, a display having at least a portion that is transparent or translucent on which images can be projected (e.g., a see-through display), a projector, a heads-up display, and/or a display controller) and one or more input devices (e.g., a touch-sensitive surface (e.g., a touch-sensitive display); a mouse; a keyboard; a remote control; a visual input device (e.g., one or more cameras (e.g., an infrared camera, a depth camera, a visible light camera, and/or a gaze tracking camera)); an audio input device; a biometric sensor (e.g., a fingerprint sensor, a face identification sensor, a gaze tracking sensor, and/or an iris identification sensor) and/or one or more mechanical input devices (e.g., a depressible input mechanism; a button; a rotatable input mechanism; a crown; and/or a dial)). Some operations in method 700 are, optionally, combined, the orders of some operations are, optionally, changed, and some operations are, optionally, omitted.

As described below, method 700 provides an intuitive way for providing and accessing workout content. The method reduces the cognitive burden on a user for providing and accessing workout content, thereby creating a more efficient human-machine interface. For battery-operated computing devices, enabling a user to access workout content faster and more efficiently conserves power and increases the time between battery charges.

The computer system (e.g., 600) receives, via the one or more input devices, a first set of user inputs (e.g., a first set of user inputs and/or one or more user inputs) (e.g., one or more touch inputs, one or more non-touch inputs, and/or one or more gestures).

In response to receiving the first set of user inputs, the computer system displays (804), via the one or more display generation components, a workout selection user interface (e.g., 604), including: displaying (806), in a first region of the workout selection user interface, a first workout platter (e.g., user interface object and/or affordance) associated with a first respective workout (e.g., in some embodiments, a first workout platter that is selectable to initiate a first respective workout and/or a initiate a workout session corresponding to the first respective workout; in some embodiments, the first workout platter is selectable to cause display of audio-visual content corresponding to the first respective workout (e.g., video content that depicts an instructor performing one or more actions and/or activities to be emulated and/or performed by a user of the computer system)); and displaying (808), in a second region of the workout selection user interface different from the first region, a second workout platter (e.g., user interface object and/or affordance) different from the first workout platter and associated with a second respective workout (e.g., in some embodiments, a second workout platter that is selectable to initiate a second respective workout and/or a initiate a workout session corresponding to the second respective workout; in some embodiments, the second workout platter is selectable to cause display of audio-visual content corresponding to the second respective workout (e.g., video content that depicts an instructor performing one or more actions and/or activities to be emulated and/or performed by a user of the computer system)) different from the first respective workout, wherein: in accordance with a determination that a first set of criteria is satisfied (810), including a first criterion that is satisfied when a user-defined workout plan is active (e.g., for a user of the computer system) (e.g., when the one or more user inputs are received) (e.g., a user-defined workout plan that is associated with one or more user-selected workout days (e.g., one or more days of the week (e.g., Monday, Tuesday, Wednesday, Thursday, Friday, Saturday, and/or Sunday) and/or one or more days in a calendar month (e.g., the first of the month, the second of the month, the third of the month, and so forth)), and/or one or more user-selected workout types (e.g., one or more workout modalities (e.g., running, cycling, swimming, HIIT, yoga, core, rowing, strength, and/or dance), one or more muscle groups and/or body parts (e.g., legs, arms, shoulders, chest, and/or back), indoor workout, outdoor workout, and/or track workout)), the first workout platter corresponds to a first workout that is selected based on the user-defined workout plan (e.g., 608d in FIG. 6O) (e.g., a first workout that is selected based on one or more user-selected workout days, a first workout that is selected based on one or more user-selected workout durations, and/or a first workout that is selected based on one or more user-selected workout types); and in accordance with a determination that a user-defined workout plan is not active (812) (e.g., when the one or more user inputs are received) (e.g., the first set of criteria is not satisfied and/or the first criterion is not satisfied), the first workout platter corresponds to a second workout different from the first workout (e.g., 608a in FIG. 6A). Automatically selecting workouts for a user based on a workout plan allows for this operation to be performed with additional user input. Furthermore, displaying different workout in the workout selection user interface based on whether or not a user-defined workout plan is active enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while the user-defined workout plan is active, wherein the user-defined workout plan includes a first user-specified set of workout days (e.g., a set of days on which workouts are scheduled (e.g., one or more days of the week (e.g., Monday, Tuesday, Wednesday, Thursday, Friday, Saturday, and/or Sunday))), the computer system displays, via the one or more display generation components, the workout selection user interface (e.g., 604), wherein: in accordance with a determination that a current day is in the first user-specified set of workout days (e.g., today is a scheduled workout day according to the first user-defined workout plan), the first workout platter corresponds to a third workout that is scheduled for the current day according to the user-defined workout plan (e.g., 608i in FIG. 6U); and in accordance with a determination that the current day is not in the first user-specified set of workout days (e.g., today is not a scheduled workout day according to the first user-defined workout plan), the first workout platter corresponds to a fourth workout different from the third workout (e.g., in some embodiments, in FIG. 6S, rather than displaying workout 608i, user interface 604 includes a different non-plan workout) (e.g., a fourth workout that is selected based on the user-defined workout plan (e.g., a future scheduled workout according to the user-defined workout plan) and/or a fourth workout that is not selected based on the user-defined workout plan). Automatically selecting workouts for a user based on a workout plan allows for this operation to be performed with additional user input. Furthermore, displaying different workout in the workout selection user interface based on whether or not the current day is in the user-specified set of workout days enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, displaying the workout selection user interface (e.g., 604) while the first user-defined workout plan is active further comprises: in accordance with a determination that the current day is not in the first user-specified set of workout days, displaying an indication that the current day is not in the first user-specified set of workout days (e.g., FIG. 6S, “For Thursday”). Displaying an indication that the current day is not in the first user-specified set of workout days enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the user-defined workout plan includes: a first user-specified workout duration (e.g., in minutes and/or hours) for a first user-specified workout day (e.g., a calendar date and/or a day of the week); and a second user-specified workout duration for a second user-specified workout day, wherein the second user-specified workout duration is different from the first user-specified workout duration, and the second user-specified workout day is different from the first user-specified workout day. In some embodiments, while the user-defined workout plan is active, the computer system displays, via the one or more display generation components, the workout selection user interface (e.g., 604), wherein: in accordance with a determination that a current day corresponds to the first user-specified workout day, the first workout platter corresponds to a fifth workout that is selected based on the fifth workout having a workout duration that corresponds to the first user-specified workout duration (e.g., is the same as the first user-specified workout duration); and in accordance with a determination that the current day corresponds to the second user-specified workout day, the first workout platter corresponds to a sixth workout different from the fifth workout and that is selected based on the sixth workout having a workout duration that corresponds to the second user-specified workout duration (e.g., is the same as the second user-specified workout duration). Automatically selecting workouts for a user based on a workout plan allows for this operation to be performed with additional user input. Furthermore, displaying different workout in the workout selection user interface based on whether or not a user-defined workout plan is active enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while the user-defined workout plan is active, the computer system displays, via the one or more display generation components, a first notification (e.g., 654, 654b) (e.g., a push notification and/or a banner notification) indicative of a workout being scheduled for the current day (e.g., that identifies the workout scheduled for the current day and/or indicating that a workout is scheduled for the current day). Displaying a notification indicative of a workout being scheduled for the current day enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, while displaying the first notification (e.g., 654, 654b), the computer system receives, via the one or more input devices, a selection input (e.g., one or more touch inputs, one or more non-touch inputs, and/or one or more gestures) corresponding to selection of the first notification; and in response to receiving the selection input corresponding to selection of the first notification, the computer system displays, via the one or more display generation components, a first user interface (e.g., 626) corresponding to the workout scheduled for the current day (e.g., a first user interface that includes a first selectable object that is selectable to initiate the workout scheduled for the current day, and/or a first user interface that identifies one or more of: a trainer, a workout duration, and/or a workout type (e.g., workout modality) of the workout scheduled for the current day). Displaying a notification indicative of a workout being scheduled for the current day and that is selectable to display a user interface corresponding to the workout enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, the first notification (e.g., 654) is displayed as part of a lock screen user interface (e.g., 652) indicative of the computer system being in a lock state (e.g., a state in which a user is prevent from accessing one or more features of the computer system, and/or in which user authentication information is required in order to transition the computer system into an unlocked state that includes access to the one or more features of the computer system). Displaying a notification indicative of a workout being scheduled for the current day enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, a second notification (e.g., 654b) corresponding to the first notification (e.g., 654) (e.g., a second notification indicative of a workout being scheduled for the current day) is displayed on a first external device (e.g., 656) separate from the computer system. In some embodiments, the first external device corresponds to the computer system (e.g., is associated with the same user as the computer system, and/or is logged into the same user account as the computer system). Displaying, on multiple devices, a notification indicative of a workout being scheduled for the current day enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, while the user-defined workout plan is active, the computer system displays, via the one or more display generation components, a scheduled workout user interface (e.g., 604) that includes: a start workout option (e.g., 608g) that is selectable to initiate a workout session corresponding to a first scheduled workout that is scheduled for the current day according to the user-defined workout plan; and a swap workout option (e.g., 608f) that is selectable to initiate a process for switching the first scheduled workout with a different workout. In some embodiments, initiating the workout session corresponding to the first scheduled workout comprises initiating recording of one or more physical activity metrics (e.g., heartrate, workout time elapsed, distance traversed, and/or calories burned) for the workout session (e.g., via one or more sensors in communication with the computer system and/or one or more sensors in a first external device that is in communication with the computer system)) (in some embodiments, initiating the workout session includes recording one or more physical activity metrics at a greater frequency than prior to initiation of the workout session). In some embodiments, initiating the workout session corresponding to the first scheduled workout comprises displaying video content corresponding to the first scheduled workout (e.g., video content that depicts an instructor performing one or more actions and/or activities to be emulated and/or performed by a user of the computer system). Providing users with selectable options to initiate a workout session and to switch out a workout in a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while displaying the scheduled workout user interface, the computer system receives, via the one or more input devices, a selection input corresponding to selection of the swap workout option (e.g., 608f); and in response to receiving the selection input corresponding to selection of the swap workout option: displays, via the one or more display generation components, one or more workout options (e.g., 630, 630d, 630e) corresponding to one or more workouts that are selected based on the one or more workouts having a same workout type as the first scheduled workout (e.g., the same workout modality) and a same workout duration as the first scheduled workout. Providing users with selectable options to switch out a workout in a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while displaying the scheduled workout user interface, the computer system receives, via the one or more input devices, a selection input corresponding to selection of the swap workout option (e.g., 608f); and in response to receiving the selection input corresponding to selection of the swap workout option: displays, via the one or more display generation components, one or more workout options (e.g., 630, 630d, 630e) corresponding to one or more workouts that are selected based on the one or more workouts having a same workout type as the first scheduled workout (e.g., the same workout modality), wherein at least some of the one or more workouts have a different workout duration from the first scheduled workout. Providing users with selectable options to switch out a workout in a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while the user-defined workout plan is active, the computer system detects that a user has completed a first respective workout; and in response to detecting that the user has completed the first respective workout: in accordance with a determination that the first respective workout is a scheduled workout according to the user-defined workout plan, displays, via the one or more display generation components, a first indication indicating that the user has completed a scheduled workout according to the user-defined workout plan; and in accordance with a determination that the first respective workout is not a scheduled workout according to the user-defined workout plan, forgoes display of the first indication. Displaying different content based on whether or not a completed workout is a workout from a user-defined workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, while the user-defined workout plan is active, the computer system detects that a user has completed a second respective workout; in response to detecting that the user has completed the second respective workout: in accordance with a determination that the second respective workout satisfies workout type criteria (e.g., the second respective workout has a workout type that matches a workout type scheduled for that day and/or for the current day) but the second respective workout does not satisfy workout duration criteria (e.g., the second respective workout has a workout duration that is less than a workout duration scheduled for that day and/or for the current day), forgoes display of the first indication indicating that the user has completed a scheduled workout according to the user-defined workout plan. Subsequent to detecting that the user has completed the second respective workout, the computer system detects that the user has completed a third respective workout (e.g., on the same day (e.g., the same calendar day and/or the same 24-hour period) as the second respective workout); and in response to detecting that the user has completed the third respective workout: in accordance with a determination that the third respective workout satisfies the workout type criteria, and the second respective workout and the third respective workout cumulatively satisfy the workout duration criteria (e.g., the second respective workout and the third respective workout cumulatively have a duration that is greater than or equal to the workout duration scheduled for the day and/or for the current day), displays the first indication that the user has completed a scheduled workout according to the user-defined workout plan. Displaying different content based on whether or not workout type criteria and workout duration criteria are met enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, at the end of the user-defined workout plan (e.g., after the user-defined workout plan has ended, and/or after a plan duration has expired), the computer system displays, via the one or more display generation components, a workout plan summary user interface (e.g., 660) that includes an indication of a number of workouts that the user completed according to the user-defined workout plan (e.g., 660c) (e.g., that matches workout plan parameters (e.g., workout type parameters and/or workout duration parameters)) (e.g., while the user-defined workout plan was active). Displaying the workout plan summary user interface at the end of the user-defined workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, the number of workouts that the user completed according to the user-defined workout plan (e.g., 660c) excludes one or more workouts that the user completed (e.g., while the user-defined workout plan was active) that do not match one or more criteria corresponding to the user-defined workout plan (e.g., do not match workout type criteria and/or workout duration criteria) (e.g., excludes one or more workouts that were of a workout type that is not a workout type that was selected for the user-defined workout plan; and/or excludes one or more workouts that did not satisfy a workout duration threshold according to the user-defined workout plan). Automatically excluding workouts that do not meet the one or more criteria from inclusion in the workout plan summary user interface allows for this operation to be performed automatically without further user input.

In some embodiments, the workout plan summary user interface includes a repeat option (e.g., 660f) that is selectable to repeat the user-defined workout plan (e.g., re-perform and/or restart the user-defined workout plan). Providing an option to repeat the user-defined workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the repeat option (e.g., 660f) is selectable to repeat the user-defined workout plan with the same workouts as were scheduled in the user-defined workout plan (e.g., without changing the workouts and/or repeating the same workouts that were used in the user-defined workout plan). Providing an option to repeat the user-defined workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the repeat option (e.g., 660f) is selectable to repeat the user-defined workout plan with different workouts (e.g., different workouts that satisfy the parameters of the user-defined workout plan (e.g., that satisfy workout type parameters, workout duration parameters, trainer, parameters, and/or music parameters)). Providing an option to repeat the user-defined workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while the user-defined workout plan is active, and a first scheduled workout is scheduled for a current day according to the user-defined workout plan, the computer system receives, via the one or more input devices, one or more user inputs corresponding to a user request to initiate a first non-scheduled workout that is different from the first scheduled workout (e.g., 638 in FIG. 6O) (e.g., and that is not scheduled for the current day, according to the user-defined workout plan); and in response to receiving the one or more inputs corresponding to the user request to initiate the first non-scheduled workout: in accordance with a determination that the first non-scheduled workout satisfies a first set of criteria with respect to the user-defined workout plan (e.g., the first non-scheduled workout is the same workout type as the first scheduled workout; and/or the first non-scheduled workout has the same workout duration as or a longer workout duration than the first scheduled workout), the computer system displays, via the one or more display generation components, a swap option (e.g., 640, 640a) that is selectable to replace the first scheduled workout with the first non-scheduled workout in the user-defined workout plan. Automatically displaying a swap option when a selected workout satisfies the first set of criteria enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, while the user-defined workout plan is active, the computer system receives, via the one or more input devices, one or more user inputs corresponding to a user request to initiate a first future scheduled workout that is scheduled for a future date according to the user-defined workout plan (e.g., 654 in FIG. 6S) (e.g., and is not scheduled for the current day, according to the user-defined workout plan); and in response to receiving the one or more user inputs corresponding to the user request to initiate the first future scheduled workout, displays, via the one or more display generation components, an indication (e.g., 646) that the first future scheduled workout is scheduled for a future date according to the user-defined workout plan. Displaying an indication that a selected workout is scheduled for a future date enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

Note that details of the processes described above with respect to method 800 (e.g., FIG. 8) are also applicable in an analogous manner to the methods described below and/or above. For example, method 800 optionally includes one or more of the characteristics of the various methods described herein with reference to method 700, 900, and/or 1100. For example, in some embodiments, the workout plan recited in method 800 is the workout plan recited in method 700 and/or method 900. For brevity, these details are not repeated below.

FIG. 9 is a flow diagram illustrating a method for providing and accessing workout content using a computer system in accordance with some embodiments. Method 900 is performed at a computer system (e.g., 100, 300, 500) that is in communication with one or more display generation components (e.g., a display, a touch-sensitive display, a television, a monitor, a visual output device, a 3D display, a display having at least a portion that is transparent or translucent on which images can be projected (e.g., a see-through display), a projector, a heads-up display, and/or a display controller) and one or more input devices (e.g., a touch-sensitive surface (e.g., a touch-sensitive display); a mouse; a keyboard; a remote control; a visual input device (e.g., one or more cameras (e.g., an infrared camera, a depth camera, a visible light camera, and/or a gaze tracking camera)); an audio input device; a biometric sensor (e.g., a fingerprint sensor, a face identification sensor, a gaze tracking sensor, and/or an iris identification sensor) and/or one or more mechanical input devices (e.g., a depressible input mechanism; a button; a rotatable input mechanism; a crown; and/or a dial)). Some operations in method 700 are, optionally, combined, the orders of some operations are, optionally, changed, and some operations are, optionally, omitted.

As described below, method 700 provides an intuitive way for providing and accessing workout content. The method reduces the cognitive burden on a user for providing and accessing workout content, thereby creating a more efficient human-machine interface. For battery-operated computing devices, enabling a user to access workout content faster and more efficiently conserves power and increases the time between battery charges.

The computer system (e.g., 668) receives (902), via the one or more input devices, a first set of user inputs (e.g., a first set of user inputs and/or one or more user inputs) (e.g., one or more touch inputs, one or more non-touch inputs, and/or one or more gestures).

In response to receiving the first set of user inputs, the computer system displays (904), via the one or more display generation components, a workout selection user interface (e.g., 670), including: displaying (906), in a first region of the workout selection user interface, a first workout platter (e.g., 670a,670c) (e.g., user interface object and/or affordance) associated with a first respective workout (e.g., in some embodiments, a first workout platter that is selectable to initiate a first respective workout and/or a initiate a workout session corresponding to the first respective workout; in some embodiments, the first workout platter is selectable to cause display of audio-visual content corresponding to the first respective workout (e.g., video content that depicts an instructor performing one or more actions and/or activities to be emulated and/or performed by a user of the computer system)); and displaying (908), in a second region of the workout selection user interface different from the first region, a second workout platter (e.g., user interface object and/or affordance) different from the first workout platter and associated with a second respective workout (e.g., 670b-670d) (e.g., in some embodiments, a second workout platter that is selectable to initiate a second respective workout and/or a initiate a workout session corresponding to the second respective workout; in some embodiments, the second workout platter is selectable to cause display of audio-visual content corresponding to the second respective workout (e.g., video content that depicts an instructor performing one or more actions and/or activities to be emulated and/or performed by a user of the computer system)) different from the first respective workout, wherein: in accordance with a determination that a first set of criteria is satisfied (910), including a first criterion that is satisfied when a user-defined workout plan is active for a user of the computer system (e.g., for a current user of the computer system and/or for a currently logged in user account) (e.g., when the one or more user inputs are received) (e.g., a user-defined workout plan that is associated with one or more user-selected workout days (e.g., one or more days of the week (e.g., Monday, Tuesday, Wednesday, Thursday, Friday, Saturday, and/or Sunday) and/or one or more days in a calendar month (e.g., the first of the month, the second of the month, the third of the month, and so forth)), and/or one or more user-selected workout types (e.g., one or more workout modalities (e.g., running, cycling, swimming, HIIT, yoga, core, rowing, strength, and/or dance), one or more muscle groups and/or body parts (e.g., legs, arms, shoulders, chest, and/or back), indoor workout, outdoor workout, and/or track workout)) wherein the user-defined workout plan is generated based on one or more user inputs received at a first external device (e.g., 600) (e.g., a separate computer system, a smart phone, a smart watch, a tablet, a laptop, a desktop, a wearable device, and/or head-mounted device) (e.g., an external device that is associated with and/or corresponds to the computer system; an external device that is associated with and/or corresponds to the same user and/or user account as the computer system) separate from the computer system, the first workout platter corresponds to a first workout that is selected based on the user-defined workout plan (e.g., 670c) (e.g., a first workout that is selected based on one or more user-selected workout days, a first workout that is selected based on one or more user-selected workout durations, and/or a first workout that is selected based on one or more user-selected workout types); and in accordance with a determination that a user-defined workout plan is not active (912) (e.g., when the one or more user inputs are received) (e.g., the first set of criteria is not satisfied and/or the first criterion is not satisfied), the first workout platter corresponds to a second workout different from the first workout (e.g., 670a). Displaying different workouts in a workout selection user interface based on whether a user-defined workout plan is active enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while the user-defined workout plan is active for the user of the computer system, the computer system displays, via the one or more display generation components, the workout selection user interface (e.g., 670) (e.g., a second instance of the workout selection user interface), wherein: in accordance with a determination that a first scheduled workout is scheduled for a current day according to the user-defined workout plan, the first workout platter corresponds to the first scheduled workout, and the workout selection user interface includes a first selectable option that is selectable to initiate a workout session corresponding to the first scheduled workout (e.g., FIG. 6Z); and in accordance with a determination that no workout is scheduled for the current day according to the user-defined workout plan, the first workout platter corresponds to a first future workout that is scheduled for a future date according to the user-defined workout plan, and the workout selection user interface does not include a selectable option that is selectable to initiate the first future workout (e.g., FIG. 6BB, in some embodiments, platter 670f is displayed without option 670f-1). In some embodiments, initiating a workout session corresponding to the first scheduled workout comprises initiating recording of one or more physical activity metrics (e.g., heartrate, workout time elapsed, distance traversed, and/or calories burned) for the workout session (e.g., via one or more sensors in communication with the computer system and/or one or more sensors in a first external device that is in communication with the computer system)) (in some embodiments, initiating the workout session includes recording one or more physical activity metrics at a greater frequency than prior to initiation of the workout session). In some embodiments, initiating the workout session corresponding to the first scheduled workout comprises displaying video content corresponding to the first scheduled workout (e.g., video content that depicts an instructor performing one or more actions and/or activities to be emulated and/or performed by a user of the computer system). Displaying different content in a workout selection user interface based on whether a workout is scheduled for the current day according to a user-defined workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while the user-defined workout plan is active for the user of the computer system, the computer system displays, via the one or more display generation components, the workout selection user interface (e.g., 670) (e.g., a second instance of the workout selection user interface), wherein the first platter includes a view plan option (e.g., 670f-2) that is selectable to display a plan user interface corresponding to the user-defined workout plan (e.g., a user interface that displays information about the user-defined workout plan and/or displays scheduled workouts in the user-defined workout plan). Displaying different content in a workout selection user interface based on whether a workout is scheduled for the current day according to a user-defined workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while the user-defined workout plan is active for the user of the computer system, the computer system displays, via the one or more display generation components, the workout selection user interface (e.g., 670) (e.g., a second instance of the workout selection user interface), wherein: the first workout platter corresponds to a first scheduled workout that is scheduled according to the user-defined workout plan (e.g., 670f); and the second workout platter (e.g., 670b-670d) corresponds to a first non-scheduled workout that is not scheduled according to the user-defined workout plan. Displaying various workout options, including workout options that are in the user-defined workout plans and those that aren't, enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, displaying the workout selection user interface (e.g., 670) further comprises: displaying, in a third region of the workout selection user interface, a third workout platter (e.g., 670b-670d) that corresponds to a third respective workout, wherein: in accordance with a determination that the third respective workout is scheduled for a future date according to the user-defined workout plan, the third workout platter includes a first indication that the third respective workout is scheduled for a future date according to the user-defined workout plan. Displaying an indication that a workout is scheduled for a future date enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, displaying the third workout platter that corresponds to the third respective workout further includes: in accordance with a determination that the third respective workout is not scheduled in the user-defined workout plan, forgoing display of the first indication. Forgoing display of the first indication when a workout is not scheduled for a future date enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while the user-defined workout plan is active for the user of the computer system, the computer system displays, via the one or more display generation components, the workout selection user interface (e.g., 670) (e.g., a second instance of the workout selection user interface), wherein: the first platter corresponds to a first scheduled workout according to the user-defined workout plan; and the first platter includes: a start workout option (e.g., 670e-1) that is selectable to initiate a workout session corresponding to the first scheduled workout; and a view plan option (e.g., 670e-2) that is selectable to view a user interface that corresponds to the user-defined workout plan (e.g., a user interface that displays information about the user-defined workout plan and/or displays scheduled workouts in the user-defined workout plan). In some embodiments, initiating a workout session corresponding to the first scheduled workout comprises initiating recording of one or more physical activity metrics (e.g., heartrate, workout time elapsed, distance traversed, and/or calories burned) for the workout session (e.g., via one or more sensors in communication with the computer system and/or one or more sensors in a first external device that is in communication with the computer system)) (in some embodiments, initiating the workout session includes recording one or more physical activity metrics at a greater frequency than prior to initiation of the workout session). In some embodiments, initiating the workout session corresponding to the first scheduled workout comprises displaying video content corresponding to the first scheduled workout (e.g., video content that depicts an instructor performing one or more actions and/or activities to be emulated and/or performed by a user of the computer system). Displaying an option to start a workout and an option to view workout plan information enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while displaying the workout selection user interface (e.g., 670) including the start workout option (e.g., 670e-1) and the view plan option (e.g., 670e-2), the computer system receives, via the one or more input devices, one or more user inputs corresponding to selection of the view plan option; and in response to receiving the one or more user inputs corresponding to selection of the view plan option, displays, via the one or more display generation components, a plan user interface (e.g., 678) that corresponds to the user-defined workout plan, and that displays a representation of a first scheduled workout scheduled for the current day according to the user-defined workout plan. Displaying an option to start a workout and an option to view workout plan information enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while displaying the plan user interface (e.g., 678) including the representation of the first scheduled workout (e.g., 678a-678b), the computer system receives, via the one or more input devices, one or more user inputs corresponding to selection of the representation of the first scheduled workout (e.g., a long press and/or a press and hold on the representation of the first scheduled workout (e.g., in some embodiments, via a touch-sensitive surface; and/or, in some embodiments, via a remote control)); and in response to receiving the one or more user inputs corresponding to selection of the representation of the first scheduled workout, displays a swap option (e.g., 682a) that is selectable to initiate a process for switching the first scheduled workout with a different workout (e.g., replacing the first scheduled workout with a different workout (e.g., for the user-defined workout plan)). Displaying an option to swap a workout in a workout plan enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while displaying the plan user interface (e.g., 678) including the representation of the first scheduled workout, the computer system receives, via the one or more input devices, one or more user inputs corresponding to selection of the representation of the first scheduled workout (e.g., a long press and/or a press and hold on the representation of the first scheduled workout (e.g., in some embodiments, via a touch-sensitive surface; and/or, in some embodiments, via a remote control)); and in response to receiving the one or more user inputs corresponding to selection of the representation of the first scheduled workout, displays an add option (e.g., 682d) that is selectable to add the first scheduled workout to an ordered set of workouts (e.g., a set of workouts arranged in an order (e.g., an order of playback and/or an order of performance)). Displaying an option to add a workout to a collection of workouts enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while displaying the plan user interface (e.g., 678) including the representation of the first scheduled workout, the computer system receives, via the one or more input devices, one or more user inputs corresponding to a user request to navigate (e.g., scroll) the plan user interface; and in response to receiving the one or more user inputs corresponding to the user request to navigate the plan user interface, displays, within the plan user interface, representations of one or more future scheduled workouts that are scheduled for future dates according to the user-defined workout plan (e.g., FIG. 6GG). Allowing a user to display future scheduled workouts by navigating the plan user interface enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, while displaying the plan user interface (e.g., 678) including the representation of the first scheduled workout, the computer system receives, via the one or more input devices, one or more user inputs corresponding to a user request to navigate (e.g., scroll) the plan user interface; and in response to receiving the one or more user inputs corresponding to the user request to navigate the plan user interface, displays, within the plan user interface, representations of one or more past workouts that were scheduled on past date according to the user-defined workout plan (e.g., FIG. 6FF). Allowing a user to display past plan workouts by navigating the plan user interface enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while the user-defined workout plan is active for the user of the computer system, the computer system determines that the user has completed a first plan workout according to the user-defined workout plan (e.g., a first plan workout that was scheduled in the user-defined workout plan) on the computer system (e.g., the first plan workout was displayed and/or video content corresponding to the first plan workout was displayed by the computer system and/or via the one or more display generation components (e.g., video content that includes an instruction and/or a trainer demonstrating one or more workout activities of the first plan workout)); and in response to determining that the user has completed the first plan workout on the computer system, causes the first external device (e.g., 600) to display an indication that the user completed the first plan workout (e.g., an indication that is displayed in a plan summary user interface and/or a plan user interface on the first external device). Causing a first external device to display an indication that the user completed a first plan workout when the first plan workout is completed on the computer system enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while the user-defined workout plan is active for the user of the computer system, the computer system displays, via the one or more display generation components, an indication that the user has completed a first scheduled plan workout according to the user-defined workout plan, wherein the first scheduled plan workout was completed by the user on the first external device (e.g., 600) (e.g., the first scheduled plan workout was displayed and/or video content corresponding to the first schedule plan workout was displayed by the first external device (e.g., video content that includes an instruction and/or a trainer demonstrating one or more workout activities of the first plan workout)). Displaying an indication that the user completed a workout when the user completes the workout on an external device enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the computer system (e.g., 668) and the first external device (e.g., 600) are associated with the user of the computer system (e.g., in some embodiments, are logged into the same user account corresponding to the user of the computer system). Displaying different workouts in a workout selection user interface based on whether a user-defined workout plan generated on an external device is active enhances the operability of the system and makes the user-system interface more efficient (e.g., by helping the user to provide proper inputs and reducing errors) which, additionally, reduces power usage and improves battery life of the device by enabling the user to use the system more quickly and efficiently.

Note that details of the processes described above with respect to method 900 (e.g., FIG. 9) are also applicable in an analogous manner to the methods described above and/or below. For example, method 900 optionally includes one or more of the characteristics of the various methods described above with reference to method 700 and/or 800. For example, in some embodiments, the workout plan recited in method 900 is the workout plan recited in method 700 and/or method 800. For brevity, these details are not repeated below.

FIGS. 10A-10V illustrate exemplary user interfaces for providing and accessing workout content, in accordance with some embodiments. The user interfaces in these figures are used to illustrate the processes described below, including the processes in FIG. 11.

FIG. 10A illustrates electronic device 600, which is a smart phone with touch-sensitive display 602. FIG. 10A also illustrates electronic device 688, which is an entertainment system with television 670. In the depicted embodiments, similar user interfaces and user inputs are shown on electronic device 600 and electronic device 688 to demonstrate similar features that are accessible on two different types of devices.

At FIG. 10A, electronic device 600 displays workout selection user interface 604, which was discussed above. Workout selection user interface 604 includes workout representations 1002-1002c corresponding to different workouts, and also includes collection indication 608e, which is representative of a collection of workouts, as will be discussed in greater detail below. Similarly, electronic device 668 displays workout selection user interface 670, which includes workout representations 1004a-1004c, and collection indication 1005, which corresponds to and/or is representative of a collection of workouts. At FIG. 10A, electronic device 600 detects user input 1006 (e.g., a tap input, a press input, and/or a tap and hold touch input), and electronic device 668 detects user input 1006b (e.g., a tap remote control input and/or a tap and hold remote control input).

At FIG. 10B, in response to user input 1006a, electronic device 600 displays user interface 1008a corresponding to the selected workout. In some embodiments, user input 1006a is a first type of input (e.g., a tap input or a press input). In some embodiments, a tap input on workout representation 1006a initiates a workout session, and a press input on workout representation 1006a (e.g., an input with greater than a threshold amount of force) results in display of user interface 1008a. User interface 1008a includes option 1008a-1 to initiate a workout session, and option 1008a-3. Similarly, in response to user input 1006b, electronic device 668 displays user interface 1008b corresponding to the selected workout. In some embodiments, user input 1006b is a first type of input. In some embodiments, a second type of input on workout representation 1006b initiates a workout session, and a first type of input on workout representation 1006b results in display of user interface 1008b. User interface 1008b includes option 1008b-1 to initiate a workout session, option 1008b-2 to save the workout to a library, and option 1008b-3. At FIG. 10B, electronic device 600 detects user input 1010a, and electronic device 668 detects user input 1010b.

At FIG. 10C, in response to user input 1010a, electronic device 600 displays options 1008a-4, 1008a-5, 1008a-6. Option 1008a-4 is selectable to save the workout to a library; option 1008a-5 is selectable to save the workout to a collection of workouts; option 1008a-6 is selectable to share the workout. In response to user input 1010b, electronic device 668 displays options 1008b-4, 1008b-5, 1008b-6. Option 1008b-4 is selectable to save the workout to a library, option 1008b-5 is selectable to add the workout to a collection of workouts, and option 1008b-6 is selectable to share the workout.

FIG. 10D depicts an alternative scenario in which user inputs 1006a, 1006b from FIG. 10A were different types of inputs. At FIG. 10D, in response to user input 1006a, electronic device 600 displays options 1012a-1012d. Option 1012a is selectable to initiate a workout session; option 1012b is selectable to add the workout to a library, option 1012c is selectable to add the workout to a collection of workouts; and option 1012d is selectable to share the workout. In response to user input 1006b, electronic device 668 displays options 1014a-1014c. Option 1014a is selectable to save the workout to a library of workouts; option 1014b is selectable to add the workout to a collection of workouts; and option 1014c is selectable to view the collection of workouts. At FIG. 10D, electronic device 600 detects user input 1012c corresponding to selection of option 1012c, and electronic device 668 detects user input 1016b corresponding to selection of option 1014b.

At FIG. 10E, in response to user input 1016a, the selected workout is added to a collection of workouts, and collection indication 608e is updated to indicate that there is one workout in the collection of workouts. Similarly, in response to user input 1016b, the selected workout is added to the collection of workouts, and collection indication 1005 is updated to indicate that there is one workout in the collection of workouts. In some embodiments, the collection of workouts corresponds to the user. Furthermore, as discussed above, in some embodiments, electronic device 600 and electronic device 668 correspond to the same user. In some embodiments, the collection of workouts is accessible from multiple devices, including electronic device 600 and electronic device 668, and addition of a workout to the collection of workouts on one device is also reflected on other devices corresponding to the user. For example, in some embodiments, user input 1016a on electronic device 600 would add the workout to the collection, and collection indication 1005 on electronic device 668 would be updated to reflect that there is one workout in the collection (e.g., even without input on electronic device 668). Similarly, user input 1016b on electronic device 668 would add the workout to the collection, and collection indication 608e on electronic device 600 would e updated to reflect that there is one workout in the collection (e.g., even without input on electronic device 600).

FIG. 10F depicts a scenario in which collection indication 608e and collection indication 1005 indicate that three workouts have now been added to the collection of workouts. At FIG. 10F, electronic device 600 detects user input 1018a corresponding to selection of collection indication 606e, and electronic device 668 detects user input 1018b corresponding to selection of collection indication 1005.

At FIG. 10G, in response to user input 1018a, electronic device 600 displays user interface 1020a corresponding the collection of workouts. In response to user input 1018b, electronic device 668 displays user interface 1020b corresponding to the collection of workouts. User interface 1020a displays three workouts 1020a-1, 1020a-2, 1020a-3 that are currently in the collection of workouts. User interface 1020a also includes option 1022a that is selectable to initiate a workout session corresponding to the collection of workouts. In some embodiments, workouts in the collection of workouts are arranged in an order, and a workout session corresponding to the collection of workouts automatically plays the workouts sequentially in order so that the user can perform multiple workouts in a predefined and/or user-defined order without having to provide user inputs to select a next workout that the user would like to perform. User interface 1020a and user interface 1020b indicate that the current collection of workouts has two workouts and one meditation, and has a total duration of 60 minutes. Similar to user interface 1020a, user interface 1020b displays representations 1020b-1, 1020b-2, 1020b-3 corresponding to the three workouts in the collection of workouts, and option 1022b that is selectable to initiate a workout session corresponding to the collection of workouts. At FIG. 10G, electronic device 600 detects user input 1026a corresponding to selection of option 1024a, and electronic device 600 detects user input 1026b corresponding to selection of option 1024b.

At FIG. 10H, in response to user input 1026a, electronic device 600 displays options 1028a-1, 1028a-2, 1028a-3. Option 1028a-1 is selectable to edit the collection of workouts (e.g., add, remote, and/or reorder the workouts in the collection of workouts). Option 1028a-2 is selectable to save the collection of workouts, and its current ordered set of workouts, to a library. Option 1028a-3 is selectable to remove all workouts from the collection of workouts. Similarly, in response to user input 1026b, electronic device 668 displays options 1028b-1, 1028b-2, 1028b-3. Option 1028b-1 is selectable to edit the collection of workouts (e.g., add, remote, and/or reorder the workouts in the collection of workouts). Option 1028b-2 is selectable to save the collection of workouts, and its current ordered set of workouts, to a library. Option 1028b-3 is selectable to remove all workouts from the collection of workouts. At FIG. 10H, electronic device 600 detects user input 1030a corresponding to selection of option 1028a-2, and electronic device 668 detects user input 1030b corresponding to selection of option 1028b-2.

At FIG. 10I, in response to user input 1030a, electronic device 600 displays user interface 1032a for the user to enter a name for the collection of workouts using keyboard 1032a-3. In response to user input 1030b, electronic device 600 displays user interface 1032b for the user to enter a name for the collection of workouts using keyboard 1032b-2. At FIG. 10I, electronic device 600 detects user input 1034a corresponding to selection of option 1032a-2, and user input 1034b corresponding to selection of option 1032b-2.

At FIG. 10J, in response to user input 1034a, electronic device 600 saves the collection of workouts with the name “Brett's Fit Stack.” In response to user input 1034b, electronic device 668 saves the collection of workouts with the name “Brett's Fit Stack.” As discussed above, although both electronic devices 600, 668 are displayed performing these functions, in some embodiments, one electronic device can perform the function and the saved collection of workouts can be accessed from either device 600, 668 (e.g., based on both electronic devices 600, 668 corresponding to the same user).

At FIG. 10K, electronic device 600 displays workout selection user interface 604, and electronic device 668 displays workout selection user interface 670. At FIG. 10K, electronic device 600 detects upward scrolling user input 1036a, and electronic device 668 detects upward scrolling user input 1036. At FIG. 10L, in response to user input 1036a, electronic device 600 displays scrolling of user interface 604 to reveal options 1038a-1 through 1038a-5, and in response to user input 1036b, electronic device 668 displays scrolling of user interface 670 to reveal options 1038b-1 through 1038b-5. Options 1038a-1 and 1038b-1 are selectable to view a set of workouts that the user has saved in a library. Options 1038a-2 and 1038b-2 are selectable to view a set of meditations that the user has saved in a library. Options 1838a-3 and 1038b-3 are selectable to view workout collections that the user has saved in a library. Options 1038a-4 and 1038b-4 are selectable to view workout plans (e.g., user-defined workout plans) for the user (e.g., past plans and/or current plans). Options 1038a-5 and 1038b-5 are selectable to view downloaded workouts. At FIG. 10L, electronic device 600 detects user input 1039a corresponding to selection of option 1038a-3, and electronic device 668 detects user input 1039b corresponding to selection of option 1038b-3.

At FIG. 10M, in response to user input 1039a, electronic device 600 displays user interface 1040a, which displays three workout collections 1040a-1, 1040a-2, 1040a-3 that the user has saved. In response to user input 1039b, electronic device 668 displays user interface 1040b, which displays three workout collections 1040b-1, 1040b-2, 1040b-3 that the user has saved. At FIG. 10M, electronic device 600 detects user input 1042a corresponding to selection of workout collection 1040a-1, and electronic device 668 detects user input 1042b corresponding to selection of workout collection 1040b-1.

At FIG. 10N, in response to user input 1042a, electronic device 600 displays user interface 1044a, which displays the workouts 1044a-1, 1044a-2, 1044a-3 in selected workout collection 1040a-1. Similarly, in response to user input 1042b, electronic device 668 displays user interface 1044b, which displays the workouts 1044b-1, 1044b-2, 1044b-3 in the selected workout collection 1040b-1. At FIG. 10N, electronic device 600 detects user input 1046a corresponding to selection of option 1044a-4, and electronic device 668 detects user input 1046b corresponding to selection of option 1044b-4.

At FIG. 10O, in response to user input 1046a, electronic device 600 initiates a workout session corresponding to the selected workout collection, including displaying user interface 1048a. User interface 1048a displays video content 1048a-1 corresponding the first workout in the workout collection (e.g., HIIT with Kim), workout metrics 1048a-2 for the workout session, and daily physical activity metrics 1048a-3. In response to user input 1046b, electronic device 668 initiates a workout session corresponding to the selected workout collection, including displaying user interface 1048b. User interface 1048b displays video content 1048b-1 corresponding the first workout in the workout collection (e.g., HIIT with Kim), workout metrics 1048b-2 for the workout session, and daily physical activity metrics 1048b-3.

At FIG. 10P, the first workout in the workout collection has completed. Electronic device 600 displays user interface 1050a, which includes workout metrics 1050a-1 corresponding to the first workout in the workout collection. User interface 1050a also includes indication 1050a-2 that indicates the next workout in the workout collection (HIIT with Jason), and includes options 1050a-3, 1050a-4. Option 1050a-3 is selectable to start the next workout in the workout collection. In some embodiments, the next workout in the workout collection automatically starts after a threshold duration of time. Option 1050a-4 is selectable to end the workout session early (e.g., without completing the other workouts in the workout collection). Electronic device 668 displays user interface 1050b, which includes workout metrics 1050b-1 corresponding to the first workout in the workout collection. User interface 1050b also includes indication 1050b-2 that indicates the next workout in the workout collection (HIIT with Jason), and includes options 1050b-3, 1050b-4. Option 1050b-3 is selectable to start the next workout in the workout collection. In some embodiments, the next workout in the workout collection automatically starts after a threshold duration of time. Option 1050b-4 is selectable to end the workout session early (e.g., without completing the other workouts in the workout collection).

At FIG. 10Q, the next workout in the workout collection has started, and electronic device 600 displays user interface 1052a which displays video content 1052a-1 corresponding to the next workout, workout metrics 1052a-2, and daily physical activity metrics 1052a-3. Electronic device 668 displays user interface 1052b which displays video content 1052b-1 corresponding to the next workout, workout metrics 1052b-2, and daily physical activity metrics 1052b-3.

At FIG. 10R, the second workout in the workout collection has completed. Electronic device 600 displays user interface 1054a, which includes workout metrics 1054a-1 corresponding to the second workout in the workout collection. User interface 1054a also includes indication 1054a-2 that indicates the next workout in the workout collection (HIIT with Mary), and includes options 1054a-3, 1054a-4. Option 1054a-3 is selectable to start the next workout in the workout collection. In some embodiments, the next workout in the workout collection automatically starts after a threshold duration of time. Option 1054a-4 is selectable to end the workout session early (e.g., without completing the other workouts in the workout collection). Electronic device 668 displays user interface 1054b, which includes workout metrics 1054b-1 corresponding to the second workout in the workout collection. User interface 1054b also includes indication 1054b-2 that indicates the next workout in the workout collection (HIIT with Mary), and includes options 1054b-3, 1054b-4. Option 1054b-3 is selectable to start the next workout in the workout collection. In some embodiments, the next workout in the workout collection automatically starts after a threshold duration of time. Option 1054b-4 is selectable to end the workout session early (e.g., without completing the other workouts in the workout collection).

At FIG. 10S, the final workout in the workout collection has started, and electronic device 600 displays user interface 1056a which displays video content 1056a-1 corresponding to the final workout, workout metrics 1056a-2, and daily physical activity metrics 1056a-3. Electronic device 668 displays user interface 1056b which displays video content 10526-1 corresponding to the final workout, workout metrics 1056b-2, and daily physical activity metrics 1056b-3.

At FIG. 10T, the final workout in the workout collection has completed. Electronic device 600 displays user interface 1058a, which includes workout metrics 1058a-1 corresponding to the final workout in the workout collection. User interface 1058a also includes workout collection workout metrics 1058a-2, which includes metrics aggregated over the entire workout session for the entire workout collection. User interface 1058a also includes options 1058a-3, 1058a-4. Option 1058a-3 is selectable to cease display of user interface 1058a. Option 1058a-4 is selectable to save the workout collection. Electronic device 668 displays user interface 1058b, which includes workout metrics 1058b-1 corresponding to the final workout in the workout collection. User interface 1058b also includes workout collection workout metrics 1058b-2, which includes metrics aggregated over the entire workout session for the entire workout collection. User interface 1058b also includes options 1058b-3, 1058b-4. Option 1058b-3 is selectable to cease display of user interface 1058b. Option 1058b-4 is selectable to save the workout collection.

At FIG. 10U, electronic device 600 displays user interface 1060, which includes representations 1060a-1060c of past workouts performed by the user. Representation 1060a corresponds to an outdoor walk workout the user performed on May 26, 2022. Representation 1060c corresponds to a 5-minute meditation the user previously performed. Representation 1060b corresponds to a workout collection workout the user previously performed (e.g., for a workout collection entitled “Brett's HIIT Stack”). At FIG. 10U, electronic device 600 detects user input 1062 corresponding to selection of representation 1060b.

At FIG. 10V, in response to user input 1062, electronic device 600 displays user interface 1064. User interface 1064 includes information 1064a, indicating that the user performed the workout from 7:30 am-8:30 on Wednesday May 11. User interface 1064 also includes cumulative metrics 1064b, which are workout metrics aggregated over the entire workout session for all workouts in the workout collection. User interface 1064 also includes individual workout metrics 1064c-1064c, which breaks down the workout metrics for the workout session between the three workouts in the workout collection.

FIG. 11 is a flow diagram illustrating a method for providing and accessing workout content using a computer system in accordance with some embodiments. Method 1100 is performed at a computer system (e.g., 100, 300, 500) that is in communication with one or more display generation components (e.g., a display, a touch-sensitive display, a television, a monitor, a visual output device, a 3D display, a display having at least a portion that is transparent or translucent on which images can be projected (e.g., a see-through display), a projector, a heads-up display, and/or a display controller) and one or more input devices (e.g., a touch-sensitive surface (e.g., a touch-sensitive display); a mouse; a keyboard; a remote control; a visual input device (e.g., one or more cameras (e.g., an infrared camera, a depth camera, a visible light camera, and/or a gaze tracking camera)); an audio input device; a biometric sensor (e.g., a fingerprint sensor, a face identification sensor, a gaze tracking sensor, and/or an iris identification sensor) and/or one or more mechanical input devices (e.g., a depressible input mechanism; a button; a rotatable input mechanism; a crown; and/or a dial)). Some operations in method 1100 are, optionally, combined, the orders of some operations are, optionally, changed, and some operations are, optionally, omitted.

As described below, method 1100 provides an intuitive way for providing and accessing workout content. The method reduces the cognitive burden on a user for providing and accessing workout content, thereby creating a more efficient human-machine interface. For battery-operated computing devices, enabling a user to access workout content faster and more efficiently conserves power and increases the time between battery charges.

The computer system (e.g., 600 and/or 668) receives (1102), via the one or more input devices, a first user input (e.g., 1046a and/or 1046b) (e.g., a first set of user inputs and/or one or more user inputs) (e.g., one or more touch inputs, one or more non-touch inputs, and/or one or more gestures) corresponding to a request to initiate a workout session corresponding to a first collection of workouts (in some embodiments, initiating the workout session includes initiating recording of one or more physical activity metrics (e.g., heartrate and/or calories burned) for the workout session (e.g., via one or more sensors in communication with the computer system)) (in some embodiments, initiating the workout session includes recording one or more physical activity metrics at a greater frequency than prior to initiation of the workout session), wherein the first collection of workouts includes a plurality of workouts arranged in an ordered sequence (a plurality of workouts selected by a user; and/or a plurality of workouts selected by a user and arranged in an ordered sequence determined by and/or specified by the user), including a first workout and a second workout different from the first workout.

In response to receiving the first user input (e.g., 1046a and/or 1046b), the computer system (e.g., 600 and/or 668) displays (1104), via the one or more display generation components, visual content corresponding to a first respective workout of the plurality of workouts (e.g., 1048a, 1048b, 1048a-1, and/or 1048b-1) (e.g., visual content that includes one or more visual instructions and/or demonstrations for performing a set of actions corresponding to the first respective workout).

Subsequent to displaying the visual content corresponding to the first respective workout (1106) (e.g., while displaying the visual content corresponding to the first respective workout, at the end of the first respective workout, after the first respective workout, after a threshold amount of the first respective workout has completed (e.g., 75%, 85%, or 95% of the first respective workout has completed), and/or when less than a threshold amount of the first respective workout remains (e.g., with 10 seconds, 20 seconds, 30 seconds, or 45 seconds remaining in the first respective workout)): in accordance with a determination that the first collection of workouts includes a subsequent workout that follows the first respective workout in the ordered sequence (1108) (e.g., there are remaining workouts in the first collection of workouts (e.g., the plurality of workouts), there is a subsequent and/or next workout in the ordered sequence of the plurality of workouts; and/or the first respective workout is not the last workout in the ordered sequence of workouts), the computer system displays, via the one or more display generation components, a first selectable object (e.g., 1050a-3 and/or 1050b-3) that is selectable to initiate the subsequent workout in the first collection of workouts (e.g., a next workout and/or an immediately subsequent workout in the ordered sequence of the plurality of workouts) (e.g., selectable to initiate playback and/or display of visual content corresponding to the subsequent workout in the first collection of workouts); and in accordance with a determination that the first respective workout is a final workout in the first collection of workouts (1110) (e.g., there are no remaining workouts in the first collection of workouts (e.g., the plurality of workouts) and/or the first respective workout is the last workout in the ordered sequence of workouts), the computer system displays, via the one or more display generation components, a workout summary user interface (e.g., 1058a, 1058b), including concurrently displaying: a first workout metric (e.g., average heart rate, calories burned, duration, and/or distance traversed) that corresponds to the first respective workout but does not correspond to at least some of the other workouts in the first collection of workouts (e.g., 1058a-1 and/or 1858b-1) (e.g., corresponds only to the first respective workout, and/or includes data that was recorded during the first respective workout and excludes data that was recorded during other workouts in the first collection of workouts); and a second workout metric (e.g., average heart rate, calories burned, duration, and/or distance traversed) that is cumulative for the first collection of workouts (e.g., 1058a-2 and/or 1058b-2) (e.g., corresponds to all workouts in the first collection of workouts, and/or a second workout metric that includes data recorded during all workouts in the first collection of workouts). In some embodiments, each workout in the plurality of workouts is a standalone workout that can be performed by the user. For example, in some embodiments, the computer system displays a user interface that includes a first workout platter corresponding to the first workout and a second workout platter corresponding to the second workout. In some embodiments, the first workout platter is selectable to initiate a workout session corresponding to the first workout (e.g., and does not correspond to the second workout) and the second workout platter is selectable to initiate a workout session corresponding to the second workout (e.g., and does not correspond to the first workout). In some embodiments, the first workout platter is selectable to display video content corresponding to the first workout (e.g., video content in which the user is provided with one or more visual instructions for performing a first set of actions corresponding to the first workout) without displaying video content corresponding the second workout; and the second workout platter is selectable to display video content corresponding to the second workout (e.g., video content in which the user is provided with one or more visual instructions for performing a second set of actions corresponding to the second workout) without displaying video content corresponding to the first workout. Automatically displaying different content based on whether a completed workout was the last workout in a collection enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, subsequent to displaying the visual content (e.g., 1048a, 1048b, 1048a-1, and/or 1048b-1) corresponding to the first respective workout: in accordance with a determination that the first collection of workouts includes a subsequent workout that follows the first respective workout in the ordered sequence, the computer system causes a first external device (e.g., a wearable device and/or a smartwatch) (e.g., a first external device that is associated with and/or corresponds to the computer system; a first external device that is associated with the same user as the computer system; and/or a first external device that is logged into the same user account as the computer system) to display a selectable object that is selectable to initiate the subsequent workout in the first collection of workouts (e.g., in some embodiments, in FIG. 10P, electronic device 600 and/or electronic device 668 cause another electronic device (e.g., a smart watch) to display a play option to initiate the next workout) (e.g., selectable to cause the computer system to display content (e.g., video content) corresponding to the subsequent workout in the first collection of workouts). Automatically displaying different content based on whether a completed workout was the last workout in a collection enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, subsequent to displaying the visual content (e.g., 1048a, 1048b, 1048a-1, and/or 1048b-1) corresponding to the first respective workout: in accordance with a determination that the first respective workout is a final workout in the first collection of workouts, the computer system causes the first external device to display the second workout metric that is cumulative for the first collection of workouts (e.g., in some embodiments, in FIG. 10T, electronic device 600 and/or electronic device 668 cause another electronic device (e.g., a smart watch) to display workout metrics). Automatically displaying different content based on whether a completed workout was the last workout in a collection enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, subsequent to displaying the visual content (e.g., 1048a, 1048b, 1048a-1, and/or 1048b-1) corresponding to the first respective workout: in accordance with a determination that the first collection of workouts includes a subsequent workout that follows the first respective workout in the ordered sequence, the computer system displays, concurrently with the first selectable object, an end object (e.g., 1050a-4 and/or 1050b-4) that is selectable to terminate the first collection of workouts prior to completion of the first collection of workouts (e.g., end a workout session corresponding to the first collection of workouts before completing all the workouts in the first collection of workouts). Automatically displaying different content based on whether a completed workout was the last workout in a collection enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, subsequent to displaying the visual content (e.g., 1048a, 1048b, 1048a-1, and/or 1048b-1) corresponding to the first respective workout, and subsequent to completion of a first subset of the first collection of workouts, the computer system displays the end object (e.g., 1050a-4 and/or 1050b-4); while displaying the end object, the computer system receives, via the one or more input devices, a selection input corresponding to selection of the end of object; and in response to receiving the selection input corresponding to selection of the end object: displays, via the one or more display generation components, the workout summary user interface (e.g., 1058a and/or 1058b), including concurrently displaying: the first workout metric that corresponds to the first respective workout but does not correspond to at least some of the other workouts in the first collection of workouts; and a third workout metric (e.g., average heart rate, calories burned, duration, and/or distance traversed) that is cumulative for first subset of the first collection of workouts that was completed (e.g., corresponds to all workouts in the first subset of the first collection of workouts, and/or a second workout metric that includes data recorded during all workouts in the first subset of the first collection of workouts). In some embodiments, when the first collection of workouts is terminated prematurely and/or ended before completion of all workouts in the first collection of workouts, the workout summary user interface displays workout metrics that are cumulative for the portion of the first collection of workouts that was completed prior to ending the first collection of workouts. Automatically displaying cumulative workout metrics for a completed portion of a first collection of workouts when the first collection of workouts is ended early allows for this operation to be performed without additional user input.

In some embodiments, subsequent to displaying the visual content (e.g., 1048a, 1048b, 1048a-1, and/or 1048b-1) corresponding to the first respective workout: in accordance with a determination that the first respective workout is a final workout in the first collection of workouts, the computer system displays, via the one or more display generation components (e.g., within the workout summary user interface and/or as part of the workout summary user interface), a save object (e.g., 1058a-4 and/or 1058b-4) that is selectable to save the first collection of workouts to the computer system (e.g., save the first collection of workouts within a set of collections of workouts). Automatically displaying an option to save the first collection of workouts to the computer system when the user completed the first collection of workouts enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the computer system (e.g., 600 and/or 668) displays, via the one or more display generation components, a workout selection user interface (e.g., 604 and/or 670) that includes a representation of a first workout and a representation of a second workout different from the first workout; while displaying the workout selection user interface, receiving, via the one or more input devices, one or more inputs corresponding to selection of the representation of the first workout (e.g., a long tap and/or a press and hold on the representation of the first workout); and in response to receiving the one or more inputs corresponding to selection of the representation of the first workout, displaying a first option (e.g., 1012c and/or 1014b) that is selectable to add the first workout to the first collection of workouts. In some embodiments, a different type of input on the representation of the first workout (e.g., a tap input and/or a short tap input) causes the computer system to initiate a workout session corresponding to the first workout. In some embodiments, initiating the workout session corresponding to the first workout comprises initiating recording of one or more physical activity metrics (e.g., heartrate, workout time elapsed, distance traversed, and/or calories burned) for the workout session (e.g., via one or more sensors in communication with the computer system and/or one or more sensors in a first external device that is in communication with the computer system)) (in some embodiments, initiating the workout session includes recording (e.g., at the computer system and/or at an external device) one or more physical activity metrics at a greater frequency than prior to initiation of the workout session). In some embodiments, initiating the workout session corresponding to the first workout comprises displaying video content corresponding to the first workout (e.g., video content that depicts an instructor performing one or more actions and/or activities to be emulated and/or performed by a user of the computer system). Displaying an option that is selectable to add a workout to a collection of workouts enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while displaying the first option (e.g., 1012c and/or 1014b), the computer system receives, via the one or more input devices, a selection input corresponding to selection of the first option; and in response to receiving the selection input corresponding to selection of the first option: adding the first workout to the first collection of workouts; and updating display of a collection indicator (e.g., 608e and/or 1005) that indicates how many workouts are in the first collection of workouts (in some embodiments, updating display of the collection indicator comprises displaying the collection indicator; in some embodiments, updating display of the collection indicator comprises causing the collection indicator to indicate that a workout has been added to the first collection of workouts). Updating the collection indicator to indicate that a new workout has been added to the first collection of workouts enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, updating display of the collection indicator (e.g., 608e and/or 1005) comprises updating display of the collection indicator to indicate an updated number of workouts in the first collection of workouts (e.g., causing a number displayed in the collection indicator to increase by 1 based on the first workout being added to the first collection of workouts). Updating the collection indicator to indicate that a new workout has been added to the first collection of workouts enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, the computer system displays, within the workout selection user interface (e.g., 604), the collection indicator (e.g., 608e and/or 1005); while displaying the workout selection user interface including the collection indicator, receiving, via the one or more input devices, a navigation input corresponding to a user request to navigate (e.g., scroll) the workout selection user interface; and in response to receiving the navigation input: displaying scrolling of the workout selection user interface, including: in accordance with a determination that the first collection of workouts does not have any workouts (e.g., there are no workouts in the first collection of workouts), ceasing display of the collection indicator; and in accordance with a determination that the first collection of workouts includes one or more workouts, maintaining display of the collection indicator (e.g., 608e and/or 1005) while displaying scrolling of the workout selection user interface (e.g., 604 and/or 670) (e.g., FIG. 10L). Displaying the collection indicator differently based on whether or not there are workouts in the first collection of workouts enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the computer system displays, within the workout selection user interface (e.g., 604 and/or 670), the collection indicator (e.g., 608e and/or 1005); while displaying the workout selection user interface including the collection indicator, the computer system receives, via the one or more input devices, a selection input (e.g., 1018a and/or 1018b) corresponding to selection of the collection indicator; and in response to receiving the selection input corresponding to selection of the collection indicator, displays, via the one or more display generation components, a collection page user interface (e.g., 1020a and/or 1020b) that includes representations of one or more workouts (e.g., 1020a-1 through 1020a-3 and/or 1020b-1 through 1020b-3) that are in the first collection of workouts (e.g., representations of all workouts that are in the first collection of workouts). Providing the user with a selectable option to access the collection page user interface enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the collection page user interface (e.g., 1020a and/or 1020b) comprises a start workout option (e.g., 1022a and/or 1022b) that is selectable to initiate a workout session corresponding to the first collection of workouts. In some embodiments, initiating the workout session corresponding to the first collection of workouts comprises initiating recording of one or more physical activity metrics (e.g., heartrate, workout time elapsed, distance traversed, and/or calories burned) for the workout session (e.g., via one or more sensors in communication with the computer system and/or one or more sensors in a first external device that is in communication with the computer system)) (in some embodiments, initiating the workout session includes recording (e.g., at the computer system and/or at an external device) one or more physical activity metrics at a greater frequency than prior to initiation of the workout session). In some embodiments, initiating the workout session corresponding to the first collection of workouts comprises displaying video content corresponding to the first collection of workouts (e.g., video content corresponding to a first workout in the ordered sequence of the first collection of workouts) (e.g., video content that depicts an instructor performing one or more actions and/or activities to be emulated and/or performed by a user of the computer system). Providing the user with a selectable option to start the first collection of workouts enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the collection page user interface (e.g., 1020a and/or 1020b) comprises: a first numerical indication of the number of workouts in the first collection of workouts; and a duration indicator that identifies the total duration (e.g., in time (e.g., in hours, minutes, and/or seconds)) of all workouts in the first collection of workouts (e.g., “2 WORKOUTS 1 MEDITATION” and “60 MIN” in FIG. 10G). Displaying an indication of the number of workouts in the first collection of workouts and a total duration of the first collection of workouts enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

In some embodiments, the computer system (e.g., 600 and/or 668) displays, via the one or more display generation components, a workout history user interface (e.g., 1060) that includes representations of a plurality of past workouts completed (e.g., performed and/or done) by the user for the computer system, including: a first workout representation (e.g., 1060a) that is representative of a first instance in which the user performed a first workout (e.g., a single workout; and/or not a collection of workouts); and a second workout representation (e.g., 1060b) that is representative of a second instance in which the user performed (e.g., completed and/or partially completed) the first collection of workouts. Displaying representations of past workouts, including a representation of a past collection of workouts performed by the user, enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, while displaying the workout history user interface (e.g., 1060) including the first workout representation and the second workout representation, the computer system receives, via the one or more input devices, a selection input (e.g., 1062) corresponding to selection of the second workout representation; and in response to receiving the selection input corresponding to selection of the second workout representation, displays, via the one or more display generation components, a second workout summary user interface (e.g., 1064) that corresponds to the second instance in which the user performed the first collection of workouts, including: a first set of metrics (e.g., 1064c-1064c) that correspond to a first performed workout that the user completed during the second instance in which the user performed the first collection of workouts (in some embodiments, the first set of metrics does not correspond to any other workouts the user performed and/or completed during the second instance in which the user performed the first collection of workouts (e.g., the first set of metrics includes workout metrics that were recorded during the first performed workout and not during any other workouts)); and a second set of metrics (e.g., 1064c-1064c) different from the first set of metrics that correspond to a second performed workout that is different from the first performed workout and that the user completed during the second instance in which the user performed the first collection of workouts (in some embodiments, the second set of metrics does not correspond to any other workouts the user performed and/or completed during the second instance in which the user performed the first collection of workouts (e.g., the second set of metrics includes workout metrics that were recorded during the second performed workout and not during any other workouts)). Allowing a user to select the second workout representation to view workout metrics for the previous instance in which the user completed the first collection of workouts enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, the second workout summary user interface (e.g., 1064) includes a first selectable option (e.g., in some embodiments, selection of 1064a causes display of 1044a) that is selectable to display a first collection page user interface that includes representations of one or more workouts that are in the first collection of workouts (e.g., representations of all workouts that are in the first collection of workouts). In some embodiments, the first collection page user interface also includes a start workout option that is selectable to initiate a workout session corresponding to the first collection of workouts. Providing the user with an option to display the first collection page user interface from the second workout summary user interface enhances the operability of the system and makes the user-system interface more efficient (e.g., by helping the user to provide proper inputs and reducing errors) which, additionally, reduces power usage and improves battery life of the device by enabling the user to use the system more quickly and efficiently.

In some embodiments, in accordance with a determination that the first collection of workouts is a saved collection (e.g., saved to the computer system; and/or saved in a set of collections of workouts) and has a first collection name, the second workout representation (e.g., 1060b) includes the first collection name; and in accordance with a determination that the first collection of workouts is not a saved collection (e.g., is not saved to the computer system, is not saved in a set of collections of workouts, and/or has not been saved to the computer system by the user), the second workout representation includes a default name different from the first collection name (e.g., a default name indicative of the first collection of workouts being an unsaved collection). Displaying the second workout representation different based on whether or not the first collection of workouts is a saved collection enhances the operability of the system and makes the user-system interface more efficient (e.g., by helping the user to provide proper inputs and reducing errors) which, additionally, reduces power usage and improves battery life of the device by enabling the user to use the system more quickly and efficiently. Doing so also provides the user with feedback about a state of the device.

Note that details of the processes described above with respect to method 1100 (e.g., FIG. 11) are also applicable in an analogous manner to the methods described above. For example, method 1100 optionally includes one or more of the characteristics of the various methods described above with reference to methods 700, 800, and/or 900. For example, in some embodiments, the computer system recited in methods 700, 800, and/or 900 is the computer system recited in method 1100. For brevity, these details are not repeated below.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to improve the delivery of workout content. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to deliver targeted workout content that is of greater interest to the user. Accordingly, use of such personal information data enables users to have calculated control of the delivered workout content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of workout content delivery services, the present technology can be configured to allow users to select to “opt in” or “opt out” of participation in the collection of personal information data during registration for services or anytime thereafter. In another example, users can select not to provide workout-associated data for targeted workout content delivery services. In yet another example, users can select to limit the length of time workout-associated data is maintained or entirely prohibit the development of a baseline workout profile. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A computer system configured to communicate with one or more display generation components and one or more input devices, comprising:
one or more processors; and
memory storing one or more programs configured to be executed by the one or more processors, the one or more programs including instructions for:
receiving, via the one or more input devices, a first set of user inputs, including:
one or more user inputs corresponding to user selection of one or more workout days; and
one or more user inputs corresponding to user selection of one or more workout types;
in response to receiving the first set of user inputs, displaying, via the one or more display generation components, a workout plan user interface, including:
a representation of a first day that is selected based on the one or more user inputs corresponding to user selection of one or more workout days;
a representation of a second day different from the first day that is selected based on the one or more user inputs corresponding to user selection of one or more workout days;
an indication of a first workout corresponding to the first day and that is selected based on the one or more user inputs corresponding to user selection of one or more workout types; and
an indication of a second workout corresponding to the second day and that is selected based on the one or more user inputs corresponding to user selection of one or more workout types.

2. The computer system of claim 1, wherein the one or more user inputs corresponding to user selection of one or more workout days specifies a number of workout days per week.

3. The computer system of claim 1, wherein the one or more user inputs corresponding to user selection of one or more workout days specifies one or more days of the week.

4. The computer system of claim 1, wherein the first set of user inputs further include:
one or more user inputs corresponding to identification of one or more workout durations.

5. The computer system of claim 4, wherein the one or more user inputs corresponding to identification of one or more workout durations comprises identification of a single workout duration value.

6. The computer system of claim 4, wherein the one or more user inputs corresponding to identification of one or more workout durations comprises identification of a plurality of workout duration values.

7. The computer system of claim 6, wherein the plurality of workout duration values includes:
a first workout duration value corresponding to a first day of the one or more workout days; and
a second workout duration value corresponding to a second day of the one or more workout days different from the first day.

8. The computer system of claim 1, wherein the first set of user inputs further comprises:
one or more user inputs corresponding to user selection of one or more trainers of a plurality of trainers.

9. The computer system of claim 8, the one or more programs further including instructions for:
displaying, via the one or more display generation components, representations of a first subset of the plurality of trainers, wherein the first subset is selected from the plurality of trainers based on the one or more user inputs corresponding to user selection of one or more workout types.

10. The computer system of claim 9, wherein:
the one or more user inputs corresponding to user selection of one or more trainers defines a first set of user-selected trainers;
the first workout corresponds to a first trainer of the first set of user-selected trainers; and
the second workout corresponds to a second trainer of the plurality of trainers that is not in the first set of user-selected trainers.

11. The computer system of claim 1, wherein the first set of user inputs further comprises:
one or more user inputs corresponding to user selection of one or more music types of a plurality of types.

12. The computer system of claim 1, wherein the first set of user inputs further comprises:
one or more user inputs defining a first workout plan duration.

13. The computer system of claim 1, wherein:
the first set of user inputs correspond to a user request to generate a workout plan;
the workout plan user interface corresponds to the workout plan; and
the one or more programs further include instructions for:
subsequent to displaying the workout plan user interface, displaying, via the one or more display generation components, a workout selection user interface that comprises a plurality of workout platters corresponding to a plurality of workouts, including:
displaying, in a first region of the workout selection user interface, a first workout platter associated with the first workout; and
displaying, in a second region of the workout selection user interface, a second workout platter associated with a third workout different from the first workout, wherein the third workout is not part of the workout plan.

14. The computer system of claim 1, the one or more programs further including instructions for:
displaying, via the one or more display generation components, representations of a plurality of workout types, wherein:
the one or more user inputs corresponding to user selection of the one or more workout types comprises one or more user inputs interacting with the representations of the plurality of workout types; and
the plurality of workout types are selected for display based on previous workouts completed by a user of the computer system.

15. The computer system of claim 1, wherein:
the first set of user inputs correspond to a user request to generate a first workout plan;
the workout plan user interface corresponds to the first workout plan; and
the one or more programs further include instructions for:
subsequent to displaying the workout plan user interface, displaying, via the one or more display generation components, a first workout selection user interface that comprises a first plurality of workout platters corresponding to a first plurality of workouts, including:
displaying, in a first display region of the first workout selection user interface, a first platter associated with the first workout, wherein the first workout platter is selected for display in the first region of the workout selection user interface based on the first workout being part of the first workout plan.

16. A non-transitory computer-readable storage medium storing one or more programs configured to be executed by one or more processors of a computer system that is in communication with one or more display generation components and one or more input devices, the one or more programs including instructions for:
receiving, via the one or more input devices, a first set of user inputs, including:
one or more user inputs corresponding to user selection of one or more workout days; and
one or more user inputs corresponding to user selection of one or more workout types;
in response to receiving the first set of user inputs, displaying, via the one or more display generation components, a workout plan user interface, including:
a representation of a first day that is selected based on the one or more user inputs corresponding to user selection of one or more workout days;
a representation of a second day different from the first day that is selected based on the one or more user inputs corresponding to user selection of one or more workout days; and
an indication of a first workout corresponding to the first day and that is selected based on the one or more user inputs corresponding to user selection of one or more workout types; and
an indication of a second workout corresponding to the second day and that is selected based on the one or more user inputs corresponding to user selection of one or more workout types.

17. A method, comprising:
at a computer system that is in communication with one or more display generation components and one or more input devices:
receiving, via the one or more input devices, a first set of user inputs, including:
one or more user inputs corresponding to user selection of one or more workout days; and
one or more user inputs corresponding to user selection of one or more workout types;
in response to receiving the first set of user inputs, displaying, via the one or more display generation components, a workout plan user interface, including:
a representation of a first day that is selected based on the one or more user inputs corresponding to user selection of one or more workout days;
a representation of a second day different from the first day that is selected based on the one or more user inputs corresponding to user selection of one or more workout days; and
an indication of a first workout corresponding to the first day and that is selected based on the one or more user inputs corresponding to user selection of one or more workout types; and
an indication of a second workout corresponding to the second day and that is selected based on the one or more user inputs corresponding to user selection of one or more workout types.
